SEQUENCE LISTING (62 sequences)

SEQ ID NO: 1 - protein, 578 aa, Homo sapiens
VNAIPDQCSPLPCNEDGYMSCKDGKASFTCTCKPGWQGEKCEFDINECKDPSNINGGCSQICDNTPGSYHCSCKNGFVMLSNKKDCKDVDECSLKPSICGTAVCKNIPGDFECECPEGYRYNLKSKSCEDIDECSENMCAQLCVNYPGGYTCYCDGKKGFKLAQDQKSCEVVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNSALAEDQVDPRLIDGK

SEQ ID NO: 2 - protein, 188 aa, Homo sapiens
VNAIPDQCSPLPCNEDGYMSCKDGKASFTCTCKPGWQGEKCEFDINECKDPSNINGGCSQICDNTPGSYHCSCKNGFVMLSNKKDCKDVDECSLKPSICGTAVCKNIPGDFECECPEGYRYNLKSKSCEDIDECSENMCAQLCVNYPGGYTCYCDGKKGFKLAQDQKSCEVVSALAEDQVDPRLIDGK

SEQ ID NO: 3 - protein, 647 aa, Macaca fascicularis
ANSMLEETKQGNLERECIEELCNKEEAREVFENDPETDYFYPKYLVCLRSFQSGLFTAARQSTDAYPDLRSCVNAIPDQCSPLPCNEDGYMSCKDGKASFTCTCKPGWQGEKCEFDINECKDPSNINGGCSQICDNTPGSYHCSCKSGFVMLSNKKDCKDVDECSLKPNMCGTAVCKNIPGDFECECPEGYRYNLKSKSCEDVDECSENMCAQLCVNYPGGYTCYCDGKKGFKLAQDQKSCEAVSVCLPLNLDTKYELLYLAEQFAGVVLYLKFRLPEISRFSAEFDFRTYDSQGVILYAESIDHSAWLLIALRGGKIEVQLKNEHTSKITTGGDIINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAEFHIDYNNGSNAEGWHINVTLNIRPSMGTGVMLALVSSNNTVPFAVSLVDSTSEKSQDIVISVENTVIYRIQALSLCSNQRSHLEFRVNRNNLELLTPLKIETISQEELQTQLAILDKAMKGKVATYLGGLPDVPFSATPVNAFYNGCMEVNINGVELDLDEAISKHNDIRAHSCPSVWKKTKNSEDQVDPRLIDGK

SEQ ID NO: 4 - protein, 107 aa, Unknown
  Mouse
QIVLTQSPALLSASPGEKVTMTCSASSSVSYMYWYQQKPGSSPKPWIYVTSNLASGVPARFSGSGSGTSYSLTISSMEAEDAATYYCHQWSSDPYTFGGGTKLEIRR

SEQ ID NO: 5 - protein, 119 aa, Unknown
  Mouse
QVQLQQPGAELVRPGTSVKLSCKASGYTVTSYWMNWIKQRPEQGLEWIGRIDPNDSETLYNQKFKDKVILTVDKSSSTVYIQLSSLTSEDSAVYYCARWAGSGYAMDYWGQGTSVTVSS

SEQ ID NO: 6 - protein, 107 aa, Unknown
  Mouse
QIALTQSPAIMSASPGEKVTMTCSVNSSVSYMHWYQQKPGSSPRLWIYDTSNLVSGVPGRFSGSGSGTSYSLTLSSMEAEDAATYYCQQYSGYLYTFGGGTKLEIKR

SEQ ID NO: 7 - protein, 119 aa, Unknown
  Mouse
EVKLEESGGGLVQPGGSMKLSCAASGFTFSDAWMDWVRQSPEKGLEWVAEVRSKADNHATYYAESVKGRFTISRDDSKSAVYLQMNSLRPEDTGIYYCTLTTAFLFDSWGQGTTLTVSS

SEQ ID NO: 8 - protein, 107 aa, Unknown
  Mouse
QIVLTQSPAIMSASPGEKVTMTCSGRSSENYMYWYQQKPGSPPRLWIYDTSNLVSGVPARFSGSRSGTSYSLTISSMEAEDAATYYCQQFNGYPYTFGGGTKLEIRR

SEQ ID NO: 9 - protein, 119 aa, Unknown
  Mouse
EVKLEESGGGLVQPGGSMKLSCAASGFTFSDAWMGWVRQSPEKGLEWVAEIRGKANNHATYYSESVKGRFTISRDDSKSSVYLQMNSLRAEDTGIFYCCLITAFLFDYWGQGTTLTVSS

SEQ ID NO: 10 - protein, 107 aa, Unknown
  Mouse
QIALTQSPAIMSASPGEKVTMTCSVSSSVSYMHWYQQKPGSSPRLWIYDTSNLVSGVPGRFSGSGSGTSYSLTLSSMEAEDAATYYCQQYSGYLYTFGGGTKLEIKR

SEQ ID NO: 11 - protein, 119 aa, Unknown
  Mouse
EVKLEESGGGLVQPGGSMKLSCAASGFTFSDAWMDWVRQSPEKGLEWVAEIRSKANNHATYYAESVKGRFTISRDDSKSGVYLQMNSLRPEDTGIYYCTLTTAFLFDYWGQGTTLTVSS

SEQ ID NO: 12 - protein, 107 aa, Unknown
  Mouse
QIVLTQSPALMSASPGERVTMTCSASSSVSYMYWYQQKPRSSPKPWIYSTSNLASGVPARFSGSGSGTSYSLTISSMEAEDAATYYCQQWSSNPYTFGGGTKLEIKR

SEQ ID NO: 13 - protein, 119 aa, Unknown
  Mouse
QVQLQQPGTELVRPGASVKLSCKASGYTFTSYWMNWVQQRPEQGLEWIGRIDPYDTETHYNQKFEDKAILTVDKSSSTAYMQLSSLTSEDSAVYYCARWAGSSYAMDYWGQGTSVTVSS

SEQ ID NO: 14 - protein, 108 aa, Unknown
  Mouse
DIVLTQSPATLSVTPGDSVSLSCRASQSISNNLHWYQQKSHESPRLLIKFVSQSISGIPSRFSGSGSGTDFTLSINSVETEDFGMYFCQQSNSWPWTFGGGTKLEIKR

SEQ ID NO: 15 - protein, 112 aa, Unknown
  Mouse
EVQLQESGPSLVKPSQTLSLTCSVTGDSITSGYWNWIRKFPGNKLEYMGYISFSGSTYYNPSLKSRISITRDTSKNQYYLQLNSVTTEDTATYYCAREFAYWGQGTLVTVSA

SEQ ID NO: 16 - protein, 109 aa, Unknown
  Mouse
ENVLTQSPAIMSASPGEKVTMTCRASSSVSSSYLHWYQQRSGASPKLWIYGTSNLASGVPARFSGSGSGTSYSLTISSVEAEDAATYYCQQYSGYPRTFGGGTKLEIKR

SEQ ID NO: 17 - protein, 123 aa, Unknown
  Mouse
EVLLQQSGPELVKPGASVKIPCKASGYTFTDYNMDWVKQSHGKSLEWIGDINPNNGDTFYNQKFKGKATLTVDKSSSTAYMELRSLTSEDTAVYYCARSEIFTTAPDYGMGYWGQGTSVTVSS

SEQ ID NO: 18 - protein, 109 aa, Unknown
  Mouse
ENVLTQSPAIMSASPGEKVTMTCRASSSVSSSYLHWYRQKSGASPKLWIYSTSNLASGVPARFSGSGSGTSYSLTISRVEAEDAATYYCQQYSGYPRTFGGGTKVEIKR

SEQ ID NO: 19 - protein, 123 aa, Unknown
  Mouse
EVLLQQSGPELVKPGASVKITCKASGYTFTDYNMDWVKQSHGKSLEWIGDIDPNNGGTIYNQEFTGKATLTVDTSSSTAYMELRSLTSEDTAVYYCVRSEIFTTAPDYGVDYWGQGTSVTVSS

SEQ ID NO: 20 - protein, 112 aa, Unknown
  Mouse
NIVLTQSPASLAVSLGQRATISCRASESVDSYGNSFMHWYQQRPGQPPRLLIYLASNLESGVPARFSGSGSRTDFTLTIDPVEADDAATYYCQQNNEDPWTFGGGTRLEIKR

SEQ ID NO: 21 - protein, 117 aa, Unknown
  Mouse
QVQLLQSGAELARPGASVKLSCKASGYTFTDYYISWVTTGQGLEWIGEINPGSGDPYYNEKFKGKATLTADKSSSTAYMHLSSLTSEDSAVYFCARSDDGKGYFDVWGAGTTVTVSS

SEQ ID NO: 22 - protein, 112 aa, Unknown
  Mouse
DIVLTQSPASLAVSLGQRATISCRASKSVSASGYSYMHWYQQKPGQPPKLLIYLASNLESGVPARFSGSGSGTDFTLNIHPVEEEDAATYYCQHSRELPLTFGAGTKLELKR

SEQ ID NO: 23 - protein, 118 aa, Unknown
  Mouse
EIQLQQSGPELVKPGASVKVSCKASGYSFTDYNMYWVKQSHGKSLEWIGYIDPYNGGTSYNQKFKGKATLTVDKSSSTAFMHLNSLTSEDSAVYYCARERDYWYFDVRGAGTTVTVSS

SEQ ID NO: 24 - protein, 108 aa, Unknown
  Mouse
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPYTFGGGTKLEIKR

SEQ ID NO: 25 - protein, 119 aa, Unknown
  Mouse
QVQLQQPGAELVRPGASVKLSCKASGYTFTSYWINWVKQRPEQGLEWIGRIDPYDSETHYNQKFKDKAILTVDKSSSTAYMQLSSLTSEDSAVYYCARWGGSGYAMDYWGQGTSATVSS

SEQ ID NO: 26 - protein, 107 aa, Unknown
  Mouse
QIVLTQSPAIMSASPGEKVTITCSATSSVTYMHWFQQKPGTSPKLWIFSTSNLASGVPPRFSGSGSGTSYSLTISRMEAEDAATYYCQQRSSYPPTFGGGTKLEIKR

SEQ ID NO: 27 - protein, 115 aa, Unknown
  Mouse
QVHLKESGPGLVAPSQNLSITCTVSGFSLTGYGVSWVRQPPGKGLEWLGMIWGDGTTDYNSTLKSRLSISKDNSKSQVFLKMNSLQIDDTARYYCARDPGAMDYWGQGTSVTVSS

SEQ ID NO: 28 - protein, 112 aa, Unknown; identical to SEQ ID NO: 22
  Mouse

SEQ ID NO: 29 - protein, 118 aa, Unknown; identical to SEQ ID NO: 23
  Mouse

SEQ ID NO: 30 - protein, 113 aa, Unknown
  Mouse
DVVMTQTPLTLSVTIGQPASISCKSSQSLLDSDGKTYLNWMLQRPGQSPKRLIYLVSKLDSGVPDRFTGSGSGTDFTLKISRVEAEDLGVYYCWQGTHFPRTFGGGTKLEIRR

SEQ ID NO: 31 - protein, 114 aa, Unknown
  Mouse
EVQLQQSGAELVRSGASVKLSCTASGFNIKDNYIHWVKQRPEQGLEWIGWIDPENGDTEYAPKFQGKATMTADTSSNTAYLQLSSLTSEDTAVYYCSAYGAAYWGQGTLVTVSA

SEQ ID NO: 32 - protein, 108 aa, Unknown
  Mouse
DIVMTQSHKFMSTSVGDRVSITCKASQDVSTGVAWYQQKPGQSPKLLIFSASSRYSGVPDRFTGSGSGTDFTFTISSVQAEDLAVYYCQQHYSSPRTFGGGTKLEIKR

SEQ ID NO: 33 - protein, 119 aa, Unknown
  Mouse
QIQLVQSGPELKKPGETVKISCKASGYTFSDYSMHWVKQTPGEGLKWMGWMNTGTGEPTYADDFKGRFAFSLETSATTAHLQINNLKNEDTATYFCARRLYYGTGLAYWGQGTLVTVSA

SEQ ID NO: 34 - protein, 112 aa, Unknown
  Mouse
NIVLTQSPASLAVSLGQRATISCRASESVDSYGNSFMHWYQQRPGQPPKLLIYLASNLESGVPARFSGSGSRTDFTLTIDPVEADDAATYYCQQNNEDPWTFGGGTKLEIKR

SEQ ID NO: 35 - protein, 117 aa, Unknown
  Mouse
QVQLLQSGAELARPGASVKLSCKASGYTFTDYYISWVTTGQGLEWIGEINPGSDIPYYNEKFKGKATLTADKSSSTAYMHFSSLTSEDSAVYFCARSDDGKGYFDVWGAGTTVTVSS

SEQ ID NO: 36 - protein, 107 aa, Unknown
  Mouse
QIVLTQSPAIMSASPGEKVTMTCSASSSVSYMYWYQQKPGSSPRILIYDTSNLASGVPVRFSGSGSGTSYSLTISRMEAEDAATYYCQQWSSYPLTFGAGTKLELKR

SEQ ID NO: 37 - protein, 117 aa, Unknown
  Mouse
QVTLKESGPGILQPSQTLSLTCSFSGFSLSTSGMGVSWIRQPSGKGLEWLAHIYWDDDKRYNPSLKSRLTISRDTSSNQVFLKITSVDTADTATYYCALYGNYGDYWGQGTTLTVSS

SEQ ID NO: 38 - protein, 108 aa, Unknown
  Mouse
DIQMSQSPSSLSASLGDTVTITCHASQNINIWLSWYQQKPGNILTLLIYKASNLYTGVPSRFSGSGSGTDFTLTISSLQPEDIATYYCQQGQSFPLTFGTGTRLELKR

SEQ ID NO: 39 - protein, 120 aa, Unknown
  Mouse
QVQLQQSGAEMVRPGSSVKISCKASGYAFSSHWMNWVKQRPGQGLEWIGQIYPGDGDTNYNGKFRGKAIVTADTSSSTAYLQLSSLTSEDTAVFFCAPGYHGTSYWFAYWGQGTLVTVSA

SEQ ID NO: 40 - protein, 107 aa, Unknown
  Mouse
QIVLSQSPAILSASPGEKVTMTCRASSSVSYMYWYQQKPGSSPKPWIYATSNLASGVPARFSGSGSGTSYSLTISRVEAEDAATYYCQQWSSIPPTFGGGTKLEIKR

SEQ ID NO: 41 - protein, 119 aa, Unknown
  Mouse
QVQLQQPGAELVRPGASVKLSCKASGYTFTSYWINWVKQRPEQGLEWIGRIDPYDSETHYNQKFKDKAILTVDKSSSTAYMQLSSLTSEDSAVYYCARWGGSGYAMDYWGQGTSVTVSS

SEQ ID NO: 42 - protein, 107 aa, Unknown
  Mouse
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELKR

SEQ ID NO: 43 - protein, 119 aa, Unknown
  Mouse
QVTLKESGPGILQPSQTLSLTCSFSGFSLNTSGMGVSWIRQPSGKGLEWLAHIYWDDDKRYNPSLKSRLTISKDTSRNQVFLKITSVDTEDTATYYCAKYGIPYAVDYWGQGTSVTVSS

SEQ ID NO: 44 - protein, 107 aa, Unknown
  Mouse
ETTVTQSPASLSMAIGEKVTIRCITSTDIDDDVNWYQQKPGEPPKLLISEGNILRPGVPSRFSSSGYGTDFVFSIENMLSEDVGDYYCLQSDTLPTFGGGTKLEIKR

SEQ ID NO: 45 - protein, 119 aa, Unknown
  Mouse
QVQLKESGPGLVAPSQSLSITCTVSGFSLTSYGVHWVRQPPGKGLGWLGVIWAGGRTDYNSALMSRLSISKDNSKSQVFLKMNSLQTDDTAIYYCARDYGNYNYAMDYWGQGISVTVSS

SEQ ID NO: 46 - protein, 676 aa, Homo sapiens
MRVLGGRCGALLACLLLVLPVSEANFLSKQQASQVLVRKRRANSLLEETKQGNLERECIEELCNKEEAREVFENDPETDYFYPKYLVCLRSFQTGLFTAARQSTNAYPDLRSCVNAIPDQCSPLPCNEDGYMSCKDGKASFTCTCKPGWQGEKCEFDINECKDPSNINGGCSQICDNTPGSYHCSCKNGFVMLSNKKDCKDVDECSLKPSICGTAVCKNIPGDFECECPEGYRYNLKSKSCEDIDECSENMCAQLCVNYPGGYTCYCDGKKGFKLAQDQKSCEVVSVCLPLNLDTKYELLYLAEQFAGVVLYLKFRLPEISRFSAEFDFRTYDSEGVILYAESIDHSAWLLIALRGGKIEVQLKNEHTSKITTGGDVINNGLWNMVSVEELEHSISIKIAKEAVMDINKPGPLFKPENGLLETKVYFAGFPRKVESELIKPINPRLDGCIRSWNLMKQGASGIKEIIQEKQNKHCLVTVEKGSYYPGSGIAQFHIDYNNVSSAEGWHVNVTLNIRPSTGTGVMLALVSGNNTVPFAVSLVDSTSEKSQDILLSVENTVIYRIQALSLCSDQQSHLEFRVNRNNLELSTPLKIETISHEDLQRQLAVLDKAMKAKVATYLGGLPDVPFSATPVNAFYNGCMEVNINGVQLDLDEAISKHNDIRAHSCPSVWKKTKNS

SEQ ID NO: 47 - DNA, 20 bp, Artificial Sequence
  Synthetic Primer
cccttgaccaggcatcccag

SEQ ID NO: 48 - DNA, 36 bp, Artificial Sequence
  Synthetic Primer
gctctagactaacactcattcctgttgaagctcttg

SEQ ID NO: 49 - protein, 106 aa, Artificial Sequence
  light chain variable domain (VL) of humanized monoclonal antibodies
EIVLTQSPATLSLSPGERATLSCRASSSVSYMYWYQQKPGQAPRLLIYATSNLASGIPARFSGSGSGTDFTLTISSLEPEDFAVYYCQQWSSIPPTFGGGTKVEIK

SEQ ID NO: 50 - protein, 119 aa, Artificial Sequence
  heavy chain variable domain (VH) of humanized monoclonal antibodies
QVQLVQSGAEVKKPGASVKVSCKASGYTFTSYWINWVRQAPGQGLEWMGRIDPYDSETHYAQKFQGRVTMTRDTSTSTVYMELSSLRSEDTAVYYCARWGGSGYAMDYWGQGTTVTVSS

SEQ ID NO: 51 - protein, 106 aa, Artificial Sequence
  light chain variable domain (VL) of humanized monoclonal antibodies
EIVLTQSPATLSLSPGERATLSCRASSSVSYMYWYQQKPGQAPRPLIYATSNLASGIPARFSGSGSGTDFTLTISSLEPEDFAVYYCQQWSSIPPTFGGGTKVEIK

SEQ ID NO: 52 - protein, 119 aa, Artificial Sequence
  heavy chain variable domain (VH) of humanized monoclonal antibodies
QVQLVQSGAEVKKPGASVKVSCKASGYTFTSYWINWVRQAPGQGLEWMGRIDPYDSETHYAQKFQGRVTLTRDTSTSTAYMELSSLRSEDTAVYYCARWGGSGYAMDYWGQGTTVTVSS

SEQ ID NO: 53 - protein, 106 aa, Artificial Sequence
  light chain variable domain (VL) of humanized monoclonal antibodies
EIVLTQSPATLSLSPGERATLSCRASSSVSYMYWYQQKPGQAPRPWIYATSNLASGIPARFSGSGSGTDFTLTISSLEPEDFAVYYCQQWSSIPPTFGGGTKVEIK

SEQ ID NO: 54 - protein, 119 aa, Artificial Sequence
  heavy chain variable domain (VH) of humanized monoclonal antibodies
QVQLVQSGAEVKKPGASVKVSCKASGYTFTSYWINWVRQAPGQGLEWMGRIDPYDSETHYAQKFQGRVTLTVDTSTSTAYMELSSLRSEDTAVYYCARWGGSGYAMDYWGQGTTVTVSS

SEQ ID NO: 55 - protein, 119 aa, Artificial Sequence
  heavy chain variable domain (VH) of humanized monoclonal antibodies
QVQLVQSGAEVKKPGASVKVSCKASGYTFTSYWINWVRQAPGQGLEWMGRIDPYDSETHYAQKFQGRVTLTRDKSTSTAYMELSSLRSEDTAVYYCARWGGSGYAMDYWGQGTTVTVSS

SEQ ID NO: 56 - protein, 213 aa, Artificial Sequence
  light chain (LC) of humanized monoclonal antibodies
EIVLTQSPATLSLSPGERATLSCRASSSVSYMYWYQQKPGQAPRLLIYATSNLASGIPARFSGSGSGTDFTLTISSLEPEDFAVYYCQQWSSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 446 aa, Artificial Sequence
  heavy chain (HC) of humanized monoclonal antibodies
QVQLVQSGAEVKKPGASVKVSCKASGYTFTSYWINWVRQAPGQGLEWMGRIDPYDSETHYAQKFQGRVTMTRDTSTSTVYMELSSLRSEDTAVYYCARWGGSGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 58 - protein, 213 aa, Artificial Sequence
  light chain (LC) of humanized monoclonal antibodies
EIVLTQSPATLSLSPGERATLSCRASSSVSYMYWYQQKPGQAPRPLIYATSNLASGIPARFSGSGSGTDFTLTISSLEPEDFAVYYCQQWSSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 446 aa, Artificial Sequence
  heavy chain (HC) of humanized monoclonal antibodies
QVQLVQSGAEVKKPGASVKVSCKASGYTFTSYWINWVRQAPGQGLEWMGRIDPYDSETHYAQKFQGRVTLTRDTSTSTAYMELSSLRSEDTAVYYCARWGGSGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 213 aa, Artificial Sequence
  light chain (LC) of humanized monoclonal antibodies
EIVLTQSPATLSLSPGERATLSCRASSSVSYMYWYQQKPGQAPRPWIYATSNLASGIPARFSGSGSGTDFTLTISSLEPEDFAVYYCQQWSSIPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 446 aa, Artificial Sequence
  heavy chain (HC) of humanized monoclonal antibodies
QVQLVQSGAEVKKPGASVKVSCKASGYTFTSYWINWVRQAPGQGLEWMGRIDPYDSETHYAQKFQGRVTLTVDTSTSTAYMELSSLRSEDTAVYYCARWGGSGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 446 aa, Artificial Sequence
  heavy chain (HC) of humanized monoclonal antibodies
QVQLVQSGAEVKKPGASVKVSCKASGYTFTSYWINWVRQAPGQGLEWMGRIDPYDSETHYAQKFQGRVTLTRDKSTSTAYMELSSLRSEDTAVYYCARWGGSGYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2016/0297892 A1
(43) Pub. Date: Oct. 13, 2016

(54) Novel Methods and Antibodies for Treating Coagulapathy

(71) Applicant: NOVO NORDISK A/S, Bagsvaerd (DK)

(72) Inventors: Helle Heibroch Petersen, Koebenhavn V (DK); Mette Brunsgaard Hermit, Jyllinge (DK); Heidi Lindgreen Holmberg, Oelsted (DK); Berit Olsen Krogh, Roedovre (DK); Kristian Kjaergaard, Ballerup (DK); Mette Dahl Andersen, Vaerloese (DK); Rune Salbo, Hvidovre (DK); Emily Waters, Frederiksberg (DK); Lisbeth Moreau Andersen, Broenshoej (DK); Kristoffer Winther Balling, Roedovre (DK)

(21) Appl. No.: 15/033,835

(22) PCT Filed: Nov. 7, 2014
(86) PCT No.: PCT/EP2014/074043

§ 371 Date: May 2, 2016

Related U.S. Application Data
(60) Provisional application No. 61/901,496, filed on Nov. 8, 2013.

(30) Foreign Application Priority Data
Nov. 7, 2013 (EP) 13191940.9
Jul. 4, 2014 (EP) 14175858.1

(51) Int. Cl. C07K 16/36

(52) U.S. Cl. CPC C07K 16/36; C07K 2317/76; C07K 2317/34; A61K 2039/505

(57) ABSTRACT

The present invention relates to pro-coagulant human Protein S inhibitors, such as antibodies or antigen-binding fragments thereof that can be administered subcutaneously as prophylactic treatment for haemophilia patients regardless of inhibitor status and without interfering with non-coagulant functions of Protein S.

### INCORPORATION BY REFERENCE OF THE SEQUENCE LISTING

The Sequence Listing, entitled “SEQUENCE LISTING” was created on 6 Nov. 2014 and is incorporated herein by reference.

### FIELD OF THE INVENTION

The present invention relates to inhibitors such as antibodies that specifically bind to Protein S.

### BACKGROUND

In subjects with a coagulopathy, such as in human beings with haemophilia A and B, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding, or damage to internal organs, such as the joints. Subjects such as human beings with haemophilia A and B may receive coagulation factor replacement therapy such as exogenous Factor VIII (FVIII) or Factor IX (FIX), respectively. However, such patients are at risk of developing “inhibitors” (antibodies) to such exogenous factors, rendering formerly efficient therapy ineffective. Furthermore, exogenous coagulation factors may only be administered intravenously, which is of considerable inconvenience and discomfort to patients. For example, infants and toddlers may have to have intravenous catheters surgically inserted into a chest vein, in order for venous access to be guaranteed. This leaves them at great risk of developing bacterial infections. Subjects with a coagulopathy may only receive therapy after a bleed has commenced, rather than as a precautionary measure, which often impinges upon their general quality of life.

Activation of the blood coagulation system relies on a complex cascade of biological reactions. When a vessel wall is injured, tissue factor (TF) is exposed to the contents of circulating blood and TF forms a complex with Factor VII/activated Factor VII (FVII/FVIIa) on the surface of TF-bearing cells. This leads to the activation of Factor X (FX) to FXa which together with FVa generates a limited amount of thrombin (FIIa). Small amounts of thrombin activate platelets, which results in surface exposure of phospholipids that supports the binding of the tenase complex consisting of activated FVIII:FIX (FVIIIa/FIXa).

The tenase complex produces large amounts of FXa, which subsequently facilitates a full thrombin burst. A full thrombin burst is needed for the formation of a mechanically strong fibrin structure and stabilization of the haemostatic plug. FVIII or FIX is missing or present at low levels in haemophilia A and B patients, respectively, and due to the resulting lack of tenase activity, the capacity to generate FXa is low and insufficient to support the propagation phase of coagulation. In contrast, the TF-mediated initiation phase is not dependent on the formation of the tenase complex. However, the TF-pathway will, shortly after an initial FXa generation, be blocked by plasma inhibitors.

Despite being downstream of the tenase complex, which is deficient in haemophilia, several in vivo studies in knock-out models have demonstrated a significant ameliorating effect of increased FVa levels. Approaches pursued to increase FVa levels include direct supplementation of exogenous FVa or interference with FVa inactivation by activated protein C (APC).

Thrombin generation is heavily regulated and one of the keys to the down regulation is the inactivation of FVa and FVIIIa. These molecules are inactivated by APC by proteolytic cleavages. The inactivation rate of both FVa and FVIIIa is increased by Protein S which is a cofactor for APC. It has been shown that the APC/Protein S complex reduces the lifetime of both FVa/FXa prothrombinase and the FVIIIa-FIXa tenase complex.

Blocking of Protein S binding to APC down-regulates the anti-coagulant potential of APC in normal plasma (Dahlback et al. JBC (1990) 265, 8127-35; He et al. Eur. J. Biochem (1995) 227, 433-40; Giri et al. Thromb. Haemost (1998) 80, 798-804; Stenberg et al. Eur. J. Biochem (1998) 251, 558-64; Hackeng et al. Biochem J. (2000) 349, 757-64; T. K. Giri, 2002, ISBN: 91-628-4164-5); Mille-Baker et al. Blood (2003) 101, 1416-8; Baroni et al. Thromb. Res. (2010) 125, e33-9; Andersson et al. Blood (2010) 115, 4878-85).

However, severe thromboembolic disease has been observed in individuals with homozygous deficiency of Protein S and a heterozygous Protein S deficiency has been shown to lead to a high incidence of thrombosis in persons with an otherwise normal coagulation system (Marlar & Neumann, Semin Thromb Hemost. (1990) 16:299-309; Schwarz et al., Blood (1984) 64:1297-1300). Similar observations have been made in murine models (Burstyn-Cohen et al., J Clin Invest. (2009) 119:2942-2953).

Protein S comprises five distinct structural domains; an N-terminal gamma-carboxylation (Gla) domain and aromatic stack, a so-called “thrombin-sensitive region” (TSR), four epidermal growth factor (EGF)-like domains (EGF1-4), and a large C-terminal region referred to as a sex-hormone binding globulin (SHBG)-like domain.

Dahlbäck et al. (1990) discloses experiments where several Ca²⁺-dependent monoclonal antibodies with undisclosed sequences were raised against Protein S, hereunder antibodies assumed to bind in the Gla domain, thrombin sensitive region and EGF1 or EGF2 domains of Protein S. These antibodies (undefined in terms of sequence) were used to investigate the APC cofactor activity of protein S in normal, i.e., non-haemophilic, plasma.

The SHBG-like domain is reported to be indispensable for expression of full cofactor activity in APC-catalysed inactivation of FVa and FVIIIa (Evenäs et al., Thromb Haemost (2000) 84:271-277; Nyberg et al., FEBS Lett (1998) 433:28-32).

Anti-Protein S monoclonal antibodies for non-medicinal in vitro purposes are commercially available (for examples hereof cf. table 3).

In a recent abstract Bologna et al. stated to have provided the first evidence that blocking Protein S has the ability to ameliorate haemophilia A as judged by in vivo improvement of bleeding phenotype in the tail clipping assay as well as in the acute hemarthrosis model (Bologna et al. Abstract, Blood; Nov. 15, 2013; 122 (21)).

Disclosed herein are novel anti-Protein S inhibitors in the form of antibodies with novel characteristics and uses. These antibodies are suitable for the development of pharmaceuticals. Such antibodies may have a substantial impact upon the quality of life of individuals suffering from a form of coagulopathy such as haemophilia.

### SUMMARY

The present invention relates to inhibitors which modulate Protein S activity and therapeutic uses thereof.

In particular the present invention relates to monoclonal antibodies or antigen-binding fragments thereof which specifically bind to Protein S and therapeutic uses thereof and to other related antibodies that are derived from these antibodies or have similar binding properties to these antibodies.

The invention also provides polynucleotides which encode an antibody of the invention, such as polynucleotides which encode an antibody light chain and/or an antibody heavy chain of the invention. Cells carrying such polynucleotides are also comprised by the invention.

The invention also provides pharmaceutical compositions comprising an antibody or polynucleotide of the invention and a pharmaceutically acceptable carrier.

The antibodies, polynucleotides and compositions of the invention are also provided for use in (a) the treatment or prevention of a coagulopathy (bleeding disorder) or (b) the stimulation of blood clotting. That is, the invention provides a method for (a) the treatment or prevention of a coagulopathy (bleeding disorder) or (b) the stimulation of blood clotting, the method comprising administering to a patient in need thereof a therapeutically or prophylactically effective amount of an antibody, polynucleotide or composition of the invention.

The antibodies, polynucleotides and compositions of the invention may be particularly useful in the treatment of haemophilia A and B with or without inhibitors. In one embodiment an antibody or antigen-binding fragment thereof of the invention may be capable of binding an epitope comprising amino acid residues W36, E39 and K40 and one or more of C41, E42 and F43 of SEQ ID NO: 2.

In certain embodiments an antibody or antigen-binding fragment thereof of the invention may have one or more of the following CDR sequences within the light chain: RASSSVSYMY (CDR1 residues 24-33 of SEQ ID NO: 49), ATSNLAS (CDR2 residues 49-55 of SEQ ID NO: 49) and QQWSSIPPT (CDR3 residues 88-96 of SEQ ID NO: 49).

In certain embodiments an antibody or antigen-binding fragment thereof of the invention may have one or more of the following CDR sequences within the heavy chain: SYWIN (CDR1 residues 31-35 of SEQ ID NO: 50), RIDPYDSETHYAQKFQG (CDR2 residues 50-66 of SEQ ID NO: 50) and WGGSGYAMDY (CDR3 residues 99-108 of SEQ ID NO: 50).

In certain embodiments an antibody or antigen-binding fragment thereof of the invention may comprise the light chain variable region SEQ ID NO: 49,

wherein amino acid residue L45 is substituted with P, and optionally

L46 is substituted with W.

and

a heavy chain variable region which comprises SEQ ID NO: 50, said heavy chain variable region optionally further comprising one or more of the substitutions selected from a group consisting of M70L, R72V, T74K and V79A.

### BRIEF DESCRIPTION OF DRAWINGS

FIG. 1: Anti-Protein S concentration dependent pro-coagulant effect in plasma from a person with severe haemophilia A

Polyclonal anti-Protein S antibodies concentration-dependently reduced clotting times in the presence of APC in FVIII deficient human plasma.

FIG. 2: In vitro display of maximal pro-coagulant effect in congenital human haemophilia A plasma

Maximal pro-coagulant effect in congenital human haemophilia A plasma obtained with DAKO anti-Protein S is comparable to effect obtained with 5-10% FVIII.

Column 1: HA plasma, 2: 1% FVIII, 3: 5% FVIII, 4: 10% FVIII, 5: NHP, 6: 1 mg/ml anti-Protein S antibody, 7: Protein S deficient plasma+anti-FVIII.

Data are mean±SD, n=3 experiments. HA: Haemophilia A, NHP: Normal Human Plasma, ProS: Protein S. ULOD: Upper Limit Of Detection.

FIG. 3: In vivo effect of polyclonal antibodies against full-length and Gla-domain deleted mouse Protein S

Haemophilia A mice treated with a rabbit polyclonal antibody against full length and desGla-domain mouse Protein S (49 mg/kg, IV), respectively, 5 min before tail clip (4 mm).

FIG. 4: In vitro effect of monoclonal antibodies in haemophilia A patient plasma

Effect of increasing concentrations of antibodies on thrombin generation parameter peak thrombin in severe haemophilia A patient platelet-poor plasma in the presence of 2 nM Activated Protein C (APC) (dotted line). Thrombin generation was triggered with 5 pM tissue-factor in the presence of 4 μM phospholipid and the amount of thrombin generated was estimated based on continuous reading of fluorescence generated by thrombin's conversion of FluCa reagent (Thrombinoscope, #TS50.00).

FIG. 5: In vitro effect of monoclonal antibodies on thrombin generation in normal and haemophilia A patient plasma

(A) Thrombin generation in platelet-poor normal human plasma (closed circles) or severe haemophilia A patient plasma added either buffer (open circles), 63 nM monoclonal antibody (mAb) 0910 (open triangles), 160 nM mAb 0910 (closed triangles), 63 nM mAb 0914 (open squares) or 160 nM mAb 0914 (closed squares). (B) Thrombin generation in platelet-poor severe haemophilia A patient plasma in the absence (closed circles) or presence of 5 nM Activated Protein C (APC) and either buffer (open circles), 63 nM mAb 0910 (open triangles), 160 nM mAb 0910 (closed triangles), 63 nM mAb 0914 (open squares) or 160 nM mAb 0914 (closed squares). (C-D) Effect of increasing concentrations of mAb 0910 (triangles) and mAb 0914 (squares) on thrombin generation parameter peak thrombin in severe haemophilia A patient plasma in (C) the absence of activated protein C (APC), or (D) in the presence of 5 nM APC (D). In all graphs thrombin generation was triggered with 5 pM tissue-factor in the presence of 4 μM phospholipid.

FIG. 6: In vitro effect of monoclonal antibodies on thrombin generation in rabbit and cynomolgus plasma

Thrombin generation in rabbit and cynomolgus platelet poor plasma (diluted 1:3) in the present of thrombomodulin (50 nM) and increasing concentration (0 nM-1000 nM) of 0322-0000-0114 (mAb 0114) (A) and 0322-0000-0914 (mAb 0914) (B; n=3). Dose respond of 0322-0000-0910 (mAb 0910) (C) was only performed in cynomolgus monkey plasma (diluted 1:3). Thrombin generation was triggered with 5 pM tissue-factor in the presence of 4 μM phospholipid. The dotted line indicates the peak thrombin concentration without added TM for the individual experiment.

FIG. 7: SPR binding sensorgram

SPR sensorgrams for binding of monoclonal antibodies 0322-0000-0114 (mAb 0114) (solid line) and 0322-0000-0203 (mAb 0203) (dotted line) to Protein S captured on phosphatidylserine-containing lipid vesicles.

FIG. 8: SPR binding sensorgram

SPR sensorgrams for binding of free Protein S (100 nM) or Protein S (100 nM) incubated with monoclonal antibodies (500 nM) to phosphatidylserine-containing lipid vesicles.

FIGS. 9 and 10: CDR annotations

CDR annotations (CDR1s in bold, CDR2s in dark grey/green, CDR3s in light gray/cyan) for SEQ ID NOs: 4-45 of the (below description of) sequence listing.

FIG. 11: Effect of anti-Protein S mAb 0914 in a rabbit cuticle bleeding model with induced haemophilia A

Anti-Protein S mAb 0914 significantly reduced bleeding relative to an isotype control antibody (p=0.013).

FIG. 12: The activity of FXa alone or in the presence of Protein S and mAbs

The activity of FXa alone or in the presence of Protein S and mAbs is followed over time by measuring hydrolysis of a small chromogenic substrate, S-2765. FXa alone (black solid line), with TFPI (Black dashed line), with TFPI/Protein S (grey solid line); with TFPI/Protein S/-1069 (grey dashed line), with TFPI/Protein S/-1139 (grey dotted line).

FIG. 13: Binding of free Protein S in complex with mAb to human TFPI

Binding of free protein (black solid line) or Protein S in complex with 0322-0000-1069 (black dotted line), 0322-0000-1139 (black dashed line), or 0322-0000-0023 (grey solid line) to human TFPI.

FIG. 14: Humanization model for the 0322-0000-0914 light chain variable domain (VL)

Potential back mutations derived from the humanization protocol as described in example 22 are highlighted in grey. CDRs 1, 2 and 3 as defined by Kabat are shown in bold and underlined.

FIG. 15: Humanization model for the 0322-0000-0914 heavy chain variable domain (VH)

Potential back mutations derived from the humanization protocol as described in example 22 are highlighted in grey. CDRs 1, 2 and 3 as defined by Kabat are shown in bold and underlined.

### BRIEF DESCRIPTION OF THE SEQUENCE LISTING

SEQ ID NO: 1 gives the amino acid sequence of desGla human Protein S. The N-terminus of the truncated protein corresponds to N-terminal beginning of the EGF1 region. Residues 564-578 in the listed sequence, represent a cloning spacer (ALA) followed by an HPC4 purification tag (EDQV DPRLIDGK).

SEQ ID NO: 2 gives the amino acid sequence of the EGF1-4 domains of human Protein S. Residues 174-188 in the listed sequence, represent a cloning spacer (ALA) followed by an HPC4 purification tag (EDQVDPRLIDGK).

SEQ ID NO: 3 gives the amino acid sequence of Macaca fascicularis Protein S. Residues 636-647 in the listed sequence, represent an HPC4 purification tag (EDQV DPRLIDGK).

SEQ ID NO: 4 and 5 gives the amino acid sequences of the light chain variable domain (VL) and heavy chain variable domain (VH) of the M-hProtS-2F188A1 monoclonal antibody, respectively.

SEQ ID NO: 6 and 7 gives the amino acid sequences of the light chain variable domain (VL) and heavy chain variable domain (VH) of the M-hProtS-2F380A1 monoclonal antibody, respectively.

SEQ ID NO: 8 and 9 gives the amino acid sequences of the light chain variable domain (VL) and heavy chain variable domain (VH) of the M-hProtS-2F382A1 monoclonal antibody, respectively.

SEQ ID NO: 10 and 11 gives the amino acid sequences of the light chain variable domain (VL) and heavy chain variable domain (VH) of the M-hProtS-2F4A1 monoclonal antibody, respectively.

SEQ ID NO: 12 and 13 gives the amino acid sequences of the light chain variable domain (VL) and heavy chain variable domain (VH) of the M-hProtS-2F82A1 monoclonal antibody, respectively.

SEQ ID NO: 14 and 15 gives the amino acid sequences of the light chain variable domain (VL) and heavy chain variable domain (VH) of the M-hProtS-3F2A1 monoclonal antibody, respectively.

SEQ ID NO: 16 and 17 gives the amino acid sequences of the light chain variable domain (VL) and heavy chain variable domain (VH) of the M-hProtS-3F38A2 monoclonal antibody, respectively.

SEQ ID NO: 18 and 19 gives the amino acid sequences of the light chain variable domain (VL) and heavy chain variable domain (VH) of the M-hProtS-3F62A5 monoclonal antibody, respectively.

SEQ ID NO: 20 and 21 gives the amino acid sequences of the light chain variable domain (VL) and heavy chain variable domain (VH) of the M-hProtS-6F101A3 monoclonal antibody, respectively.

SEQ ID NO: 22 and 23 gives the amino acid sequences of the light chain variable domain (VL) and heavy chain variable domain (VH) of the M-hProtS-6F120A1 monoclonal antibody, respectively.

SEQ ID NO: 24 and 25 gives the amino acid sequences of the light chain variable domain (VL) and heavy chain variable domain (VH) of the M-hProtS-6F128A2 monoclonal antibody, respectively.

SEQ ID NO: 26 and 27 gives the amino acid sequences of the light chain variable domain (VL) and heavy chain variable domain (VH) of the M-hProtS-6F138A3 monoclonal antibody, respectively.

SEQ ID NO: 28 and 29 gives the amino acid sequences of the light chain variable domain (VL) and heavy chain variable domain (VH) of the M-hProtS-6F145A11 monoclonal antibody, respectively.

SEQ ID NO: 30 and 31 gives the amino acid sequences of the light chain variable domain (VL) and heavy chain variable domain (VH) of the M-hProtS-6F151A2 monoclonal antibody, respectively.

SEQ ID NO: 32 and 33 gives the amino acid sequences of the light chain variable domain (VL) and heavy chain variable domain (VH) of the M-hProtS-6F153A2 monoclonal antibody, respectively.

SEQ ID NO: 34 and 35 gives the amino acid sequences of the light chain variable domain (VL) and heavy chain variable domain (VH) of the M-hProtS-6F159A11 monoclonal antibody, respectively.

SEQ ID NO: 36 and 37 gives the amino acid sequences of the light chain variable domain (VL) and heavy chain variable domain (VH) of the M-hProtS-6F170A2 monoclonal antibody, respectively.

SEQ ID NO: 38 and 39 gives the amino acid sequences of the light chain variable domain (VL) and heavy chain variable domain (VH) of the M-hProtS-6F206A1 monoclonal antibody, respectively.

SEQ ID NO: 40 and 41 gives the amino acid sequences of the light chain variable domain (VL) and heavy chain variable domain (VH) of the M-hProtS-6F216A3 (mAb 0914) monoclonal antibody, respectively.

SEQ ID NO: 42 and 43 gives the amino acid sequences of the light chain variable domain (VL) and heavy chain variable domain (VH) of the M-hProtS-6F230A10 monoclonal antibody, respectively.

SEQ ID NO: 44 and 45 gives the amino acid sequences of the light chain variable domain (VL) and heavy chain variable domain (VH) of the M-hProtS-6F265A1 monoclonal antibody, respectively.

SEQ ID NO: 46 gives the amino acid sequence of human Protein S (signal peptide included).

SEQ ID NO: 47 gives the reverse primer sequence used for HC (VH domain) amplification.

SEQ ID NO: 48 gives the reverse primer sequence used for LC amplification.

SEQ ID NO: 49 gives the amino acid sequences of the light chain variable domain (VL) of the humanized monoclonal antibody 0322-0000-1152.

SEQ ID NO: 50 gives the amino acid sequences of the heavy chain variable domain (VH) of the humanized monoclonal antibodies: 0322-0000-1152, 0322-0000-1166 and 0322-0000-1223.

SEQ ID NO: 51 gives the amino acid sequences of the light chain variable domain (VL) of the humanized monoclonal antibodies: 0322-0000-1166, 0322-0000-1201, 0322-0000-1238 and 0322-0000-1239.

SEQ ID NO: 52 gives the amino acid sequences of the heavy chain variable domain (VH) of the humanized monoclonal antibodies: 0322-0000-1201 and 0322-0000-1246.

SEQ ID NO: 53 gives the amino acid sequences of the light chain variable domain (VL) of the humanized monoclonal antibodies: 0322-0000-1223, 0322-0000-1246, 0322-0000-1248 and 0322-0000-1249.

SEQ ID NO: 54 gives the amino acid sequences of the heavy chain variable domain (VH) of the humanized monoclonal antibodies: 0322-0000-1238 and 0322-0000-1248.

SEQ ID NO: 55 gives the amino acid sequences of the heavy chain variable domain (VH) of the humanized monoclonal antibodies: 0322-0000-1239 and 0322-0000-1249.

SEQ ID NO: 56 gives the amino acid sequences of the light chain (LC) of the humanized monoclonal antibody 0322-0000-1152.

SEQ ID NO: 57 gives the amino acid sequences of the heavy chain (HC) of the humanized monoclonal antibodies: 0322-0000-1152, 0322-0000-1166 and 0322-0000-1223.

SEQ ID NO: 58 gives the amino acid sequences of the light chain (LC) of the humanized monoclonal antibodies: 0322-0000-1166, 0322-0000-1201, 0322-0000-1238 and 0322-0000-1239.

SEQ ID NO: 59 gives the amino acid sequences of the heavy chain (HC) of the humanized monoclonal antibodies: 0322-0000-1201 and 0322-0000-1246.

SEQ ID NO: 60 gives the amino acid sequences of the light chain (LC) of the humanized monoclonal antibodies: 0322-0000-1223, 0322-0000-1246, 0322-0000-1248 and 0322-0000-1249.

SEQ ID NO: 61 gives the amino acid sequences of the heavy chain (HC) of the humanized monoclonal antibodies: 0322-0000-1238 and 0322-0000-1248.

SEQ ID NO: 62 gives the amino acid sequences of the heavy chain (HC) of the humanized monoclonal antibodies: 0322-0000-1239 and 0322-0000-1249.

A table linking the names and IDs of hybridomas, recombinantly expressed mouse IgG1 antibodies and recombinantly expressed murine-human chimeric antibodies with SEQ ID NOs are included in example 25 (table 15).

### DETAILED DESCRIPTION

The present invention relates to pro-coagulant inhibitors that modulate Protein S activity. The invention also relates to uses for such inhibitors, such as therapeutic and pharmaceutical uses. The present invention also relates to polynucleotides optionally incorporated into a vector which encode said inhibitor.

In some embodiments, the inhibitor provides an on-demand or prophylactic treatment option for patients suffering from a coagulopathy.

In some embodiments, the inhibitor provides an on-demand or prophylactic treatment option for haemophilia patients with or without inhibitors.

In some embodiments, the inhibitor is a monoclonal antibody or antigen-binding fragment thereof that modulates Protein S activity.

In some embodiments, the inhibitor is an antibody or antigen-binding fragment thereof capable of inhibiting the anti-coagulant effect of Protein S.

In some embodiments, antibodies or antigen-binding fragments thereof described herein provide an on-demand or prophylactic treatment option for patients suffering from a coagulopathy.

The present invention also provides a method for treatment of haemophilia patients in a FVIII and FIX independent manner. Hence, in some embodiments, antibodies described herein provide an on-demand or prophylactic treatment option for haemophilia A and B patients with or without inhibitors.

In one embodiment polyclonal antibodies raised against human Protein S significantly improve the clot time in an activated partial thromboplastin time (APTT) assay in haemophilic plasma.

In one embodiment polyclonal anti-Protein S antibodies raised against murine Protein S significantly reduced the blood loss in the tail bleeding model in haemophilic mice.

In one embodiment a monoclonal anti-Protein S antibody has been shown to significantly reduce blood loss in vivo in a rabbit haemophilia model.

In one embodiment monoclonal anti-Protein S antibodies have been found to be capable of increasing thrombin generation in human haemophilia A (HA) (FVIII deficient) plasma.

Antibodies can be administered subcutaneously and thus significantly reduce the burden of treatment as compared to the treatment options currently on the market.

Hence, the antibodies, other molecules and compositions of the present invention have numerous in vitro and in vivo therapeutic utilities involving the treatment and prevention of clotting related disorders. For example, these antibodies and compositions can be administered to human subjects to prevent or treat a variety of disorders.

In particular, the present invention provides methods for the treatment of bleeding disorders or for the enhancement of blood clotting comprising administering to a patient in need thereof an effective amount of an antibody or other molecule or composition of the invention. For example, such methods may be for the treatment of clotting factor deficiencies such as haemophilia A, haemophilia B, Factor XI deficiency, Factor VII deficiency, thrombocytopenia or von Willebrand's disease. Such methods may be for the treatment of conditions accompanied by the presence of a clotting factor inhibitor. Such methods may be for the treatment of excessive bleeding. The antibodies and compositions of the invention may be used to treat patients before, during, or after surgery or anticoagulant therapy or after trauma. The antibodies and compositions described herein may be used in any such treatment or may be used in the manufacture of a medicament for use in any such treatment.

In some therapeutic applications, antibodies or compositions are administered to a subject already suffering from a disorder or condition as described above, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”. For example, where the treatment is for unwanted bleeding, therapy may be defined as a decrease in the amount of bleeding or suitable coagulation to stop the bleeding altogether.

In prophylactic or preventive applications, antibodies or compositions are administered to a subject at risk of a disorder or condition as described above, in an amount sufficient to prevent or reduce the subsequent effects of the condition or one or more of its symptoms. An amount adequate to accomplish this is defined as a “prophylactically effective amount”. For example, where the treatment is to prevent unwanted bleeding, a prophylactic effect may be defined as the prevention of bleeding or a reduced period or quantity of bleeding compared to that that would be seen in the absence of the modulator.

Effective amounts for each purpose will depend on the severity of the disease or injury as well as the weight and general state of the subject.

### Coagulopathy/Haemophilia

In subjects with a coagulopathy, such as in human beings with haemophilia A and B, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding, or damage to internal organs, such as the joints. Individuals with haemophilia A and B may receive coagulation factor replacement therapy such as exogenous FVIII or FIX, respectively. However, such patients are at risk of developing neutralizing antibodies, so-called “inhibitors”, to such exogenous factors, rendering formerly efficient therapy ineffective.

Furthermore, exogenous coagulation factors may only be administered intravenously, which is of considerable inconvenience and discomfort to patients. For example, infants and toddlers may have to have intravenous catheters surgically inserted into a chest vein, in order for venous access to be guaranteed. This leaves them at great risk of developing bacterial infections. Subjects with a coagulopathy may only receive therapy after a bleed has commenced, rather than as a precautionary measure, which often impinges upon their general quality of life.

Currently, the gold standard in treatment of haemophilia is prophylactic replacement therapy, wherein treatment has to be administered intravenously 2-3 times weekly or modified variants wherein treatment has to be administered intravenously every 7-10 day or every fourth day for FIX and FVIII variants, respectively causing a significant burden to the patient Furthermore, approximately 30% of the patients treated with e.g. FVIII develop inhibitors which reduce the possibilities for an effective prophylactic treatment.

The term “subject”, as used herein, includes any human patient, or non-human vertebrate.

The term “coagulopathy”, as used herein, refers to an increased haemorrhagic tendency which may be caused by any qualitative or quantitative deficiency of any pro-coagulative component of the normal coagulation cascade, or any upregulation of fibrinolysis. Such coagulopathies may be congenital and/or acquired and/or iatrogenic and are identified by a person skilled in the art.

Non-limiting examples of congenital hypocoagulopathies are haemophilia A, haemophilia B, Factor VII deficiency, Factor X deficiency, Factor XI deficiency, von Willebrand's disease and thrombocytopenias such as Glanzmann's thombasthenia and Bernard-Soulier syndrome. Said haemophilia A or B may be severe, moderate or mild. The clinical severity of haemophilia is determined by the concentration of functional units of FIX/FVIII in the blood and is classified as mild, moderate, or severe. Severe haemophilia is defined by a clotting factor level of <0.01 U/ml corresponding to <1% of the normal level, while moderate and mild patients have levels from 1-5% and >5%, respectively.

Haemophilia A with “inhibitors” (that is, allo-antibodies against Factor VIII) and haemophilia B with “inhibitors” (that is, allo-antibodies against Factor IX) are non-limiting examples of coagulopathies that are partly congenital and partly acquired.

A non-limiting example of an acquired coagulopathy is serine protease deficiency caused by vitamin K deficiency; such vitamin K-deficiency may be caused by administration of a vitamin K antagonist, such as warfarin. Acquired coagulopathy may also occur following extensive trauma. In this case otherwise known as the “bloody vicious cycle”, it is characterised by haemodilution (dilutional thrombocytopaenia and dilution of clotting factors), hypothermia, consumption of clotting factors and metabolic derangements (acidosis). Fluid therapy and increased fibrinolysis may exacerbate this situation. Said haemorrhage may be from any part of the body.

A non-limiting example of an iatrogenic coagulopathy is an over dosage of anticoagulant medication—such as heparin, aspirin, warfarin and other platelet aggregation inhibitors—that may be prescribed to treat thromboembolic disease. A second, non-limiting example of iatrogenic coagulopathy is that which is induced by excessive and/or inappropriate fluid therapy, such as that which may be induced by a blood transfusion.

In one embodiment of the current invention, haemorrhage is associated with haemophilia A or B. In another embodiment, haemorrhage is associated with haemophilia A or B with acquired inhibitors. In another embodiment, haemorrhage is associated with thrombocytopenia. In another embodiment, haemorrhage is associated with von Willebrand's disease. In another embodiment, haemorrhage is associated with severe tissue damage. In another embodiment, haemorrhage is associated with severe trauma. In another embodiment, haemorrhage is associated with surgery. In another embodiment, haemorrhage is associated with haemorrhagic gastritis and/or enteritis. In another embodiment, the haemorrhage is profuse uterine bleeding, such as in placental abruption. In another embodiment, haemorrhage occurs in organs with a limited possibility for mechanical haemostasis, such as intracranially, intraaurally or intraocularly. In another embodiment, haemorrhage is associated with anticoagulant therapy.

The term “treatment”, as used herein, refers to the medical therapy of any human or other animal subject in need thereof. Said subject may have undergone physical examination by a medical practitioner, who has given a tentative or definitive diagnosis which would indicate that the use of said specific treatment is beneficial to the health of said human or other animal subject. The timing and purpose of said treatment may vary from one individual to another, according to the status quo of the subject's health.

Thus, said treatment may be prophylactic, palliative and/or symptomatic. In terms of the present invention, prophylactic, palliative and symptomatic may represent separate aspects of the invention.

### Protein S

Protein S is a vitamin K-dependent plasma glycoprotein with a molecular weight of approximately 70 kDa synthesized predominantly within the liver. However, a significant amount is also synthesized in endothelial cells. Mature Protein S comprises five distinct structural domains, including an N-terminal gamma-carboxylation (Gla) domain (residues 1-37) and aromatic stack (residues 38-45), a so-called “thrombin-sensitive region” (TSR; residues 46-74), 4 EGF-like domains [EGF1 (residues 75-115), EGF2 (residues 116-159), EGF3 (residues 160-201) and EGF4 (residues 202-242)], and a large C-terminal region of 393 amino acids referred to as a sex-hormone binding globulin (SHBG)-like domain (residues 243-635) the structure of which represents two laminin G-type domains.

The plasma concentration of Protein S is ˜350 nM and roughly 60% is bound to the complement 4 binding protein (C4b-BP), while the remaining fraction circulates as “free” Protein S. The complex bound Protein S has approximately 40% anti-coagulant activity compared to that of free Protein S. The half-life in plasma is 48-60 hours. Site-directed mutagenesis of Protein S has been used to determine the interaction site for APC. The studies show that the APC binding sites are located in the Gla domain, the TSR, and the EGF1 and EGF2 domains of Protein S. However, studies have also suggested that the EGF3-4 domains may be involved, and it remains unknown whether one binding site may be the dominant interaction site for APC.

The SHBG-like domain is reported to be indispensable for expression of full cofactor activity in APC-catalysed inactivation of FVa and FVIIIa (Evenäs et al., Thromb Haemost (2000) 84:271-277; Nyberg et al., FEBS Lett (1998) 433:28-32).

In addition to the anti-coagulant function, Protein S also plays a role in other processes. Thus, Protein S has been described to mediate the clearance of apoptotic cells, to be neuroprotective in mice and to be an endogenous inhibitor of angiogenesis.

One of the keys to the regulation of thrombin is the inactivation of Factor Va by APC and its cofactor Protein S.

Generation of a thrombin burst is central for the generation of a stable clot after injury to the vessel wall. Key to the production of thrombin is Factor Xa and its cofactor Factor Va. This complex generates both the initial small amount of thrombin required for the first activation of platelets during the initiation phase of the coagulation process and the thrombin burst on the activated platelets during the coagulation propagation phase where large amounts of FXa is generated by the Factor VIIIa:Factor IXa complex.

In patients with haemophilia A of B the propagation phase cannot take place and consequently insufficient thrombin is generated to form a clot.

An alternative treatment could be to exclusively augment the generation of thrombin in the initiation phase of the coagulation. Thrombin generation is heavily regulated and one of the keys to the down regulation is the inactivation of Factor Va. Factor Va is inactivated by APC by proteolytic cleavages at Arg 506 and Arg 306. In vitro, the cleavages of Arg 506 is kinetically favoured and yields a Factor Va with approximately 40% Factor Xa cofactor activity while cleavages of Arg 306 result in almost complete inactivation of Factor Va. The inactivation rate of Factor Va is increased by Protein S which is a cofactor for APC. Thus, cleavage at Arg 506 is increased 5-fold whereas the cleavage at Arg 306 is increased approximately 20-fold.

However, severe thromboembolic disease has been observed in individuals with homozygous deficiency of Protein S and a heterozygous Protein S deficiency has been shown to lead to a high incidence of thrombosis in persons with an otherwise normal coagulation system (Marlar & Neumann, Semin Thromb Hemost. (1990) 16:299-309; Schwarz et al., Blood (1984) 64:1297-1300). Similar observations have been made in murine models (Burstyn-Cohen et al., J Clin Invest. (2009) 119:2942-2953).

### Coagulation Factors

### Factor V

Factor V (FV) is synthesized by the liver and secreted FV circulates in plasma as a 330-kDa single-chain polypeptide that is the inactive procoagulant. FV consists of 2196 amino acids, including a 28 amino acids signal peptide. It is composed of six domains A1 (Aa 30-329), A2 (Aa 348-684), B (Aa 692-1573), A3 (Aa 1578-1907), C1 (Aa 1907-2061), and C2 (Aa 2066-2221). The A and C domains of the two proteins are approximately 40% homologous with the equivalent domains of FVIII, but the B domains are not conserved. As is the case with FVIII, FV activity is tightly regulated via site-specific proteolysis. Thrombin, and to a lesser extent Factor Xa (FXa), are primarily responsible for FV activation via proteolytic cleavages at positions Arg⁷⁰⁹-Ser⁷¹⁰, Arg¹⁰¹⁸-Thr¹⁰¹⁹ and Arg¹⁵⁴⁵-Ser¹⁵⁴⁶. These cleavages release the B domain and create a dimeric molecule composed of a 105-kDa heavy chain that contains the A1 and A2 domains and a 71- to 74-kDa light chain that contains the A3, C1, and C2 domains. These two chains are held together by calcium at residues Asp¹³⁹ and Asp¹⁴⁰ and hydrophobic interactions. The heavy chain provides the contacts for both FXa and Prothrombin, whereas the two C domains in the light chain are needed for the interaction of FVa with the phospholipid surface.

Thus, FV is active as a cofactor for FXa of the thrombinase complex and the activated FXa enzyme requires calcium and FVa to convert prothrombin to thrombin on the cell surface membrane. The A3 domain in the light chain is involved in both FXa and phospholipid interactions. Taken together, the two FVa chains link FXa to the phospholipid surface formed by the platelet plug at the site of injury and enable FXa to efficiently bind and cleave prothrombin to generate thrombin. FV is able to bind to activated platelets. Although FV is predominately found as a soluble component in blood plasma, a fraction of FV is also present in the α-granula of platelets, which is important for normal hemostasis as evidenced by platelet specific FV deficiency.

### Factor VIII

Factor VIII (FVIII) is a large, complex glycoprotein that is primarily produced by hepatocytes. FVIII consists of 2351 amino acids, including a signal peptide, and contains several distinct domains as defined by homology. There are three A-domains, a unique B-domain, and two C-domains. The domain order can be listed as NH2-A1-A2-B-A3-C1-C2-COOH. FVIII circulates in plasma as two chains, separated at the B-A3 border. The chains are connected by bivalent metal ion-bindings. The A1-A2-B chain is termed the heavy chain (HC) while the A3-C1-C2 is termed the light chain (LC). Small acidic regions C-terminal of the A1 (the a1 region) and A2 (the a2 region) and N-terminal of the A3 domain (the a3 region) play important roles in its interaction with other coagulation proteins, including thrombin and von Willebrand factor (vWF or VWF), the carrier protein for FVIII. Endogenous FVIII molecules circulate in vivo as a pool of molecules with B domains of various sizes, the shortest having C-terminal at position 740, i.e., at the C-terminal of A2-a2. These FVIII molecules with B-domains of different length all have full procoagulant activity. Upon activation with thrombin, FVIII is cloven C-terminal of A1-a1 at position 372, C-terminal of A2-a2 at position 740, and between a3 and A3 at position 1689, the latter cleavage releasing the a3 region with concomitant loss of affinity for VWF. The activated FVIII molecule is termed FVIIIa. The activation allows interaction of FVIIIa with phospholipid surfaces like activated platelets and activated Factor IX (FIXa), i.e., the tenase complex is formed, allowing efficient activation of Factor X (FX).

The B domain is cloven at several different sites, generating large heterogeneity in circulating plasma FVIII molecules. The exact function of the heavily glycosylated B domain is unknown.

### Factor IX

FIX is a vitamin K-dependent coagulation factor with structural similarities to Factor VII, prothrombin, Factor X, and Protein C. The circulating zymogen form consists of 415 amino acids divided into four distinct domains comprising an N-terminal γ-carboxyglutamic acid-rich (Gla) domain, two EGF domains and a C-terminal trypsin-like serine protease domain.

Activation of FIX occurs by limited proteolysis at Arg¹⁴⁵-Ala¹⁴⁶ and Arg¹⁸⁰-Val¹⁸¹ releasing a 35-aa fragment, the so-called activation peptide. The activation peptide is heavily glycosylated, containing two N-linked and up to four O-linked glycans. Activated Factor IX is referred to as Factor IX(a) or FIX(a). FIX(a) is a trypsin-like serine protease that serves a key role in haemostasis by generating, as part of the tenase complex, most of the Factor Xa required to support proper thrombin formation during coagulation.

Unless otherwise specified, FIX domains include the following amino acid residues: Gla domain being the region from reside Tyr1 to residue Lys43; EGF1 being the region from residue Gln44 to residue Leu84; EGF2 being the region from residue Asp85 to residue Arg145; the Activation Peptide being the region from residue Ala146 to residue Arg180; and the Protease Domain being the region from residue Va1181 to Thr414. The light chain refers to the region encompassing the Gla domain, EGF1 and EGF2, while the heavy chain refers to the Protease Domain.

### Factor X

Coagulation Factor X (FX) is a vitamin K-dependent coagulation factor with structural similarities to Factor VII, prothrombin, Factor IX (FIX), and protein C. It is synthesised with a 40-residue pre-pro-sequence containing a hydrophobic signal sequence (Aa1-31) that targets the protein for secretion. The pro-peptide is important for directing y-carboxylation to the light chain of Factor X. The circulating human FX zymogen consists of 445 amino acids divided into four distinct domains comprising an N-terminal gamma-carboxyglutamic acid rich (Gla) domain, two EGF domains, and a C-terminal trypsin-like serine protease domain. The mature two-chain form of FX consists of a light chain (Aa41-179) and a heavy chain (Aa183-488) held together by a disulfide bridge (Cys¹⁷²-Cys³⁴²) and by an Arg-Lys-Arg (RKR) tripeptide. The light chain contains 11 Gla residues, which are important for Ca²⁺-dependent binding of FX to negatively charged phospholipid membranes. Wild-type human coagulation Factor X has two N-glycosylation sites (Asn²²¹ and Asn²³¹) and two O-glycosylation sites (Thr¹⁹⁹ and Thr²¹¹) in the activation peptide. β-hydroxylation occurs at Asp¹⁰³ in the first EGF domain, resulting in β-hydroxyaspartic acid (Hya). Activation of FX occurs by limited proteolysis at Arg²³⁴-Ile²³⁵ releasing a 52 amino acid activation peptide (Aa183-234). In the extrinsic pathway, this occurs upon exposure of Tissue Factor (TF) on the membrane of subendothelial cells to plasma and subsequent activation of FVIIa. Activation via the intrinsic pathway occurs with the interaction of FIXa, FVIIIa, calcium and acidic phospholipid surfaces. Prothrombin is the most important substrate of FXa, but the activation requires FXa's cofactor FVa, calcium and acidic phospholipid surface. FX deficiency is a rare autosomal recessive bleeding disorder with an incidence of 1:1,000,000 in the general population. Although it produces a variable bleeding tendency, patients with a severe FX deficiency tend to be the most seriously affected among patients with rare coagulation defects. The prevalence of heterozygous FX deficiency is about 1:500, but is usually clinically asymptomatic.

### Antibodies

The term “antibody” herein refers to a protein, derived from a germline immunoglobulin sequence, which is capable of specifically binding to an antigen or a portion thereof. The term antibody includes full length antibodies of any class (or isotype), that is, IgA, IgD, IgE, IgG, IgM and/or IgY. An antibody that specifically binds to an antigen, or portion thereof, may bind exclusively to that antigen, or portion thereof, or it may bind to a limited number of homologous antigens, or portions thereof.

Natural full-length antibodies usually comprise at least four polypeptide chains: two heavy (H) chains and two light (L) chains that are connected by disulfide bonds. In some cases, natural antibodies comprise less than four chains, as in the case of the heavy chain only antibodies found in camelids (V_{H}H fragments) and the IgNARs found in Chondrichthyes. One class of immunoglobulins of particular pharmaceutical interest is the IgGs. In humans, the IgG class may be sub-divided into four sub-classes IgG1, IgG2, IgG3 and IgG4, based on the sequence of their heavy chain constant regions. The light chains can be divided into two types, kappa and lambda chains based on differences in their sequence composition. IgG molecules are composed of two heavy chains, interlinked by two or more disulfide bonds, and two light chains, each attached to a heavy chain by a disulfide bond. An IgG heavy chain may comprise a heavy chain variable region (VH) and up to three heavy chain constant (CH) regions: CH1, CH2 and CH3. A light chain may comprise a light chain variable region (VL) and a light chain constant region (CL). VH and VL regions can be further subdivided into regions of hypervariability, termed complementarity determining regions (CDRs) or hypervariable regions (HvRs), interspersed with regions that are more conserved, termed framework regions (FR). VH and VL regions are typically composed of three CDRs and four FRs, arranged from amino-terminus to carboxy-terminus in the following order: FR1, CDR1, FR2, CDR2, FR3, CDR3, FR4. The variable domains with the hypervariable regions of the heavy and light chains form a domain that is capable of interacting with an antigen, whilst the constant region of an antibody may mediate binding of the immunoglobulin to host tissues or factors, including, but not limited to various cells of the immune system (effector cells), Fc receptors and the first component (C1q) of the C1 complex of the classical complement system.

Antibodies of the invention may be monoclonal antibodies, in the sense that they represent a set of unique heavy and light chain variable domain sequences as expressed from a single B-cell or by a clonal population of B cells. Antibodies of the invention may be produced and purified using various methods that are known to the person skilled in the art. For example, antibodies may be produced from hybridoma cells. Antibodies may be produced by B-cell expansion. Antibodies or fragments thereof may be recombinantly expressed in mammalian or microbial expression systems, or by in vitro translation.

Antibodies or fragments thereof may also be recombinantly expressed as cell surface bound molecules, by means of e.g. phage display, bacterial display, yeast display, mammalian cell display or ribosome or mRNA display.

Antibodies of the current invention may be isolated. The term “isolated antibody” refers to an antibody that has been separated and/or recovered from (an)other component(s) in the environment in which it was produced and/or that has been purified from a mixture of components present in the environment in which it was produced.

Certain antigen-binding fragments of antibodies may be suitable in the context of the current invention, as it has been shown that the antigen-binding function of an antibody can be performed by fragments of a full-length antibody.

The term “antigen-binding fragment” of an antibody refers to one or more fragment(s) of an antibody that retain(s) the ability to specifically bind to or recognise an antigen, such as the EGF1-4 region of Protein S as described herein. Examples of antigen-binding fragments include Fab, Fab′, F(ab)₂, F(ab′)₂, F(ab)S, Fv (typically the VL and VH domains of a single arm of an antibody), single-chain Fv (scFv; see e.g. Bird et al. Science (1988) 242:423-426; and Huston et al. PNAS (1988) 85:5879-5883), dsFv, Fd (typically the VH and CH1 domain), and dAb (typically a VH domain) fragments; VH, VL, VhH, and V-NAR domains; monovalent molecules comprising a single VH and a single VL chain; minibodies, diabodies, triabodies, tetrabodies, and kappa bodies (see, e.g., Ill et al. Protein Eng (1997) 10:949-57); camel IgG; IgNAR; as well as one or more isolated CDRs or a functional paratope, where the isolated CDRs or antigen-binding residues or polypeptides can be associated or linked together so as to form a functional antibody fragment. Various types of antibody fragments have been described or reviewed in, e.g., Holliger and Hudson, Nat Biotechnol (2005) 23:1126-1136; WO2005040219, and published U.S. Patent Applications 20050238646 and 20020161201. These antibody fragments may be obtained using conventional techniques known to those of skill in the art, and the fragments may be screened for utility in the same manner as intact antibodies.

“Fab fragments” of an antibody, including “Fab” and “F(ab′)₂” fragments, are derived from said antibody by cleavage of the heavy chain in the hinge region on the N-terminal or C-terminal side of the hinge cysteine residues connecting the heavy chains of the antibody. A Fab fragment includes the variable and constant domains of the light chain and the variable domain and the first constant domain (CH1) of the heavy chain. “F(ab′)₂” fragments comprise a pair of “Fab” fragments that are generally covalently linked by their hinge cysteines. A Fab′ is formally derived from a F(ab′)₂ fragment by cleavage of the hinge disulfide bonds connecting the heavy chains in the F(ab′)₂. Other chemical couplings than disulfide linkages of antibody fragments are also known in the art. A Fab fragment retains the ability of the parent antibody to bind to its antigen, potentially with a lower affinity. F(ab′)₂ fragments are capable of divalent binding, whereas Fab and Fab′ fragments can bind monovalently.

Generally, Fab fragments lack the constant CH2 and CH3 domains, i.e., the Fc part, where interaction with the Fc receptors would occur. Thus, Fab fragments are in general devoid of effector functions. Fab fragments may be produced by methods known in the art, either by enzymatic cleavage of an antibody, e.g. using papain to obtain the Fab or pepsin to obtain the F(ab′)₂, Fab fragments including Fab, Fab′, F(ab′)₂ may be produced recombinantly using techniques that are well known to the person skilled in the art.

An “Fv” fragment is an antibody fragment that contains a complete antigen recognition and binding site, and generally comprises a dimer of one heavy and one light chain variable domain in association that can be covalent in nature, for example in a single chain variable domain fragment (scFv). It is in this configuration that the three hypervariable regions of each variable domain interact to define an antigen-binding site on the surface of the VH-VL dimer. Collectively, the six hypervariable regions or a subset thereof confer antigen binding specificity to the antibody. However, even a single variable domain comprising only three hypervariable regions specific for an antigen can retain the ability to recognize and bind antigen, although usually at a lower affinity than the entire binding site (Cai & Garen, Proc. Natl. Acad. Sci. USA (1996) 93:6280-6285). For example, naturally occurring camelid antibodies that only have a heavy chain variable domain (VHH) can bind antigen (Desmyter et al. J. Biol. Chem. (2002) 277:23645-23650; Bond et al. J. Mol. Biol. (2003) 332:643-655).

“Single-chain Fv” or “scFv” antibody fragments comprise the VH and VL domains of antibody, where these domains are present in a single polypeptide chain. Generally, the Fv polypeptide further comprises a polypeptide linker between the VH and VL domains that enables the scFv to form the desired structure for antigen binding. For a review of scFv, see Plückthun, 1994, In: The Pharmacology of Monoclonal Antibodies, Vol. 113, Rosenburg and Moore eds. Springer-Verlag, New York, pp. 269-315.

The term “diabodies” refers to small antibody fragments with two antigen-binding sites, in which fragments comprise a heavy chain variable domain (VH) connected to a light chain variable domain (VL) in the same polypeptide chain (VH and VL).By using a linker that is too short to allow pairing between the two variable domains on the same chain, the variable domains are forced to pair with complementary domains of another chain, creating two antigen-binding sites. Diabodies are described more fully, for example, in EP 0404097; WO 93/11161; and Hollinger et al. Proc. Natl. Acad. Sci. USA (1993) 90:6444-6448.

The term “linear antibodies” refers to antibodies as described in Zapata et al. Protein Eng. (1995) 8(10):1057-1062. Briefly, these antibodies contain a pair of tandem Fd segments (VH-CH1-VH-CH1) that, together with complementary light chain polypeptides, form a pair of antigen binding regions. Linear antibodies can be bispecific or monospecific.

The term “monobody” as used herein, refers to an antigen binding molecule with a heavy chain variable domain and no light chain variable domain. A monobody can bind to an antigen in the absence of light chains and typically has three hypervariable regions, for example CDRs designated CDRH1, CDRH2, and CDRH3. A heavy chain IgG monobody has two heavy chain antigen binding molecules connected by a disulfide bond. The heavy chain variable domain comprises one or more hypervariable regions, preferably a CDRH3 or HVL-H3 region.

Antibody fragments may be obtained using conventional recombinant or protein engineering techniques and the fragments can be screened for binding to Protein S, or another function, in the same manner as intact antibodies.

Antibody fragments of the invention may be made by truncation, e.g. by removal of one or more amino acids from the N and/or C-terminal ends of a polypeptide. Fragments may also be generated by one or more internal deletions.

An antibody of the invention may be, or may comprise, a fragment of the anti-Protein S antibody or a variant hereof.

An antibody of the invention may be, or may comprise, an antigen binding portion of one of these antibodies, or variants thereof. For example, the antibody of the invention may be a Fab fragment of one of these antibodies or variants thereof, or it may be a single chain antibody derived from one of these antibodies, or a variant thereof.

A variant antibody may comprise 1, 2, 3, 4, 5, up to 10, up to 20, up to 30 or more amino acid substitutions and/or deletions and/or insertions from the specific sequences and fragments discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Insertion” variants may comprise the insertion of individual amino acids, insertion of small groups of amino acids such as 2, 3, 4 or 5 amino acids, or insertion of larger amino acid regions, such as the insertion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | |
|---|---|
| Ala (A) | aliphatic, hydrophobia, neutral |
| Cys (C) | polar, hydrophobic, neutral |
| Asp (D) | polar, hydrophilic, charged (−) |
| Glu (E) | polar, hydrophilic, charged (−) |
| Phe (F) | aromatic, hydrophobic, neutral |
| Gly (G) | aliphatic, neutral |
| His (H) | aromatic, polar, hydrophilic, charged (+) |
| Ile (I) | aliphatic, hydrophobic, neutral |
| Lys (K) | polar, hydrophilic, charged(+) |
| Leu (L) | aliphatic, hydrophobic, neutral |
| Met (M) | hydrophobic, neutral |
| Asn (N) | polar, hydrophilic, neutral |
| Pro (P) | hydrophobic, neutral |
| Gln (Q) | polar, hydrophilic, neutral |
| Arg (R) | polar, hydrophilic, charged (+) |
| Ser (S) | polar, hydrophilic, neutral |
| Thr (T) | polar, hydrophilic, neutral |
| Val (V) | aliphatic, hydrophobic, neutral |
| Trp (W) | aromatic, hydrophobic, neutral |
| Tyr (Y) | aromatic, polar, hydrophobic |

Preferred “derivatives” or “variants” include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected.

Substitutions may be, but are not limited to, conservative substitutions.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

The term “human antibody”, as used herein, is intended to include antibodies having variable regions in which at least a portion of a framework region and/or at least a portion of a CDR region are derived from human germline immunoglobulin sequences. For example, a human antibody may have variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo).

Such a human antibody may be a human monoclonal antibody. Such a human monoclonal antibody may be produced by a hybridoma which includes a B cell obtained from a transgenic nonhuman animal, e.g., a transgenic mouse, having a genome comprising human immunoglobulin heavy and light chain gene segments repertoires, fused to an immortalized cell.

Human antibodies may be isolated from sequence libraries built on selections of human germline sequences, further diversified with natural and synthetic sequence diversity.

Human antibodies may be prepared by in vitro immunisation of human lymphocytes followed by transformation of the lymphocytes with Epstein-Barr virus.

The term “human antibody derivative” refers to any modified form of the human antibody, such as a conjugate of the antibody and another agent or antibody.

The term “humanised antibody”, as used herein, refers to a human/non-human chimeric antibody that contains a sequence (CDR regions or parts thereof) derived from a non-human immunoglobulin. A humanised antibody is, thus, a human immunoglobulin (recipient antibody) in which at least residues from a hyper-variable region of the recipient are replaced by residues from a hyper-variable region of an antibody from a non-human species (donor antibody) such as from a mouse, rat, rabbit or non-human primate, which have the desired specificity, affinity, sequence composition and functionality. In some instances, FR residues of the human immunoglobulin are replaced by corresponding non-human residues. An example of such a modification is the introduction of one or more so-called back-mutations, which are typically amino acid residues derived from the donor antibody. Humanisation of an antibody may be carried out using recombinant techniques known to the person skilled in the art (see, e.g., Antibody Engineering, Methods in Molecular Biology, vol. 248, edited by Benny K. Lo). A suitable human recipient framework for both the light and heavy chain variable domain may be identified by, for example, sequence or structural homology. Alternatively, fixed recipient frameworks may be used, e.g., based on knowledge of structure, biophysical and biochemical properties. The recipient frameworks can be germline derived or derived from a mature antibody sequence. CDR regions from the donor antibody can be transferred by CDR grafting.

The CDR grafted humanised antibody can be further optimised for e.g. affinity, functionality and biophysical properties by identification of critical framework positions where re-introduction (backmutation) of the amino acid residue from the donor antibody has beneficial impact on the properties of the humanised antibody. In addition to donor antibody derived backmutations, the humanised antibody can be engineered by introduction of germline residues in the CDR or framework regions, elimination of immunogenic epitopes, site-directed mutagenesis, affinity maturation, etc.

Furthermore, humanised antibodies may comprise residues that are not found in the recipient antibody or in the donor antibody. These modifications are made to further refine antibody performance. In general, a humanised antibody will comprise at least one—typically two—variable domains, in which all or substantially all of the CDR regions correspond to those of a non-human immunoglobulin and in which all or substantially all of the FR residues are those of a human immunoglobulin sequence. The humanised antibody can, optionally, also comprise at least a portion of an immunoglobulin constant region (Fc), typically that of a human immunoglobulin.

The term “humanised antibody derivative” refers to any modified form of the humanised antibody, such as a conjugate of the antibody and another agent or antibody.

The term “chimeric antibody”, as used herein, refers to an antibody whose light and heavy chain genes have been constructed, typically by genetic engineering, from immunoglobulin variable and constant region genes that originate from different species. For example, the variable segments of genes from a mouse monoclonal antibody may be joined to human constant regions.

The fragment crystallizable region (“Fc region”/“Fc domain”) of an antibody is the N-terminal region of an antibody, which comprises the constant CH2 and CH3 domains. The Fc domain may interact with cell surface receptors called Fc receptors, as well as some proteins of the complement system. The Fc region enables antibodies to interact with the immune system. In one aspect of the invention, antibodies may be engineered to include modifications within the Fc region, typically to alter one or more of its functional properties, such as serum half-life, complement fixation, Fc-receptor binding, protein stability and/or antigen-dependent cellular cytotoxicity, or lack thereof, among others. Furthermore, an antibody of the invention may be chemically modified (e.g., one or more chemical moieties can be attached to the antibody) or be modified to alter its glycosylation, again to alter one or more functional properties of the antibody. An IgG1 antibody may carry a modified Fc domain comprising one or more, and perhaps all of the following mutations that will result in decreased affinity to certain Fc receptors (L234A, L235E, and G237A) and in reduced C1q-mediated complement fixation (A330S and P331S), respectively (residue numbering according to the EU index).

The isotype of an antibody of the invention may be IgG, such as IgG1, such as IgG2, such as IgG4. If desired, the class of an antibody may be “switched” by known techniques. For example, an antibody that was originally produced as an IgM molecule may be class switched to an IgG antibody. Class switching techniques also may be used to convert one IgG subclass to another, for example: from IgG1 to IgG2 or IgG4; from IgG2 to IgG1 or IgG4; or from IgG4 to IgG1 or IgG2. Engineering of antibodies to generate constant region chimeric molecules, by combination of regions from different IgG subclasses, can also be performed.

In one embodiment, the hinge region of CH1 is modified such that the number of cysteine residues in the hinge region is altered, e.g., increased or decreased. This approach is described further for instance in U.S. Pat. No. 5,677,425 by Bodmer et al.

The constant region may be modified to stabilize the antibody, e.g., to reduce the risk of a bivalent antibody separating into two monovalent VH-VL fragments. For example, in an IgG4 constant region, residue S228 (according to the EU index numbering index, S241 according to Kabat) may be mutated to a proline (P) residue to stabilise inter heavy chain disulphide bridge formation at the hinge (see, e.g., Angal et al. Mol Immunol. (1993) 30:105-8).

Antibodies or fragments thereof may be defined in terms of their complementarity-determining regions (CDRs). The term “complementarity-determining region” or “hypervariable region”, when used herein, refers to the regions of an antibody in which amino acid residues involved in antigen binding are situated. The region of hypervariability or CDRs can be identified as the regions with the highest variability in amino acid alignments of antibody variable domains. Databases can be used for CDR identification such as the Kabat database, the CDRs e.g. being defined as comprising amino acid residues 24-34 (L1), 50-56 (L2) and 89-97 (L3) of the light-chain variable domain and 31-35 (H1), 50-65 (H2) and 95-102 (H3) in the heavy-chain variable domain; (Kabat et al. (1991) Sequences of Proteins of Immunological Interest, Fifth Edition, U.S. Department of Health and Human Services, NIH Publication No. 91-3242) Alternatively CDRs can be defined as those residues from a “hypervariable loop” (residues 26-33 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol (1987) 196:901-917). Typically, the numbering of amino acid residues in this region is performed by the method described in Kabat et al. supra. Phrases such as “Kabat position”, “Kabat residue”, and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a framework (FR) or CDR of the variable domain. For example, a heavy chain variable domain may include amino acid insertions (residue 52a, 52b and 52c according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “framework region” or “FR” residues refer to those VH or VL amino acid residues that are not within the CDRs, as defined herein.

An antibody of the invention may comprise a CDR region from one or more of the specific antibodies disclosed herein.

The term “epitope”, as used herein, is defined in the context of a molecular interaction between an “antigen binding polypeptide” (Ab) and its corresponding “antigen” (Ag).

As used herein, the term “Ab” include, but is not limited to, antibodies, a Fab, F(ab′)₂ or a Fv fragment, that specifically binds the corresponding Ag. The term “Ag” refers to the molecular entity used for immunization of an immunocompetent vertebrate to produce the Ab that recognizes the Ag. Herein, Ag is termed more broadly and is generally intended to include molecules that are specifically recognized by the Ab, thus including fragments or mimics of the molecule used in the immunization process for raising the Ab.

Generally, “epitope” refers to the area or region on an Ag to which an Ab specifically binds, i.e., the area or region in physical contact with the Ab. Physical contact may be defined using various criteria (e.g. a distance cut-off of 2-6 Å, such as 3 Å, such as 4 Å, such as 5 Å; or solvent accessibility) for atoms in the Ab and Ag molecules. A protein epitope may comprise amino acid residues in the Ag that are directly involved in binding to a Ab (also called the immunodominant component of the epitope) and other amino acid residues, which are not directly involved in binding, such as amino acid residues of the Ag which are effectively blocked by the Ab, i.e., amino acid residues within the “solvent-excluded surface” and/or the “footprint” of the Ab.

At its most detailed level, the epitope for the interaction between the Ag and the Ab can be described by the spatial coordinates defining the atomic contacts present in the Ag-Ab interaction, as well as information about their relative contributions to the binding thermodynamics.

At a less detailed level, the epitope can be characterized by the spatial coordinates defining the atomic contacts between the Ag and Ab.

At an even less detailed level the epitope can be characterized by the amino acid residues that it comprises as defined by a specific criteria such as the distance between or solvent accessibility of atoms in the Ab:Ag complex.

At a further less detailed level the epitope can be characterized through function, e.g. by competition binding with other Abs. The epitope can also be defined more generically as comprising amino acid residues for which substitution by another amino acid will alter the characteristics of the interaction between the Ab and Ag.

The epitope for a given Ab/Ag pair can be defined and characterized at different levels of detail using a variety of experimental and computational epitope mapping methods. The experimental methods include mutagenesis, X-ray crystallography, Nuclear Magnetic

Resonance (NMR) spectroscopy, Hydrogen deuterium eXchange Mass Spectrometry (HX-MS) and various competition binding methods. As each method relies on a unique principle the description of an epitope is intimately linked to the method by which it has been determined. Thus, the epitope for a given Ab/Ag pair will be defined differently depending on the epitope mapping method employed.

From the fact that descriptions and definitions of epitopes, dependent on the epitope mapping method used, are obtained at different levels of detail, it follows that comparison of epitopes for different Ab on the same Ag can similarly be conducted at different levels of detail.

Epitopes described on the amino acid level, e.g. determined from an X-ray structure, are said to be identical if they contain the same set of amino acid residues. Epitopes are said to overlap if at least one amino acid is shared by the epitopes. Epitopes are said to be separate (unique) if no amino acid residue is shared by the epitopes.

Epitopes characterized by competition binding are said to be overlapping if the binding of the corresponding Ab's are mutually exclusive, i.e., binding of one Ab excludes simultaneous binding of the other Ab. The epitopes are said to be separate (unique) if the Ag is able to accommodate binding of both corresponding Ab's simultaneously.

Generally, the term “paratope” refers to the area or region on the Ab to which an Ag specifically binds.

The term epitope herein includes both types of binding sites in any particular region of Protein S that specifically binds to an anti-Protein S antibody, or another Protein S-specific agent according to the invention, unless otherwise stated (e.g., in some contexts the invention relates to antibodies that bind directly to particular amino acid residues). Protein S may comprise a number of different epitopes, which may include, without limitation, (1) linear peptide antigenic determinants, (2) conformational antigenic determinants which consist of one or more non-contiguous amino acids located near each other in the mature Protein S conformation; and (3) post-translational antigenic determinants which consist, either in whole or part, of molecular structures covalently attached to Protein S, such as carbohydrate groups.

The definition of the term “paratope” is derived from the above definition of “epitope” by reversing the perspective. Thus, the term “paratope” refers to the area or region on the Ab to which an Ag specifically binds, i.e., with which it makes physical contact to the Ag.

The epitope and paratope for a given antibody/antigen pair may be identified by routine methods. For example, the general location of an epitope may be determined by assessing the ability of an antibody to bind to different Protein S fragments. The specific amino acids within Protein S that make contact with an antibody (epitope) and the specific amino acids in an antibody that make contact with Protein S (paratope) may also be determined using routine methods. For example, the antibody and target molecule may be combined and the Ab:Ag complex may be crystallised. The crystal structure of the complex may be determined and used to identify specific sites of interaction between the antibody and its target.

Antibodies that bind to the same antigen can be characterised with respect to their ability to bind to their common antigen simultaneously and may be subjected to “competition binding”/“binning”. In the present context, the term “binning” refers to a method of grouping antibodies that bind to the same antigen. “Binning” of antibodies may be based on competition binding of two antibodies to their common antigen in assays based on standard techniques such as surface plasmon resonance (SPR), Biolayer Interferometry, ELISA or flow cytometry.

An antibody's “bin” can be defined using a single reference antibody or, alternatively, a group of reference antibodies. The resolution on the “bin” identification for a given antibody will increase with the number of reference antibodies used. When using a single reference antibody, if a second antibody is unable to bind to an antigen at the same time as the reference antibody, the second antibody is said to belong to the same “bin” as the reference antibody. In this case, the reference and the second antibody competitively bind to the same part of the antigen and are coined “competing antibodies”. If a second antibody is capable of binding to an antigen at the same time as the reference antibody, the second antibody is said to belong to a separate “bin”. In this case, the reference and the second antibody do not competitively bind to the same part of the antigen and are coined “non-competing antibodies”. When using a group of reference antibodies for “bin” identification, said group of reference antibodies can comprise a group of known or novel antibodies which can be used to define individual antibody “bins” by cross competition analyses, where each antibody within the group is assayed for competition for antigen binding with each member of the group. Antibody A is said to belong to the same “bin” as antibody B when they exhibit the same pattern of binding in the cross-competition analyses. Antibody A is said to belong to a different “bin” than antibody B, when they exhibit a different competition binding profile against one or more of the individual antibodies in the reference group. The competition binding profile is the compiled set of data where each antibody within the group is assayed for the ability to bind antigen at the same time as another member of the group. E.g. the antigen binding profile for antibody A relative to a reference group of antibody 1, 2 and 3 is as follows: A+1=no binding by A; A+2=binding by A; A+3=binding by A. Antibody B has a different competition binding profile compared to antibody A and the two antibodies are said to belong to different “bins” if: B+1=binding by B; B+2=binding by B; B+3=binding by B. Antibody C has a similar binding profile compared to antibody A and the two antibodies are said to belong to the same “bin” if: C+1=no binding by C; C+2=binding by C; C+3=binding by C. As stated the resolution on the “bin” identification for a given antibody will increase with the number of reference antibodies used. Competitive binding assays do not provide information on binding affinities and the assay must be designed in such a way that the tested antibodies are individually capable of binding the antigen sufficiently enough to function as binding competitors.

Antibody “binning” does not provide direct information about the epitope. Competing antibodies, i.e., antibodies belonging to the same “bin” may have identical epitopes, overlapping epitopes or even separate epitopes. The latter is the case if the reference antibody bound to its epitope on the antigen takes up the space required for the second antibody to contact its epitope on the antigen (“steric hindrance”). Non-competing antibodies generally have separate epitopes.

The term “binding affinity” is herein used as a measure of the strength of a non-covalent interaction between two molecules, e.g. an antibody, or fragment thereof, and an antigen. The term “binding affinity” is used to describe monovalent interactions (intrinsic activity).

Binding affinity between two molecules, e.g. an antibody, or fragment thereof, and an antigen, through a monovalent interaction may be quantified by determining the equilibrium dissociation constant (K_{D}). In turn, K_{D} can be determined by measurement of the kinetics of complex formation and dissociation, e.g. by the SPR method. The rate constants corresponding to the association and the dissociation of a monovalent complex are referred to as the association rate constant kₐ (or kₒₙ) and dissociation rate constant k_{d} (or k_{off}), respectively. K_{D} is related to kₐ and k_{d} through the equation K_{D}=k_{d}/kₐ.

Following the above definition, binding affinities associated with different molecular interactions, such as comparison of the binding affinity of different antibodies for a given antigen, may be compared by comparison of the K_{D} values for the individual antibody/antigen complexes.

An antibody according to the current invention may be able to compete with another molecule, such as a naturally occurring ligand or receptor or another antibody, for binding to Protein S. Therefore, an antibody according to the current invention may be able to bind Protein S with a greater affinity that that of another molecule also capable of binding Protein S.

The ability of an antibody to compete with a natural ligand/receptor for binding to an antigen may be assessed by determining and comparing the K_{D} value for the interactions of interest, such as a specific interaction between an antibody and an antigen, with that of the K_{D} value of an interaction not of interest. Typically, the K_{D} for the antibody with respect to the target will be at least 5-fold, more preferably 10-fold less than K_{D} with respect to the other, non-target molecule such as unrelated material or accompanying material in the environment. More preferably, the K_{D} will be at least 50-fold less, such as 100-fold less, or 200-fold less; even more preferably at least 500-fold less, such as 1,000-fold less, or 10,000-fold less.

The value of this dissociation constant can be determined directly by well-known methods. Standard assays to evaluate the binding ability of ligands such as antibodies towards targets are known in the art and include, for example, ELISAs, Western blots, RIAs, and flow cytometry analysis. The binding kinetics and binding affinity of the antibody also can be assessed by standard assays known in the art, such as SPR.

A competitive binding assay can be conducted in which the binding of the antibody to the target is compared to the binding of the target by another ligand of that target, such as another antibody.

### Polynucleotides

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences.

The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

In one embodiment, a polynucleotide of the invention comprises a sequence which encodes a VH or VL amino acid sequence as described above. For example, a polynucleotide of the invention may encode a polypeptide comprising the sequence of SEQ ID NOs: 49-55, or a variant or fragment thereof as described above. A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5 up to 10, up to 20, up to 30, up to 40, up to 50, up to 75 or more nucleic acid substitutions and/or deletions from the sequences given in the sequence listing.

An antibody of the invention may thus be produced from or delivered in the form of a polynucleotide which encodes, and is capable of expressing, it. Where the antibody comprises two or more chains, a polynucleotide of the invention may encode one or more antibody chains. For example, a polynucleotide of the invention may encode an antibody light chain, an antibody heavy chain or both. Two polynucleotides may be provided, one of which encodes an antibody light chain and the other of which encodes the corresponding antibody heavy chain. Such a polynucleotide or pair of polynucleotides may be expressed together such that an antibody of the invention is generated.

Polynucleotides of the invention can be synthesised according to methods well known in the art, as described by way of example in Sambrook et al. (1989, Molecular Cloning—a laboratory manual; Cold Spring Harbor Press).

The nucleic acid molecules of the present invention may be provided in the form of an expression cassette which includes control sequences, signal peptide sequences operably linked to the inserted sequence, thus allowing for expression of the antibody of the invention in vivo. These expression cassettes, in turn, are typically provided within vectors (e.g., plasmids or recombinant viral vectors). Such an expression cassette may be administered directly to a host subject.

Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers, signal peptide sequences and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

The invention also includes cells that have been modified to express an antibody of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for an antibody of the invention include mammalian HEK293, CHO, BHK, NSO and human retina cells. Preferably the cell line selected will be one which is not only stable, but also allows for mature glycosylation and cell surface expression of a polypeptide.

Such cell lines of the invention may be cultured using routine methods to produce an antibody of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

### Pharmaceutical Compositions

In another aspect, the present invention provides compositions and formulations comprising molecules of the invention, such as the antibodies, polynucleotides, vectors and cells as described herein. For example, the invention provides a pharmaceutical composition that comprises one or more antibodies of the invention, formulated together with a pharmaceutically acceptable carrier.

Accordingly, one object of the invention is to provide a pharmaceutical composition comprising such an antibody which is present in a concentration from 0.25 mg/ml to 250 mg/ml, and wherein said composition has a pH from 2.0 to 10.0. The composition may further comprise one or more of a buffer system, a preservative, a tonicity agent, a chelating agent, a stabilizer, or a surfactant, as well as various combinations thereof. In some embodiments, at least one of the preservatives, isotonic agents, chelating agents, stabilizers and surfactants can be included in pharmaceutical compositions described herein. Reference may be made to Remington: The Science and Practice of Pharmacy, 19th edition, 1995.

In one embodiment, the pharmaceutical composition is an aqueous formulation. Such a formulation is typically a solution or a suspension, but may also include colloids, dispersions, emulsions, and multi-phase materials. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. In another embodiment, the pharmaceutical composition is a freeze-dried formulation, to which the physician or the patient adds solvents and/or diluents prior to use.

In a further aspect, the pharmaceutical composition comprises an aqueous solution of such an antibody, and a buffer, wherein the antibody is present in a concentration from 1 mg/ml or above, and wherein said formulation has a pH from about 2.0 to about 10.0.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Depending on the route of administration, the antibody may be coated in a material to protect the antibody from the action of acids and other natural conditions that may inactivate or denature the antibody.

Preferred pharmaceutically acceptable carriers comprise aqueous carriers or diluents. Examples of suitable aqueous carriers that may be employed in the pharmaceutical compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

A pharmaceutical composition of the invention also may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. antibody) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Pharmaceutical compositions of the invention may comprise additional active ingredients as well as an antibody of the invention. As mentioned above, compositions of the invention may comprise one or more antibodies of the invention. They may also comprise additional therapeutic or prophylactic agents. For example, where a pharmaceutical composition of the invention is intended for use in the treatment of a bleeding disorder, it may additionally comprise one or more agents intended to reduce the symptoms of the bleeding disorder. For example, the composition may comprise one or more clotting factors. The composition may comprise one or more other components intended to improve the condition of the patient. For example, where the composition is intended for use in the treatment of patients suffering from unwanted bleeding such as patients undergoing surgery or patients suffering from trauma, the composition may comprise one or more analgesic, anaesthetic, immunosuppressant or anti-inflammatory agents. Also falling within the scope of the present invention are kits comprising antibodies or other compositions of the invention and instructions for use. Such a kit may further contain one or more additional reagents, such as an additional therapeutic or prophylactic agent as discussed above.

### Mode of Administration

An antibody or antigen-binding fragment thereof or pharmaceutical composition of the invention may be administered via one or more routes of administration using one or more of a variety of methods known in the art. As will be appreciated by the skilled artisan, the route and/or mode of administration will vary depending upon the desired results.

Preferred routes of administration for antibodies or compositions of the invention include intravenous, intramuscular, intradermal, intraperitoneal, subcutaneous, spinal or other parenteral routes of administration, for example by injection or infusion.

Alternatively, an antibody of the invention may be administered via a non-parenteral route, such as perorally or topically.

An antibody of the invention may be administered prophylactically or therapeutically (on demand).

The phrase “parenteral administration” as used herein means modes of administration other than enteral and topical administration, usually by injection. Alternatively, an antibody or composition of the invention can be administered via a non-parenteral route, such as a topical, epidermal or mucosal route of administration.

Similarly, an antibody of the invention may be used for the manufacture of a medicament suitable for parenteral administration.

An antibody of the invention may be used for the manufacture of a medicament suitable for intravenous administration.

An antibody of the invention may be used for the manufacture of a medicament suitable for intramuscular administration.

An antibody of the invention may be used for the manufacture of a medicament suitable for subcutaneous administration.

### Dosages

A suitable dosage of an antibody of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular antibody employed, the route of administration, the time of administration, the rate of excretion of the antibody, the duration of the treatment, other drugs, compounds and/or materials used in combination with the particular compositions employed, the age, sex, weight, condition, general health and prior medical history of the patient being treated, and like factors well known in the medical arts.

A suitable dose of an antibody of the invention may be, for example, in the range of from about 0.1 μg/kg to about 100 mg/kg body weight of the patient to be treated. For example, a suitable dosage may be from about 1 μg/kg to about 10 mg/kg body weight per day or from about 1 mg/kg to about 5 mg/kg body weight per day. A suitable dose of an antibody of the invention may be in the range of from 2 to 200 mg/kg, such as about 150-200 mg/kg, such as about 150-170 mg/kg, such as about 100-150 mg/kg, such as about 50-100 mg/kg, such as about 70-90 mg/kg, such as about 10-50 mg/kg, such as about 10-30 mg/kg. Other suitable dosages may be approximately 0.1-10 mg/kg, such as approximately 0.1-1 mg/kg, such as approximately 1-2 mg/kg or approximately 2-3 mg/kg or approximately 4-5 mg/kg or approximately 5-6 mg/kg or approximately 6-7 mg/kg or approximately 7-8 mg/kg or approximately 8-9 mg/kg or approximately 9-10 mg/kg; or approximately 10-21 mg/kg, such as approximately 10-11 mg/kg, or approximately 11-12 mg/kg, or approximately 12-13 mg/kg, or approximately 13-14 mg/kg, or approximately 14-15 mg/kg, or approximately 15-16 mg/kg, or approximately 16-17 mg/kg, or approximately 17-18 mg/kg, or approximately 18-19 mg/kg, or approximately 19-20 mg/kg or approximately 20-21 mg/kg. The amount of monoclonal antibody administered to a subject may be such that its administration results in a subject plasma concentration of about 10 μg/ml to about 40 μg/ml, such as about 15-35 μg/ml, such as about 10-15 μg/ml, such as about 15-20 μg/ml, such as about 20-25 μg/ml, such as about 25-30 μg/ml, such as about 30-35 μg/ml, such as about 35-40 μg/ml, of said monoclonal antibody.

### Dosage Regimens

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Antibodies may be administered in a single dose or in multiple doses. The multiple doses may be administered via the same or different routes and to the same or different locations. Alternatively, antibodies can be administered as a sustained release formulation, in which case less frequent administration is required. Dosage and frequency may vary depending on the half-life of the antibody in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Thus, an antibody of the invention may be administered: approximately daily, approximately every other day, approximately every third day, approximately every fourth day, approximately every fifth day, approximately every sixth day; approximately every week, such as every 5, 6, 7, 8, 9 or 10 days; approximately every other week, such as every 11, 12, 13, 14, 15, 16 or 17 days; approximately every third week, such as every 18, 19, 20, 21, 22, 23 or 24 days; approximately every fourth week, such as every 25, 26, 27, 28, 29, 30 or 31 days.

An antibody of the invention may also be administered on-demand.

### Further Embodiments

The follow embodiments are provided to aid understanding of the present invention, however, the present invention is not limited only to the follow the below embodiments.

In one embodiment the invention relates to inhibitors (such as but not limited to antibodies, Fabs or other fragments, peptides or aptamers) that bind to Protein S and inhibit Protein S interaction with APC.

In one embodiment the invention relates to antibodies or antigen-binding fragments thereof that bind to Protein S and inhibit Protein S interaction with APC.

In one embodiment the invention relates to antibodies or antigen-binding fragments thereof that bind to Protein S and inhibit Protein S interaction with APC without interfering with known non-coagulant functions of Protein S.

In one embodiment the invention relates to the use of antibodies or antigen-binding fragment thereof that bind to Protein S and inhibit Protein S interaction with APC in the treatment of a coagulopathy, such as haemophilia.

In one embodiment the invention relates to the use of antibodies or antigen-binding fragment thereof that bind to Protein S and prevent the interaction with APC without interfering with known non-coagulant functions of Protein S for haemophilia treatment.

In one embodiment the invention relates to the use of inhibitors that bind to Protein S in the treatment of a coagulopathy, such as haemophilia independently of APC.

In one embodiment the invention relates to the use of inhibitors that bind to Protein S without interfering with known non-coagulant functions of Protein S for haemophilia treatment independently of APC.

In one embodiment the invention relates to the use of antibodies or antigen-binding fragment thereof that bind to Protein S in the treatment of a coagulopathy, such as haemophilia independently of APC.

In one embodiment the invention relates to the use of antibodies or antigen-binding fragment thereof that bind to Protein S without interfering with known non-coagulant functions of Protein S for haemophilia treatment independently of APC.

In one embodiment the present invention provides a method of treatment of a coagulopathy using a Protein S inhibitor capable of binding in the EGF1-4 region of Protein S.

In one embodiment the present invention provides a method for treatment of a coagulopathy using a Protein S inhibitor capable of binding in the EGF1-3 region of Protein S.

In one embodiment the present invention provides a method for treatment of a coagulopathy using a Protein S inhibitor capable of binding in the EGF1-2 region of Protein S.

In one embodiment the present invention provides a method for treatment of a coagulopathy using a Protein S inhibitor capable of binding in the EGF1 region of Protein S.

In one embodiment the present invention provides a method of treatment of a coagulopathy using an anti-Protein S antibody or antigen binding fragment thereof capable of binding in the EGF1-4 region of Protein S.

In one embodiment the present invention provides a method of treatment of a coagulopathy using an anti-Protein S antibody or antigen binding fragment thereof capable of binding in the EGF1-3 region of Protein S.

In one embodiment the present invention provides a method of treatment of a coagulopathy using an anti-Protein S antibody or antigen binding fragment thereof capable of binding in the EGF1-2 region of Protein S.

In one embodiment the present invention provides a method of treatment of a coagulopathy using an anti-Protein S antibody or antigen binding fragment thereof capable of binding in the EGF1 region of Protein S.

In one embodiment the present invention provides the use of a Protein S inhibitor capable of binding in the EGF1-4 region of Protein S for the manufacture of a medicament for use in the treatment of a coagulopathy.

In one embodiment the present invention provides the use of a Protein S inhibitor capable of binding in the EGF1-3 region of Protein S for the manufacture of a medicament for use in the treatment of a coagulopathy.

In one embodiment the present invention provides the use of a Protein S inhibitor capable of binding in the EGF1-2 region of Protein S for the manufacture of a medicament for use in the treatment of a coagulopathy.

In one embodiment the present invention provides the use of a Protein S inhibitor capable of binding in the EGF1 region of Protein S for the manufacture of a medicament for use in the treatment of a coagulopathy.

In one embodiment the present invention provides the use of an anti-Protein S antibody or antigen-binding fragment thereof capable of binding in the EGF1-4 region of Protein S for the manufacture of a medicament for use in the treatment of a coagulopathy.

In one embodiment the present invention provides the use of an anti-Protein S antibody or antigen-binding fragment thereof capable of binding in the EGF1-3 region of Protein S for the manufacture of a medicament for use in the treatment of a coagulopathy.

In one embodiment the present invention provides the use of an anti-Protein S antibody or antigen-binding fragment thereof capable of binding in the EGF1-2 region of Protein S for the manufacture of a medicament for use in the treatment of a coagulopathy.

In one embodiment the present invention provides the use of an anti-Protein S antibody or antigen-binding fragment thereof capable of binding in the EGF1 region of Protein S for the manufacture of a medicament for use in the treatment of a coagulopathy.

In one embodiment said coagulopathy is haemophilia, such as haemophilia A or B.

In one embodiment an antibody or antigen-binding fragment thereof of the present invention binds to human Protein S.

In one embodiment an antibody or antigen-binding fragment thereof of the present invention binds to Protein S from Macaca fascicularis.

In one embodiment an antibody or antigen-binding fragment thereof of the present invention binds to rabbit Protein S.

In one embodiment an antibody or antigen-binding fragment thereof of the present invention is capable of significantly reducing blood loss in vivo in a rabbit haemophilia model.

In one embodiment the present invention provides an antibody or antigen-binding fragment thereof which is capable of increasing thrombin generation in a human FVIII deficient plasma-based thrombin generation assay.

In one embodiment an antibody or antigen-binding fragment thereof of the present invention does not prevent binding of human Protein S to a lipid surface.

In one embodiment an antibody or antigen-binding fragment thereof of the present invention does not prevent binding of human Protein S to C4BP.

In one embodiment an antibody or antigen-binding fragment thereof of the present invention may be capable of binding its epitope in a Ca²⁺ independent manner.

In one embodiment an antibody or antigen-binding fragment thereof of the present invention has the ability to shorten clotting time in human FVIII-deficient plasma or to reduce time to clot as measured in a thromboelastography (TEG) analysis of human whole blood.

In one embodiment the antibody or antigen-binding fragment thereof of the present invention does not affect the cofactor function of Protein S on TFPI.

In one embodiment an antibody or antigen-binding fragment thereof of the present invention neither prevents binding of Protein S to a lipid surface nor C4BP nor TFPI whilst being cross-reactive against Protein S from different species while at the same time being useful in the treatment of a coagulopathy such as haemophilia.

In one embodiment an antibody of the invention may be a human antibody or a humanised antibody optionally comprising one or more back mutations.

In one embodiment the present invention provides antibodies or antigen-binding fragment thereof the half-life of which can be extended by applying known protraction principles including pegylation, acetylation etc.

In one embodiment an antibody or antigen-binding fragment thereof of the invention may comprise a CDR region from one or more of the specific antibodies disclosed herein, such as a CDR region from within any one of the variable light and variable heavy chain sequences represented by SEQ ID NOs: 4 to 45 and 49-55 as described herein (cf. also FIGS. 9 and 10 for annotated CDR sequences of SEQ ID NOs 4-45 and FIGS. 14 and 15 for annotated CDR sequences for SEQ ID NOs 49-55).

In one such embodiment the CDR sequences within the light chain of an antibody or antigen-binding fragment thereof of the invention are at residues SASSSVSYMY (CDR1 residues 24-33 of SEQ ID NO: 36), DTSNLAS (CDR2 residues 49-55 of SEQ ID NO: 36) and QQWSSYPLT (CDR3 residues 88-96 of SEQ ID NO: 36).

In one such embodiment the CDR sequences within the heavy chain of an antibody or antigen-binding fragment thereof of the invention are at residues TSGMGVS (CDR1 residues 31-37 of SEQ ID NO: 37), HIYWDDDKRYNPSLKS (CDR2 residues 52-67 of SEQ ID NO: 37) and YGNYGDY (CDR3 residues 100-106 of SEQ ID NO: 37).

In another such embodiment the CDR sequences within the light chain of an antibody or antigen-binding fragment thereof of the invention are at residues RASSSVSYMY (CDR1 residues 24-33 of SEQ ID NO: 40), ATSNLAS (CDR2 residues 49-55 of SEQ ID NO: 40) and QQWSSIPPT (CDR3 residues 88-96 of SEQ ID NO: 40).

In another such embodiment the CDR sequences within the heavy chain of an antibody or antigen-binding fragment thereof of the invention are at residues SYWIN (CDR1 residues 31-35 of SEQ ID NO: 41), RIDPYDSETHYNQKFKD (CDR2 residues 50-66 of SEQ ID NO: 41) and WGGSGYAMDY (CDR3 residues 99-108 of SEQ ID NO: 41).

In another such embodiment the CDR sequences within the light chain of an antibody or antigen-binding fragment thereof of the invention are at residues SVSSSVSYMH (CDR1 residues 24-33 of SEQ ID NO: 10), DTSNLVS (CDR2 residues 49-55 of SEQ ID NO: 10) and QQYSGYLYT (CDR3 residues 88-96 of SEQ ID NO: 10).

In another embodiment the CDR sequences within the heavy chain of an antibody or antigen-binding fragment thereof of the invention are at residues DAWMD (CDR1 residues 31-35 of SEQ ID NO: 11), EIRSKANNHATYYAESVKG (CDR2 residues 50-68 of SEQ ID NO: 11) and TTAFLFDY (CDR3 residues 101-108 of SEQ ID NO: 11).

In yet another such embodiment the CDR sequences within the light chain of an antibody or antigen-binding fragment thereof of the invention are at residues SATSSVTYMH (CDR1 residues 24-33 of SEQ ID NO: 26), STSNLAS (CDR2 residues 49-55 of SEQ ID NO: 26) and QQRSSYPPT (CDR3 residues 88-96 of SEQ ID NO: 26).

In another embodiment the CDR sequences within the heavy chain of an antibody or antigen-binding fragment thereof of the invention are at residues GYGVS (CDR1 residues 31-35 of SEQ ID NO: 27), MIWGDGTTDYNSTLKS (CDR2 residues 50-65 of SEQ ID NO: 27) and DPGAMDY (CDR3 residues 98-104 of SEQ ID NO: 27).

In yet another such embodiment the CDR sequences within the light chain of an antibody or antigen-binding fragment thereof of the invention are at residues SASSSVSYMY (CDR1 residues 24-33 of SEQ ID NO: 12), STSNLAS (CDR2 residues 49-55 of SEQ ID NO: 12) and QQWSSNPYT (CDR3 residues 88-96 of SEQ ID NO: 12).

In another embodiment the CDR sequences within the heavy chain of an antibody or antigen-binding fragment thereof of the invention are at residues SYWMN (CDR1 residues 31-35 of SEQ ID NO: 13), RIDPYDTETHYNQKFED (CDR2 residues 50-66 of SEQ ID NO: 13) and WAGSSYAMDY (CDR3 residues 99-108 of SEQ ID NO: 13).

In certain embodiments an antibody or antigen-binding fragment thereof of the invention may have one or more of the following CDR sequences within the light chain: RASSSVSYMY (CDR1 residues 24-33 of SEQ ID NO: 49), ATSNLAS (CDR2 residues 49-55 of SEQ ID NO: 49) and QQWSSIPPT (CDR3 residues 88-96 of SEQ ID NO: 49).

In certain embodiments an antibody or antigen-binding fragment thereof of the invention may have one or more of the following CDR sequences within the heavy chain: SYWIN (CDR1 residues 31-35 of SEQ ID NO: 50), RIDPYDSETHYAQKFQG (CDR2 residues 50-66 of SEQ ID NO: 50) and WGGSGYAMDY (CDR3 residues 99-108 of SEQ ID NO: 50).

In certain embodiments an antibody or antigen-binding fragment thereof of the invention may have one or more of the following CDR sequences within the light chain: RASSSVSYMY (CDR1 residues 24-33 of SEQ ID NO: 51), ATSNLAS (CDR2 residues 49-55 of SEQ ID NO: 51) and QQWSSIPPT (CDR3 residues 88-96 of SEQ ID NO: 51).

In certain embodiments an antibody or antigen-binding fragment thereof of the invention may have one or more of the following CDR sequences within the heavy chain: SYWIN (CDR1 residues 31-35 of SEQ ID NO: 52), RIDPYDSETHYAQKFQG (CDR2 residues 50-66 of SEQ ID NO: 52) and WGGSGYAMDY (CDR3 residues 99-108 of SEQ ID NO: 52).

In certain embodiments an antibody or antigen-binding fragment thereof of the invention may have one or more of the following CDR sequences within the light chain: RASSSVSYMY (CDR1 residues 24-33 of SEQ ID NO: 53), ATSNLAS (CDR2 residues 49-55 of SEQ ID NO: 53) and QQWSSIPPT (CDR3 residues 88-96 of SEQ ID NO: 53).

In certain embodiments an antibody or antigen-binding fragment thereof of the invention may have one or more of the following CDR sequences within the heavy chain: SYWIN (CDR1 residues 31-35 of SEQ ID NO: 54), RIDPYDSETHYAQKFQG (CDR2 residues 50-66 of SEQ ID NO: 54) and WGGSGYAMDY (CDR3 residues 99-108 of SEQ ID NO: 54).

In certain embodiments an antibody or antigen-binding fragment thereof of the invention may have one or more of the following CDR sequences within the heavy chain: SYWIN (CDR1 residues 31-35 of SEQ ID NO: 55), RIDPYDSETHYAQKFQG (CDR2 residues 50-66 of SEQ ID NO: 55) and WGGSGYAMDY (CDR3 residues 99-108 of SEQ ID NO: 55).

In certain embodiments an antibody or antigen-binding fragment thereof of the invention may have one or more of the following CDR sequences within the heavy chain: SYWIN (CDR1 residues 31-35 of SEQ ID NO: 52), RIDPYDSETHYNQKFKD (CDR2 residues 50-66 of SEQ ID NO: 41) and WGGSGYAMDY (CDR3 residues 99-108 of SEQ ID NO: 52).

In one embodiment potential aspartic acid sites in the CDR2 region which could potentially undergo isomerization to isoaspartic acid (isoAsp) in the antibody or antigen-binding fragment thereof are avoided by substituting amino acid residue D55 of SEQ ID NO:

50, 52, 54 or 55 with a different amino acid residue which is not cysteine (C).

In one embodiment the antibody or antigen-binding fragment thereof of the present invention comprises the following two CDR3 sequences (from light- and heavy chains, respectively):

QQYSGYLYT (CDR3 residues 88-96 of SEQ ID NO: 10) and

TTAFLFDY (CDR3 residues 101-108 of SEQ ID NO: 11).

In one embodiment the antibody or antigen-binding fragment thereof comprises the following two CDR3 sequences (from light- and heavy chains, respectively):

QQWSSNPYT (CDR3 residues 88-96 of SEQ ID NO: 12) and

WAGSSYAMDY (CDR3 residues 99-108 of SEQ ID NO: 13).

In one embodiment the antibody or antigen-binding fragment thereof comprises the following two CDR3 sequences (from light- and heavy chains, respectively):

QQRSSYPPT (CDR3 residues 88-96 of SEQ ID NO: 26) and

DPGAMDY (CDR3 residues 98-104 of SEQ ID NO: 27).

In one embodiment the antibody or antigen-binding fragment thereof comprises the following two CDR3 sequences (from light- and heavy chains, respectively):

QQWSSIPPT (CDR3 residues 88-96 of SEQ ID NO: 40) and

WGGSGYAMDY (CDR3 residues 99-108 of SEQ ID NO: 41).

In one embodiment the antibody or antigen-binding fragment thereof comprises the following two CDR3 sequences (from light- and heavy chains, respectively):

QQWSSIPPT (CDR3 residues 88-96 of SEQ ID NO: 49) and

WGGSGYAMDY (CDR3 residues 99-108 of SEQ ID NO: 50).

In one embodiment an antibody or antigen-binding fragment thereof of the present invention comprises the light chain variable region of SEQ ID NO: 10 and the heavy chain variable region of SEQ ID NO: 11.

In one embodiment an antibody or antigen-binding fragment thereof of the invention comprises the light chain variable region of SEQ ID NO: 12 and the heavy chain variable region of SEQ ID NO: 13.

In one embodiment an antibody or antigen-binding fragment thereof of the invention comprises the light chain variable region of SEQ ID NO: 26 and the heavy chain variable region of SEQ ID NO: 27.

In one embodiment an antibody or antigen-binding fragment thereof of the invention comprises the light chain variable region of SEQ ID NO: 40 and the heavy chain variable region of SEQ ID NO: 41.

In one embodiment an antibody or antigen-binding fragment thereof of the invention may comprise the light chain variable region of SEQ ID NO: 49 and the heavy chain variable region of SEQ ID NO: 50.

In certain embodiments an antibody or antigen-binding fragment thereof of the invention may comprise the light chain variable region of SEQ ID NO: 49,

wherein amino acid residue L45 is substituted with P, and optionally

L46 is substituted with W.

and

the heavy chain variable region of SEQ ID NO: 50, said heavy chain variable region optionally further comprising one or more of the substitutions selected from a group consisting of M70L, R72V, T74K and V79A.

In one such embodiment an antibody or antigen-binding fragment thereof of the invention may comprise the light chain variable region of SEQ ID NO: 51, and the heavy chain variable region of SEQ ID NO: 50.

In one such embodiment an antibody or antigen-binding fragment thereof of the invention may comprise the light chain variable region of SEQ ID NO: 51, and the heavy chain variable region of SEQ ID NO: 52.

In one such embodiment an antibody or antigen-binding fragment thereof of the invention may comprise the light chain variable region of SEQ ID NO: 51, and the heavy chain variable region of SEQ ID NO: 54.

In one such embodiment an antibody or antigen-binding fragment thereof of the invention may comprise the light chain variable region of SEQ ID NO: 51, and the heavy chain variable region of SEQ ID NO: 55.

In one such embodiment an antibody or antigen-binding fragment thereof of the invention may comprise the light chain variable region of SEQ ID NO: 53, and the heavy chain variable region of SEQ ID NO: 50.

In one such embodiment an antibody or antigen-binding fragment thereof of the invention may comprise the light chain variable region of SEQ ID NO: 53, and the heavy chain variable region of SEQ ID NO: 52.

In one such embodiment an antibody or antigen-binding fragment thereof of the invention may comprise the light chain variable region of SEQ ID NO: 53, and the heavy chain variable region of SEQ ID NO: 54.

In one such embodiment an antibody or antigen-binding fragment thereof of the invention may comprise the light chain variable region of SEQ ID NO: 53, and the heavy chain variable region of SEQ ID NO: 55.

In specific embodiments the following monoclonal antibodies or antigen-binding fragments thereof are comprised by the invention:

An antibody wherein the light chain of said antibody comprises SEQ ID NO: 56 and the heavy chain of said antibody comprises SEQ ID NO: 57.

An antibody wherein the light chain of said antibody comprises SEQ ID NO: 58 and the heavy chain of said antibody comprises SEQ ID NO: 57.

An antibody wherein the light chain of said antibody comprises SEQ ID NO: 58 and the heavy chain of said antibody comprises SEQ ID NO: 59.

An antibody wherein the light chain of said antibody comprises SEQ ID NO: 60 and the heavy chain of said antibody comprises SEQ ID NO: 57.

An antibody wherein the light chain of said antibody comprises SEQ ID NO: 58 and the heavy chain of said antibody comprises SEQ ID NO: 61.

An antibody wherein the light chain of said antibody comprises SEQ ID NO: 58 and the heavy chain of said antibody comprises SEQ ID NO: 62.

An antibody wherein the light chain of said antibody comprises SEQ ID NO: 60 and the heavy chain of said antibody comprises SEQ ID NO: 59.

An antibody wherein the light chain of said antibody comprises SEQ ID NO: 60 and the heavy chain of said antibody comprises SEQ ID NO: 61.

An antibody wherein the light chain of said antibody comprises SEQ ID NO: 60 and the heavy chain of said antibody comprises SEQ ID NO: 62.

In one embodiment an antibody or antigen-binding fragment thereof of the present invention is capable of binding to a human Protein S epitope which comprises one or more residues selected from the group consisting of

### C32, K33, P34, G35, W36, Q37, G38, E39, K40, C41, E42 and F43 of SEQ ID NO: 2.

In one embodiment an antibody or antigen-binding fragment thereof of the present invention is capable of binding to a human Protein S epitope which comprises one or more residues selected from the group consisting of

### C32, K33, P34, G35, W36, Q37, G38, E39, K40, C41 and E42 of SEQ ID NO: 2.

In one embodiment an antibody or antigen-binding fragment thereof of the present invention is capable of binding to a human Protein S epitope which comprises one or more residues selected from the group consisting of

### C32, K33, P34, G35, W36, Q37, G38, E39, K40 and C41 of SEQ ID NO: 2.

In one embodiment an antibody or antigen-binding fragment thereof of the present invention is capable of binding to a human Protein S epitope which comprises one or more residues selected from the group consisting of

C32, K33, P34, G35, W36, Q37, G38, E39 and K40 of SEQ ID NO: 2.

In one embodiment an antibody or antigen-binding fragment thereof of the present invention is capable of binding to a human Protein S epitope which comprises one or more residues selected from the group consisting of

C32, K33, P34, G35, W36, Q37, G38 and E39 of SEQ ID NO: 2.

In one embodiment an antibody or antigen-binding fragment thereof of the present invention is capable of binding to a human Protein S epitope which comprises one or more residues selected from the group consisting of

K33, P34, G35, W36, Q37, G38, E39, K40, C41, E42 and F43 of SEQ ID NO: 2.

In one embodiment an antibody or antigen-binding fragment thereof is capable of binding to a human Protein S epitope which comprises one or more residues selected from the group consisting of

P34, G35, W36, Q37, G38, E39, K40, C41, E42 and F43 of SEQ ID NO: 2.

In one embodiment an antibody or antigen-binding fragment thereof of the present invention is capable of binding to a human Protein S epitope which comprises one or more residues selected from the group consisting of

P34, G35, W36, Q37, G38, E39, K40, C41 and E42 of SEQ ID NO: 2.

In one embodiment an antibody or antigen-binding fragment thereof of the present invention is capable of binding to a human Protein S epitope which comprises one or more residues selected from the group consisting of

P34, G35, W36, Q37, G38, E39, K40 and C41 of SEQ ID NO: 2.

In one embodiment an antibody or antigen-binding fragment thereof of the present invention is capable of binding to a human Protein S epitope which comprises one or more residues selected from the group consisting of

G35, W36, Q37, G38, E39, K40, C41, E42 and F43 of SEQ ID NO: 2.

In one embodiment an antibody or antigen-binding fragment thereof of the present invention is capable of binding to a human Protein S epitope which comprises one or more residues selected from the group consisting of

G35, W36, Q37, G38, E39, K40, C41 and E42 of SEQ ID NO: 2.

In one embodiment an antibody or antigen-binding fragment thereof of the present invention is capable of binding to a human Protein S epitope which comprises one or more residues selected from the group consisting of G35, W36, Q37, G38, E39, K40 and C41 of SEQ ID NO: 2.

In one embodiment an antibody or antigen-binding fragment thereof of the present invention is capable of binding to a human Protein S epitope which comprises one or more residues selected from the group consisting of
S20, C21, K22, G24, C32, K33, P34, G35, W36, Q37, G38, E39, K40, C41, E42, F43 of SEQ ID NO: 2.

In one embodiment an antibody or antigen-binding fragment thereof of the present invention is capable of binding to a human Protein S epitope which comprises one or more residues selected from the group consisting of
S20, C21, K22, D23, G24, K25, A26, S27, F28, T29, C30, C32, K33, P34, G35, W36, Q37, G38, E39, K40, C41, E42, F43 of SEQ ID NO: 2.

In one embodiment an antibody or antigen-binding fragment thereof of the present invention is capable of binding an epitope comprising amino acid residue S20 of SEQ ID NO: 2.

In one embodiment an antibody or antigen-binding fragment thereof of the present invention is capable of binding an epitope comprising amino acid residue C21 of SEQ ID NO: 2.

In one embodiment an antibody or antigen-binding fragment thereof of the present invention is capable of binding an epitope comprising amino acid residue K22 of SEQ ID NO: 2.

In one embodiment an antibody or antigen-binding fragment thereof of the present invention is capable of binding an epitope comprising amino acid residue G24 of SEQ ID NO: 2.

In one embodiment an antibody or antigen-binding fragment thereof of the present invention is capable of binding an epitope comprising amino acid residue A26 of SEQ ID NO: 2.

In one embodiment an antibody or antigen-binding fragment thereof of the present invention is capable of binding an epitope comprising amino acid residue S27 of SEQ ID NO: 2.

In one embodiment an antibody or antigen-binding fragment thereof of the present invention is capable of binding an epitope comprising amino acid residue F28 of SEQ ID NO: 2.

In one embodiment an antibody or antigen-binding fragment thereof of the present invention is capable of binding an epitope comprising amino acid residue T29 of SEQ ID NO: 2.

In one embodiment an antibody or antigen-binding fragment thereof of the present invention is capable of binding an epitope comprising amino acid residue C30 of SEQ ID NO: 2.

In one embodiment an antibody or antigen-binding fragment thereof of the present invention is capable of binding an epitope comprising amino acid residue C32 of SEQ ID NO: 2.

In one embodiment an antibody or antigen-binding fragment thereof of the present invention is capable of binding an epitope comprising amino acid residue K33 of SEQ ID NO: 2.

In one embodiment an antibody or antigen-binding fragment thereof of the present invention is capable of binding an epitope comprising amino acid residue P34 of SEQ ID NO: 2.

In one embodiment an antibody or antigen-binding fragment thereof of the present invention is capable of binding an epitope comprising amino acid residue G35 of SEQ ID NO: 2.

In one embodiment an antibody or antigen-binding fragment thereof of the present invention is capable of binding an epitope comprising amino acid residue W36 of SEQ ID NO: 2.

In one embodiment an antibody or antigen-binding fragment thereof of the present invention is capable of binding an epitope comprising amino acid residue Q37 of SEQ ID NO: 2.

In one embodiment an antibody or antigen-binding fragment thereof of the present invention is capable of binding an epitope comprising amino acid residue G38 of SEQ ID NO: 2.

In one embodiment an antibody or antigen-binding fragment thereof of the present invention is capable of binding an epitope comprising amino acid residue E39 of SEQ ID NO: 2.

In one embodiment an antibody or antigen-binding fragment thereof of the present invention is capable of binding an epitope comprising amino acid residue K40 of SEQ ID NO: 2.

In one embodiment an antibody or antigen-binding fragment thereof of the present invention is capable of binding an epitope comprising amino acid residue C41 of SEQ ID NO: 2.

In one embodiment an antibody or antigen-binding fragment thereof of the present invention is capable of binding an epitope comprising amino acid residue E42 of SEQ ID NO: 2.

In one embodiment an antibody or antigen-binding fragment thereof of the present invention is capable of binding an epitope comprising amino acid residue F43 of SEQ ID NO: 2.

In one embodiment an antibody or antigen-binding fragment thereof of the present invention is capable of binding an epitope comprising amino acid residues W36, E39 and K40 of SEQ ID NO: 2.

In one embodiment an antibody or antigen-binding fragment thereof of the present invention is capable of binding an epitope comprising amino acid residues W36, E39 and K40 and one or more of C41, E42 and F43 of SEQ ID NO: 2.

In one embodiment an antibody or antigen-binding fragment thereof of the present invention is capable of binding an epitope comprising amino acid residues W36, E39, K40 and F43 and one or more of C41 and E42 of SEQ ID NO: 2.

In one embodiment an antibody or antigen-binding fragment thereof of the present invention is capable of binding an epitope comprising amino acid residues W36, E39, K40,

C41 and F43 and one or more of C41 and E42 of SEQ ID NO: 2.

In one embodiment an antibody or antigen-binding fragment thereof of the present invention is capable of binding an epitope comprising amino acid residues W36, E39, K40, C41, E42 and F43 of SEQ ID NO: 2.

### SPECIFIC EMBODIMENTS OF THE INVENTION

Aspect 1. An inhibitor capable of specifically binding in the EGF1-3 region of human Protein S for use in the treatment of coagulopathy in a human subject.

2. The inhibitor for use according to aspect 1 wherein said inhibitor is capable of specifically binding in the EGF1 region of human Protein S for use in the treatment of coagulopathy in a human subject.

3. The inhibitor for use according to aspect 1 or 2 wherein the inhibitor is an antibody or antigen-binding fragment thereof.

4. An antibody or antigen-binding fragment thereof capable of specifically binding in the EGF1 region of human Protein S wherein said binding region comprises one or more amino acid residues selected from the group consisting of
W36, E39, K40, C41, E42 and F43 of SEQ ID NO: 2.

5. The antibody or antigen-binding fragment thereof according to aspect 4 wherein said antibody or antigen-binding fragment thereof is capable of specifically binding amino acid residues
W36, E39, K40, and
one or more of amino acid residues C41, E42 and F43 of SEQ ID NO: 2.

6. An antibody or antigen-binding fragment thereof which is capable of specifically binding in the EGF1 region of human Protein S wherein
the light chain of said antibody or antigen-binding fragment comprises
a CDR3 sequence comprising residues 88-96 of SEQ ID NO: 49 (QQWSSIPPT), wherein one or two of said residues can be substituted with a different residue, and
the heavy chain of said antibody or antigen-binding fragment comprises
a CDR3 sequence comprising residues 99-108 of SEQ ID NO: 50 (WGGSGYAMDY), wherein one or two of said residues can be substituted with a different residue.

7. An antibody or antigen-binding fragment thereof according to aspects 6 wherein
the light chain of said antibody or antigen-binding fragment comprises
a CDR1 sequence comprising residues 24-33 SEQ ID NO: 49 (RASSSVSYMY), and/or
a CDR2 sequence comprising residues 49-55 of SEQ ID NO: 49 (ATSNLAS), and/or
a CDR3 sequence comprising residues 88-96 of SEQ ID NO: 49 (QQWSSIPPT)
and the heavy chain of said antibody or antigen-binding fragment comprises
a CDR1 sequence comprising residues 31-35 of SEQ ID NO: 50 (SYWIN), and/or
a CDR2 sequence comprising residues 50-66 of SEQ ID NO: 50 (RIDPYDSETHYAQKFQG), and/or
a CDR3 sequence comprising residues 99-108 of SEQ ID NO: 50 (WGGSGYAMDY).

8. An antibody or antigen-binding fragment thereof according to aspect 6 or 7 wherein
the light chain variable domain (VL) of said antibody or antigen-binding fragment comprises SEQ ID NO: 49,
wherein amino acid residue L45 is substituted with P, and optionally
L46 is substituted with W.
and
the heavy chain variable domain (VH) of said antibody or antigen-binding fragment comprises SEQ ID NO: 50, optionally further comprising one or more of the substitutions selected from a group consisting of M70L, R72V, T74K and V79A.

9. The antibody or antigen-binding fragment thereof according to aspect 6, 7 or 8 wherein the light chain variable domain (VL) of said antibody comprises SEQ ID NO: 51 or 53, and the heavy chain heavy chain variable domain (VH) of said antibody comprises SEQ ID NO: 50, 52, 54 or 55.

10. The antibody or antigen-binding fragment thereof according to aspect 9 wherein the light chain variable domain (VL) of said antibody comprises SEQ ID NO: 51 and the heavy chain variable domain (VH) of said antibody comprises SEQ ID NO: 50.

11. The antibody or antigen-binding fragment thereof according to aspect 9 wherein the light chain variable domain (VL) of said antibody comprises SEQ ID NO: 51 and the heavy chain variable domain (VH) of said antibody comprises SEQ ID NO: 52.

12. The antibody or antigen-binding fragment thereof according to aspect 9 wherein the light chain variable domain (VL) of said antibody comprises SEQ ID NO: 51, and the heavy chain variable domain (VH) of said antibody comprises SEQ ID NO: 54.

13. The antibody or antigen-binding fragment thereof according to aspect 9 wherein the light chain variable domain (VL) of said antibody comprises SEQ ID NO: 51 and the heavy chain variable domain (VH) of said antibody comprises SEQ ID NO: 55.

14. The antibody or antigen-binding fragment thereof according to aspect 9 wherein the light chain variable domain (VL) of said antibody comprises SEQ ID NO: 53 and the heavy chain variable domain (VH) of said antibody comprises SEQ ID NO: 50.

15. The antibody or antigen-binding fragment thereof according to aspect 9 wherein the light chain variable domain (VL) of said antibody comprises SEQ ID NO: 53 and the heavy chain variable domain (VH) of said antibody comprises SEQ ID NO: 52.

16. The antibody or antigen-binding fragment thereof according to aspect 9 wherein the light chain variable domain (VL) of said antibody comprises SEQ ID NO: 53 and the heavy chain variable domain (VH) of said antibody comprises SEQ ID NO: 54.

17. The antibody or antigen-binding fragment thereof according to aspect 9 wherein the light chain variable domain (VL) of said antibody comprises SEQ ID NO: 53 and the heavy chain variable domain (VH) of said antibody comprises SEQ ID NO: 55.

18. The antibody or antigen-binding fragment thereof according to aspects 7 to 17 wherein the heavy chain variable domain (VH) CDR2 amino acid residue D55 of SEQ ID NO: 50 optionally may be substituted with a different amino acid residue which is not C.

19. The antibody according to any one aspects 3 to 18 wherein the antibody is a monoclonal antibody.

20. A polynucleotide which encodes the inhibitor, antibody or antigen-binding fragment thereof according to any one of aspects 1 to 19.

21. A pharmaceutical composition comprising the inhibitor, antibody or antigen-binding fragment thereof or polynucleotide according to any one of aspects 4 to 19 and a pharmaceutically acceptable carrier or diluent.

22. The antibody or antigen-binding fragment thereof according to any one of aspects 4 to 19 for use in the treatment of coagulopathy in a human subject.

23. The antibody or antigen-binding fragment thereof according to aspect 22 for use in the treatment of haemophilia in a human subject.

24. A eukaryotic cell which expresses the inhibitor, antibody or antigen-binding fragment thereof according to any one of aspects 4 to 19.

25. An antibody, or an antigen-binding fragment thereof, which competes with a reference antibody in binding to human Protein S, wherein the reference antibody comprises
a heavy chain variable region and a light chain variable region according to any one of aspects 8 or 18.

### EXAMPLES

### Example 1

### Improvement of APTT by Polyclonal Antibodies Against Protein S in Human Haemophilia Plasma

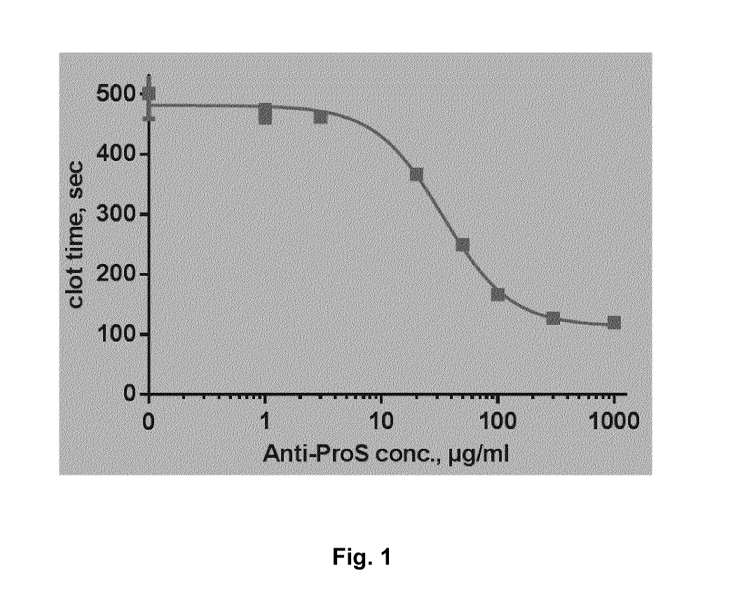

The polyclonal anti-Protein S antibodies concentration-dependently reduced clotting times in the presence of APC in FVIII deficient human plasma (FIG. 1). Congenital FVIII deficient human plasma (George King Biomedical Inc.) were incubated with 0.3 μg/ml APC (Innovative Research) and specified levels of polyclonal anti-Protein S (DAKO #A0384) together with APTT reagent (APTT-SP, IL) for 300 sec at 37° C. prior to re-calicification. Time to fibrin clot formation was measured using an ACL9000 (ILS). The mean EC₅₀ was 37.1 μg/ml (SD=2.4, n=3 experiments) corresponding to app. 250 nM.

### Example 2

### Pro-Coagulant Effect of Anti-Protein S Antibodies Compared with FVIII in Haemophilia A Plasma

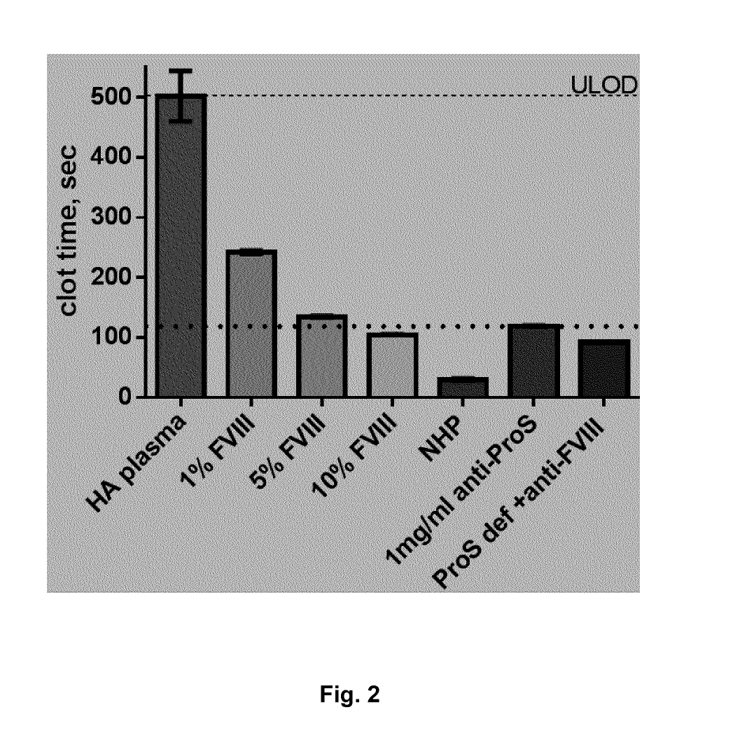

Maximal effect of anti-Protein S antibodies in FVIII deficient plasma is compared with clotting times for normal human plasma and human plasma with 1, 5, and 10% FVIII (in-house), respectively (FIG. 2). The data indicate that full response with anti-Protein S resembles the clotting time for plasma with 5-10% FVIII. The effect of full neutralization of Protein S was confirmed by establishing the clot time for Protein S deficient plasma (Haemochrom Diagnostica) with excess of neutralising FVIII antibodies (in-house) (resembling double Protein S and FVIII deficient plasma).

Plasmas were mixed with APC (0.3 μg/ml), and anti-Protein S (DAKO, #A0384) or FVIII in different combinations together with APTT reagent (APTT-SP, IL) and incubated 300 sec at 37° C. prior to re-calicification. Time to fibrin clot formation was measured using an ACL9000 (ILS). Data are mean±SD, n=3 experiments.

### Example 3

### In Vivo Effect of Polyclonal Antibodies Against Full-Length and Gla-Domain Deleted Mouse Protein S

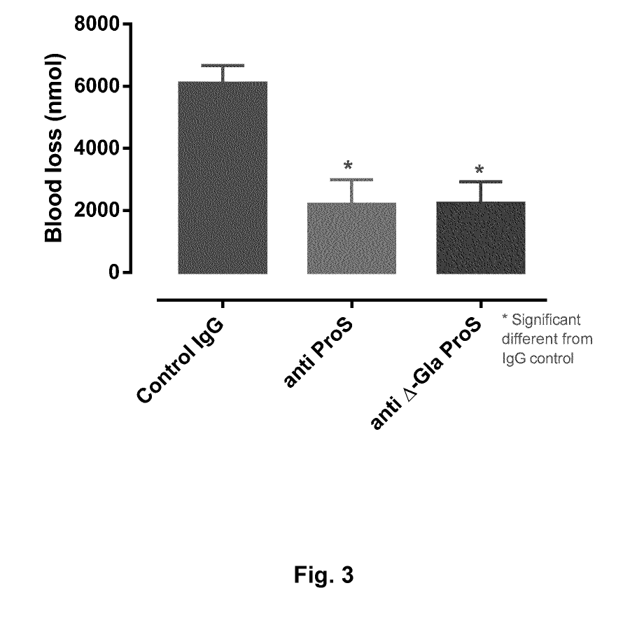

Haemophilia A mice treated with a rabbit polyclonal antibody against full length and desGla-domain mouse Protein S (49 mg/kg, IV), respectively, 5 min before tail clip (4 mm). Blood loss was determined over a 30 min period (Holmberg et al. JTH, 7, 1517-1522 (2006). Data are mean±SEM, n=6-8. Polyclonal antibodies against both Protein S (full length) as well as desGla Protein S significantly reduce the blood loss in the tail bleeding model in haemophilia A mice (FIG. 3).

The rabbit polyclonal antibodies was generated in-house by immunisation of rabbits with full-length and desGla-mouse Protein S, respectively. The rabbit IgG was subsequently purified from plasma.

### Example 4

### Production and Purification Human Protein S Lacking the Gla Domain and the EGF1-4 Domains of Human Protein S

Expression of human desGLA Protein S, SEQ ID NO: 1
Generation of GS-Based Vector for Expression of Human desGLA Protein S

For expression of human desGLA Protein S (SEQ ID NO: 1) in GS-based expression system from Lonza, vector pBOK822 was generated according to the standard procedure described by Lonza and as further outlined below. The expression vector comprises two expression cassettes, one for expression of human desGLA Protein S and a second for expression of the Glutamine synthetase (GS) selection marker.

1. The human desGLA Protein S expression cassette contains:
a. The human cytomegalovirus major immediate early (hCMV-MIE) promoter including the 5′ untranslated sequence from the CMV-MIE locus to facilitate transcription/translation.
b. The cDNA sequence encoding human desGLA Protein S
c. A SV40 polyadenylation signal (SV40 poly-A site)

2. The GS expression cassette contains:
a. The SV40 late promoter
b. The GS mini-gene
c. Two polyadenylation signals (poly-A site1 og 2).

The remaining part of the vector contains the bacterial colE1 replication origin and the ampicillin resistance gene, both for vector propagation in E. coli.
a. The cDNA for human desGLA Protein S was cloned into vector pEE14.4 4 (Lonza) for generation of vector pBOK822, by transfer of a PmeI/BsiWI restriction fragment from existing pTT-based vector pJSV320, into NruI/BsiWI linearized pEE14.4 vector.

The original pTT-based vector was generated by PCR amplification of the human Protein S cDNA 3′ of the GLA domain at the position corresponding to the N-terminus of EGF1 using full length human Protein S, IMAGE clone ID 3909023 as template. The amplified fragment was inserted by standard restriction digest/ligation into a pTT-based vector carrying the signal peptide for human CD33 and an HPC4 purification tag. The human desGLA protein S cDNA was inserted in-frame with both the 5′ CD33 signal peptide sequence and with the 3′ HPC4 tag sequence including an Ala-Leu-Ala (ALA) cloning spacer (residues 564-578 of SEQ ID NO:1).
b. The sequence of the final vector pBOK822 was verified by sequencing of the human desGLA Protein S insert.
c. In preparation for transfections the vector pBOK822 was linearized by AclI restriction digest and isolated using the QIAEX II Gel extraction kit (Qiagen).

Human desGLA Protein S production cell line development
1. CHOK1SV cells were transfected with linearized human desGLA Protein S GS expression vector pBOK822 by electroporation and seeded at limited densities into twenty 96-well plates according to the standard protocol from Lonza.
2. Transfected cells were incubated in glutamine-free CD CHO (Gibco) medium containing either 25 μM or 37 μM methionine sulphoximine (MSX) (Sigma); a Glutamine Synthetase (GS) selective inhibitor. Clones were identified after ˜3 weeks by visual inspection of the plates.
3. 24 selected clones were expanded from 96-well stationary cultures to 24-well stationary cultures in CD CHO medium containing 25 μM MSX.
4. Individual clones were ranked and selected based on the accumulated human desGLA Protein S yield over 7 days in 24-well stationary cultures. Protein S yields were measure by spot-blot/Western blot analysis below and the best 3 clones chosen for further analysis:
   a. 5 μl cell culture was spotted onto a nitro cellulose membrane and allowed to dry.
   b. The membrane was blocked for 2 min in TBS containing 2% v/v Tween-20.
   c. The membrane was transferred to TBS containing 0.1% v/v Tween-20 and 1:1000 dilution of polyclonal rabbit Anti-Protein C (HPC4)-tag antibody (Genscript) and incubated at room temperature for 60 min.
   d. The membrane was washed 3× for 5 min in TBS containing 0.1% v/v Tween-20.
   e. The membrane was transferred to TBS containing 0.1% v/v Tween-20 and 1:10000 dilution of fluorescently labelled anti-rabbit Ig antibody (Licor) and incubated at room temperature for 60 min.
   f. The membrane was washed 3× for 5 min in TBS containing 0.1% v/v Tween-20 and scanned using an Odyssey Imaging system (Licor).
5. Selected cell lines were expanded from 24-well stationary cultures to 5 ml shaker cultures in 50 ml bioreactor tubes (TTP) followed by expansion to 30 ml cultures in 125 ml Erlenmeyer flasks (Corning). At this stage the selection pressure is kept at 25 μM MSX. The highest producing human desGLA Protein S cell line was selected based on the accumulated Protein S yields over 7 days in shaker cultures (over-growth (OG) culture). Protein S yields were measure by standard Western blot analysis.
   a. Supernatants were analyzed by SDS-PAGE, followed by standard Western Blot analysis according to the protocol described above for the spot blot/western blot analysis.
6. Final cell line chosen for production of human desGLA Protein S was: BRTK822_25_2_C10.
7. For production a culture of BRTK822_25_2_C10 was expanded and seeded into a 2×1 L cultures in CD CHO medium containing 25 μM MSX and incubated for 7 days in 3 L Erlenmeyer flasks in an orbital shaker at 36.5° C., 8% CO₂ and 85-125 rpm.
8. After 7 days the supernatant was harvested by centrifugation, followed by filtration using 0.22 μm PES filter units (Corning).

### Expression of Human Protein S EGF1-4, SEQ ID NO: 2

### Generation of GS-Based Vector for Expression of Human Protein S EGF1-4

For expression of human Protein S EGF1-4 (SEQ ID NO: 2) in GS-based expression system from Lonza, vector pBOK821 was generated according to the standard procedure described by Lonza and as further outlined below. The expression vector comprises two expression cassettes, one for expression of human Protein S EGF1-4 and a second for expression of the Glutamine synthetase (GS) selection marker.

1. The human Protein S EGF1-4 expression cassette contains:
a. The human cytomegalovirus major immediate early (hCMV-MIE) promoter including the 5′ untranslated sequence from the CMV-MIE locus to facilitate transcription/translation.
b. The cDNA sequence encoding human Protein S EGF1-4.
c. A SV40 polyadenylation signal (SV40 poly-A site).

2. The GS expression cassette contains:
a. The SV40 late promoter
b. The GS mini-gene
c. Two polyadenylation signals (poly-A site 1 and 2).

The remaining part of the vector contains the bacterial colE1 replication origin and the ampicillin resistance gene, both for vector propagation in E. coli.
d. The cDNA for human Protein S EGF1-4 was cloned into vector pEE14.4 (Lonza) for generation of vector pBOK821, by transfer of a PmeI/EcoRI restriction fragment from existing pTT-based vector (pJSV321) into NruI/EcoRI linearized pEE14.4 vector.

The original pTT-based vector was generated by PCR amplification of the human Protein S cDNA covering the EGF1-4 domains using full length human Protein S, IMAGE clone ID 3909023 as template. The amplified fragment was inserted by standard restriction digest/ligation into a pTT-based vector carrying the signal peptide for human CD33 and an HPC4 purification tag. The human protein S EGF1-4 cDNA was inserted in-frame with both the 5′ CD33 signal peptide sequence and with the 3′ HPC4 tag sequence including an Ala-Leu-Ala cloning spacer (residues 174-188 SEQ ID NO: 2).
e. The sequence of the final vector pBOK821 was verified by sequencing of the human Protein S EGF1-4 insert.
f. In preparation for transfections the vector pBOK821 was linearized by AclI restriction digest and isolated using the QIAEX II Gel extraction kit (Qiagen).

### Human Protein S EGF1-4 Production Cell Line Development

1. CHOK1SV cells were transfected with linearized human Protein S EGF1-4 GS expression vector pBOK821 by electroporation and seeded at limited densities into twenty 96-well plates according to the standard protocol from Lonza.
2. Transfected cells were incubated in glutamine-free CD CHO (Gibco) medium containing either 25 μM or 37 μM methionine sulphoximine (MSX) (Sigma); a Glutamine Synthetase (GS) selective inhibitor. Clones were identified after ˜3 weeks by visual inspection of the plates.
3. 24 selected clones were expanded from 96-well stationary cultures to 24-well stationary cultures in CD CHO medium containing 25 μM MSX.
4. Individual clones were ranked and selected based on the accumulated human Protein S EGF1-4 yield over 7 days in 24-well stationary cultures. Protein yields were measure by spot-blot/Western blot analysis below and the best 3 clones chosen for further analysis:
   a. 5 μl cell culture was spotted onto a nitro cellulose membrane and allowed to dry.
   b. The membrane was blocked for 2 min in TBS containing 2% v/v Tween-20.
   c. The membrane was transferred to TBS containing 0.1% v/v Tween-20 and 1:1000 dilution of polyclonal rabbit Anti-Protein C (HPC4)-tag antibody (Genscript) and incubated at room temperature for 60 min.
   d. The membrane was washed 3× for 5 min in TBS containing 0.1% v/v Tween-20.
   e. The membrane was transferred to TBS containing 0.1% v/v Tween-20 and 1:10000 dilution of fluorescently labelled anti-rabbit Ig antibody (Licor) and incubated at room temperature for 60 min.
   f. The membrane was washed 3× for 5 min in TBS containing 0.1% v/v Tween-20 and scanned using an Odyssey Imaging system (Licor)
5. Selected cell lines were expanded from 24-well stationary cultures to 5 ml shaker cultures in 50 ml bioreactor tubes (TTP) followed by expansion to 30 ml cultures in 125 ml Erlenmeyer flasks (Corning). At this stage the selection pressure is kept at 25 μM MSX. The highest producing human Protein S EGF1-4 cell line was selected based on the accumulated Protein S yields over 7 days in shaker cultures (over-growth (OG) culture). Protein S yields were measure by standard Western blot analysis.
   a. Supernatants were analyzed by SDS-PAGE, followed by standard Western Blot analysis according to the protocol described above for the spot blot/western blot analysis.
6. Final cell line chosen for production of human Protein S EGF1-4 was: BRTK821_37_2_B11.
7. For production a culture of BRTK821_37_2_B11 was expanded and seeded into a 2×1 L cultures in CD CHO medium containing 25 μM MSX and incubated for 7 days in 3 L Erlenmeyer flasks in an orbital shaker at 36.5° C., 8% CO₂ and 85-125 rpm.
8. After 7 days the supernatant was harvested by centrifugation, followed by filtration using 0.22 μm PES filter units (Corning).

### Purification

The recombinant human desGLA Protein S and human Protein S EGF1-4 were purified by affinity chromatography (anti-HPC4-Sepharose) followed by gelfiltration on a Superdex 75 column. The final purity of human Protein S EGF1-4 was estimated to be 100% by SDS-PAGE and SEC-HPLC. Endotoxin was <0.1 EU/mg (measured by Kinetic turbidimetric test). For human desGLA Protein S, the purity was 95% and endotoxin <0.04 EU/mg.

### Example 5

### Expression and Purification of Full Length Cynomolgus Monkey Protein S, SEQ ID NO: 3

Generation of pQMCF1 Vector for Expression of Cynomolgus Protein S

The QMCF expression platform from Icosagen was used for expression of cynomolgus monkey (Macaca fascicularis) Protein S (SEQ ID NO: 3).
1. The QMCF CHO cell line, CHOEBNAL85 supports stable maintenance and partitioning of the accompanying QMCF plasmids.
2. The QMCF plasmids contain:
   a. the mouse polyomavirus (Py) DNA replication origin which in combination with
   b. the Epstein-Barr virus (EBV) EBNA-1 protein binding site ensures stable propagation of plasmids in the QMCF cells.

The QMCF based expression vector for expression of full length cynomolgus Protein S was generated through a series of steps:
a. The cDNA for cynomolgus Protein S was cloned from Macaca fasicularis cDNA using amplification primers designed based on the sequences for accession XM_005548385.
b. The amplified fragment was purified and cloned into Zero-BLUNT topo vector (Invitrogen) for sequence verification.
c. For the final expression vector pBOK835, the cynomolgus Protein S was amplified using adaptor primers introducing 1) a Kozak sequence motif (GCCGCCACC) 5′ of the ATG start codon and a 5′ terminal NotI restriction site, 2) an HPC4 tag at the C-terminus of the Protein S sequence (residue 636-647 of SEQ ID NO: 3 and a 3′ terminal EcoRI restriction site.
d. The resulting PCR fragment was purified and used as template for a secondary PCR amplification using a second set of adaptor primers introducing, 1) a terminal NheI restriction site 5′ of the Kozak sequence and ATG start codon and 2) a terminal AscI restriction site 3′ of the HPC4 tag sequence.
e. From the resulting PCR fragment, a NheI/AscI restriction fragment was generated and inserted into NheI/AscI linearized pQMCF1 vector.
f. The sequence of the final vector pBOK835 was verified by sequencing of cynomolgus Protein S insert.

### Transfection/Expression of Full Length Cynomolgus Protein S

1. CHOEBNALT85 cells were maintained in QMix1 medium prepared from equal amounts of CD CHO Medium (Gibco) and 293 SFM II Medium (Gibco) supplemented with 6 mM L-Glutamine (Gibco), 0.5×HT Supplement (Gibco) and 20 μg/ml puromycin (Gibco).
2. Cells were harvested, washed and resuspended in CH CHO medium (10E7 cells in 0.7 ml) before transfection with 10 μg cynomolgus Protein S pQMCF1 expression vector (pBOK835) by electroporation using a Gene Pulser XceII™ Electroporation System (Biorad) and an exponential electroporation protocol (300V, 900 μF, ∞Ω, 4 mm cuvette).
3. Immediately after electroporation the cells were transferred to 20 ml QMix1 medium in a 125 ml Erlenmeyer flask and incubated in an orbital shaker at 36.5° C., 8% CO₂, 125 rpm.
4. 24 hrs post transfection G418 selection reagent (Gibco) was added to a final concentration of 700 μg/ml and the cell were left to recover for 72-96 hrs. Recovery was monitored by measuring cell culture viability and density using a Cedex HiRes Cell Counter.
5. When cells again were actively dividing, the culture was expanded to reach the final production volume, maintaining cells between 0.2×10E6-3×10E6 cells/mi.
6. For final production, 2×1 L cultures was seeded into 3 L Erlenmeyer flasks in QMix1 medium supplemented with 700 ug/ml G418 and 5 ug/ml Vitamin K and incubated for 7 days in an orbital shaker at 36.5° C., 8% CO₂ and 85 rpm.

After 7 days the supernatant was harvested by centrifugation, followed by filtration using 0.22 μm PES filter units (Corning). The purification was done as describe in the example above. Final purity of cynomolgus Protein S was estimated to be high by SDS-PAGE, N-terminal amino acid sequence analysis and LC-MS, however, monomeric fractions measured by SEC-HPLC was 48%. Endotoxin was 63 EU/mg.

### Example 6

### Generation of Anti-Protein S (EGF1-4) Monoclonal Antibodies

RBF mice were immunized with Protein S derived from human plasma (HTI), recombinant human Protein S lacking the Gla domain (desGLA Protein S SEQ ID NO: 1) or recombinant protein comprising only the EGF1-4 domains of human Protein S (SEQ ID NO: 2). Protein was emulsified in incomplete Freund's adjuvants prior to immunization. Mice were injected subcutaneously at immunization start followed by three bi-weekly intraperitoneal immunizations. Blood was collected from mice 10 days after the last immunization and serum was prepared and the anti-EGF1-4 antibody titres were determined by ELISA in which NUNC Maxisorp plates were coated with EGF1-4 domains of human Protein S and blocked before diluted serum was applied. After incubation and washing a HRP-labelled goat-anti-mouse IgG secondary antibody (Jackson) was added and the ELISA was developed after incubation and wash by addition of 3,3′,5,5′-Tetramethylbenzidine.

Anti-EGF1-4 responding mice were boosted intravenously (i.v.) with desGLA Protein S or EGF1-4 domains of human Protein S without adjuvant. The spleen was removed aseptically three days after boost and dispersed to a single cell suspension. Fusion of mouse spleen cells and myeloma cells (P3X63Ag8.653, ATCC-# CRL1580) was done by standard electrofusion and cells were seeded in microtiter plates and cultured at 37° C., 5% CO₂. The tissue-culture medium was changed two times over a period of 13 days and hybridomas were selected in HAT/HT medium (Sigma).

Antibodies binding to Protein S and Protein S fragments can also be identified by screening of FAb, scFv etc. libraries by phage display. Pro-coagulant Protein S binders may also be obtained by screening of peptide libraries by phage display or aptamer libraries.

### Example 7

### Primary Screening for Antibodies Binding to EGF1-4 Domains of Human Protein S and Cynomolgus Monkey Protein S

Hybridoma supernatants were analysed for the ability to bind human EGF1-4 in ELISA as described above and subsequent to recombinant Protein S from cynomolgus monkey (SEQ ID NO: 3). Antibodies binding to both the EGF1-4 domains of human Protein S and cynomolgus monkey Protein S was expressed and purified from hybridoma supernatant prior to functional characterization. In order to generate a monoclonal and stable hybridoma cell line, hybridoma cells were sub-cloned by limited dilution. Cells were seeded into 96 well plates by a density of 1 cell/well. After two weeks, supernatants from each well were screened for binding to EGF1-4 domains of human Protein S as described above.

### Example 8

### Screening for Anti-Protein S (EGF1-4) Mediated Protection of ACP/Protein S Inactivation of FVa

The neutralising effect of the Protein S binding antibodies (typically present in the FVa inactivation reaction step at concentrations ranging from 0-400 nM) on Protein S cofactor activity on APC-mediated inactivation of FVa were measured in a biochemical assay at room temperature.

Briefly, 30 μL of purified antibody (in 20 mM Tris, pH 7.4) were mixed with 20 μL human Protein S (Haematologic Technologies Inc, #HCPS-0090) in assay buffer (30 mM HEPES, 135 mM NaCl, 1 mM EDTA, 0.1% BSA, pH 7.4) in a microtiter plate (Perkin Elmer, #6005659). The reaction was incubated for 30 min to allow antigen binding. Then, 20 μL of a mixture containing human APC (Haematologic Technologies Inc, #HCAPC-0080) and phospholipids-TGT (Rossix, #PL604T) were added, and the reaction was incubated at for 5 min. Subsequently, 20 μL of human factor Va (Haematologic Technologies Inc, #HCVA-0110) was added, and the inactivation reaction was allowed to proceed for 30 min. At this step, the concentration of Protein S was 10 nM, APC was 65 pM, and FVa was 50 pM. Then, 100 μL of a mixture of both human prothrombin (Enzyme Research Laboratories, #HP 1002) and human FXa (Enzyme Research Laboratories, #HFXa 1011) was added to initiate thrombin generation under which FVa was the rate limiting determinant. The reaction proceeded for 10 min. At this step, the concentration of the phospholipids was 23.8 μM, prothrombin was 100 nM, and FXa was 0.5 nM. Finally, 100 μL of the chromogenic thrombin peptide substrate S-2238 (Chromogenix, #S-2238) dissolved in EDTA buffer (20 mM HEPES, 140 mM NaCl, 20 mM EDTA, 1 g/L BSA, pH 7.4) was added to a final concentration of 400 μM, and the plate was read immediately and repeatedly at 405 nm every 30 sec for 10 min. The initial reaction velocities were calculated for each antibody concentration and used as a measure of remaining FVa cofactor activity. This signal was normalized according to two controls not containing any antibody; both containing FVa and APC, but +/−Protein S. Thus, 0% corresponds to the signal in the presence of Protein S, and 100% corresponds to the signal in the absence of Protein S. Antibodies that in a concentration-dependent way could restore FVa cofactor activity to, or above, 30% of maximal cofactor activity were considered Protein S functionally neutralising. The antibodies fulfilling the criteria was cloned and further investigated as described in the following examples

### Example 9

### ELISA Binding of Monoclonal Antibodies Purified from Hybridoma Supernatants to Protein S and Variants Hereof

Binding of antibodies purified from the hybridoma supernatants EGF1-4 domains of human Protein S and cynomolgus monkey Protein S was confirmed in and ELISA (see example 6). Furthermore, binding to plasma derived Protein S (HTI) and the EGF1-2 and EGF3-4 domains of human Protein S were investigated (table 1). All binding experiments were performed in calcium-free TBS buffer (138 μM NaCl, 270 nM KCl, pH 8, Sigma T6664). Thus, the identified anti-Protein S mAbs were capable of binding Protein S in a calcium independent manner.

TABLE 1
ELISA binding of monoclonal antibodies purified from hybridoma supernatants

| Hybridoma name | hEGF1-4 | hEGF1-2 | hEGF3-4 | Plasma PS | hPS-dGla | CyPS-dGla |
|---|---|---|---|---|---|---|
| M-hProtS-2F188A1 | + | + | − | + | + | + |
| M-hProtS-2F380A1 | + | + | − | + | + | + |
| M-hProtS-2F382A1 | + | + | − | + | + | + |
| M-hProtS-2F82A1 | + | + | − | + | + | + |
| M-hProtS-2F4A1 | + | + | − | + | + | + |
| M-hProtS-3F2A1 | + | + | − | + | + | + |
| M-hProtS-3F38A2 | + | − | − | + | + | + |
| M-hProtS-3F62A5 | + | − | − | + | + | + |
| M-hProtS-6F101A3 | + | + | − | + | + | + |
| M-hProtS-6F120A1 | + | − | − | + | + | + |
| M-hProtS-6F128A2 | + | + | − | + | + | + |
| M-hProtS-6F138A3 | + | − | − | + | + | + |
| M-hProtS-6F151A2 | + | + | − | + | + | + |
| M-hProtS-6F153A2 | + | − | − | + | + | + |
| M-hProtS-6F170A2 | + | − | + | + | + | + |
| M-hProtS-6F206A1 | + | + | − | + | + | + |
| M-hProtS-6F216A3 | + | + | − | + | + | + |
| (mAb 0914) | | | | | | |
| M-hProtS-6F265A1 | + | + | − | − | + | + |
| Anti-TNP | − | − | − | − | − | − |

hPS-dGla: Gla-domain deleted recombinant human Protein S; hEGF1-4: EGF domain 1 to 4 of recombinant human Protein S; hEGF1-2: EGF domain 1 and 2 of recombinant human Protein S; hEGF3-4: EGF domain 3 and 4 of recombinant human Protein S; CyPS-dGla: Gla-domain deleted recombinant cynomolgus Protein S. Anti-TNP: Mouse anti-TNP negative ctrl mAb. ‘+’: binding, ‘−’: no binding.

The binding data was subsequent repeated for a subset of the molecular cloned antibodies (described in the example below; table 2).

TABLE 2
ELISA binding of recombinant anti-Protein S antibodies

| Molecular cloned NNCD# | hEGF1-4 | hEGF1-2 | hEGF3-4 | Plasma PS | hPS-dGla | CyPS-dGla |
|---|---|---|---|---|---|---|
| 0322-0000-0916 | + | + | − | + | + | + |
| 0322-0000-0918 | + | + | − | + | + | + |
| 0322-0000-0909 | + | + | − | + | + | + |
| 0322-0000-0920 | + | + | − | + | + | + |
| 0322-0000-0912 | + | + | − | + | + | + |
| 0322-0000-0921 | + | + | − | + | + | + |
| 0322-0000-0910 | + | − | + | + | + | + |
| 0322-0000-0914 | + | + | − | + | + | + |
| 0322-0000-0919 | + | − | + | + | + | + |
| Anti-TNP | − | − | − | − | − | − |

hPS-dGla: Gla-domain deleted recombinant human Protein S; hEGF1-4: EGF domain 1 to 4 of recombinant human Protein S; hEGF1-2: EGF domain 1 and 2 of recombinant human Protein S; hEGF3-4: EGF domain 3 and 4 of recombinant human Protein S; CyPS-dGla: Gla-domain deleted recombinant cynomolgus Protein S. Anti-TNP: Mouse anti-TNP negative ctrl mAb.

### Example 10

### Cloning and Sequencing of Anti-Protein S (EGF1-4) Monoclonal Antibodies Variable Light and Variable Heavy Chains cDNA from Isolated Hybridomas

This example describes cloning and sequencing of the murine heavy chain and light chain sequences of anti-Protein S antibodies listed in table 1.

Total RNA was extracted from hybridoma cells using the RNeasy-Mini Kit from Qiagen and used as template for cDNA synthesis. cDNA was synthesized in a 5′-RACE reaction using the SMART™ RACE cDNA amplification kit from Clontech. Subsequent target amplification of HC and LC sequences was performed by PCR using Phusion Hot Start polymerase (Finnzymes) and the universal primer mix (UPM) included in the SMART™ RACE kit as forward primer.

A reverse primer with the following sequence was used for HC (VH domain) amplification (SEQ ID NO: 47):

5′-CCCTTGACCAGGCATCCCAG-3′

A reverse primer with the following sequence was used for LC amplification (SEQ ID NO: 48):

5′-GCTCTAGACTAACACTCATTCCTGTTGAAGCTCTTG-3′

PCR products were separated by gel electrophoresis, extracted using the GFX PCR DNA & Gel Band Purification Kit from GE Healthcare Bio-Sciences and cloned for sequencing using a Zero Blunt TOPO PCR Cloning Kit and chemically competent TOP10 E. coli (Invitrogen). DNA plasmid material, for sequencing was obtained from plasmid preparations generated by a standard alkaline lysis protocol using a DNA miniprep kit from Qiagen. Alternatively DNA material for sequencing was obtained from colony PCR reactions, performed on selected colonies using an AmpliTaq Gold Master Mix from Applied Biosystems and M13uni/M13rev primers. Colony PCR clean-up was performed using the ExoSAP-IT enzyme mix (USB). Sequencing was performed at MWG Biotech, Martinsried Germany using M13uni(−21)/M13rev(−29) sequencing primers. Sequences were analyzed and annotated using the VectorNTI program. All kits and reagents were used according to the manufacturer's instructions.

A single unique murine kappa type LC and a single unique murine HC, subclass mIgG1 was identified for each of the hybridomas.

Amino acid sequences for the variable heavy chain and variable light chain sequences are specified as SEQ IDs NO: 4-45 (the leader peptide sequences are not included), cf. also section ‘Brief description of sequences’ above. CDR sequences are annotated and highlighted in FIGS. 9 and 10.

### Example 11

### Recombinant Expression of Anti-Protein S Antibodies

### Generation of Vectors for Recombinant Expression of Anti-Protein S Antibodies:

A series of CMV promoter-based expression vectors (pTT vectors) were generated for transient expression of mouse IgG1 and mouse/human IgG4(S241P) chimeric anti-Protein S antibodies in EXPI293F cells (Life Technologies) (0322-0000-1069). The pTT vectors are developed for transient protein expression by Yves Durocher (Durocher et al. Nucleic Acid Research, 2002). In addition to the CMV promoter, the pTT-based vectors contain a pMB1 origin, an EBV origin and the Amp resistance gene.

### Light Chain (LC) Expression Vectors:

pTT-based LC vectors were generated for transient expression of mouse anti-Protein S antibodies. Initially for each anti-Protein S antibody (see table 1), the region corresponding to the variable light chain (VL) domain of the antibody was PCR amplified from an original TOPO sequencing clone, using primers containing sequences specific for the 3′ and 5′ region of the identified variable domain sequences. In addition, the sense primer contained a sequence complementary to the DNA sequence of the 3′ end of the human CD33 signal peptide sequence. Correspondingly, the anti-sense primer contained a sequence complementary to the DNA sequence of the 5′ end of the light chain constant region. The generated PCR fragment was purified using the GFX PCR Purification Kit (GE Healthcare) and cloned into a PCR amplified fragment of a pTT-based vector containing the CD33 signal peptide sequence and the sequence for a mouse kappa constant region (for mouse antibody expression) or the sequence for a human kappa constant region (for chimeric antibody expression). The vector fragment was obtained by PCR amplification of the pTT vector using an anti-sense primer specific for the 3′ end of the human CD33 signal peptide sequence and a sense primer specific for the 5′ end of the light chain constant region. The vector fragment was treated with DpnI restriction nuclease to remove template DNA and purified using the GFX PCR Purification Kit (GE Healthcare). The amplified VL fragment was cloned in to the vector in-frame between the CD33 signal peptide and the light chain constant region using the In-Fusion® HD Cloning Kit (Clontech) according to manufacturer's instructions. The cloning reaction was subsequently transformed into E. coli for selection. The sequences of the final constructs were verified by DNA sequencing.

### Heavy Chain (HC) Expression Vectors:

pTT-based HC vectors were generated for transient expression of mouse anti-Protein S antibodies. Initially for each anti-Protein S antibody (see table 1), the region corresponding to the variable heavy chain (VH) domain of the antibody was PCR amplified from an original TOPO sequencing clone, using primers containing sequences specific for the 3′ and 5′ region of the identified variable domain sequences. In addition, the sense primer contained a sequence complementary to the DNA sequence of the 3′ end of the human CD33 signal peptide sequence. Correspondingly, the anti-sense primer contained a sequence complementary to the DNA sequence of the 5′ end of the heavy chain constant region. The generated PCR fragment was purified using the GFX PCR Purification Kit (GE Healthcare) and cloned into a PCR amplified fragment of a pTT-based vector containing the CD33 signal peptide sequence and sequence for a mouse IgG1 constant region (for mouse antibody expression) or the sequence for a human IgG4(S241P) constant region (for chimeric antibody expression). The proline mutation at position 241 (numbering according to Kabat, corresponding to residue 228 per the EU numbering system (Edelman G. M. et al. Proc. Natl. Acad. USA 63, 78-85 (1969)) was introduced in the IgG4 hinge region to eliminated formation of monomeric antibody fragments, i.e., “half-antibodies” comprised of one LC and one HC.

The vector fragment was obtained by PCR amplification of the vector sequence using an anti-sense primer specific for the 3′ end of the human CD33 signal peptide sequence and a sense primer specific for the 5′ end of the heavy chain constant region. The vector fragment was treated with DpnI restriction nuclease to remove template DNA and purified using the GFX PCR Purification Kit (GE Healthcare). The amplified VH fragment was cloned in to the vector in-frame between the CD33 signal peptide and the heavy chain constant region using the In-Fusion® HD Cloning Kit (Clontech) according to manufacturer's instructions. The cloning reactions were subsequently transformed into E. coli for selection. The sequence of the final constructs was verified by DNA sequencing.

### Recombinant Expression of Monoclonal Antibodies:

The anti-Protein S antibodies were expressed transiently in EXPI293F cells (Life Technologies) by co-transfection of the pTT-based LC/HC expression vectors according to manufacturer's instructions. The following procedure describes the generic EXPI293F expression protocol.

### Cell Maintenance:

EXPI293F cells were grown in suspension in Expi293™ expression medium (Life Technologies). Cells were cultured in Erlenmeyer shaker flasks in an orbital shaker incubator at 36.5° C., 8% CO2 and 85-125 rpm and maintained at cell densities between 0.4-4×106 cells/ml.

### DNA Transfection:

1) Separate dilutions of DNA and transfection reagent are initially prepared.
   a) Use a total of 1 μg of vector DNA (0.5 μg LC vector and 0.5 μg HC vector) per ml cell culture. Dilute the DNA in Opti-MEM media (Gibco) 50 μl medium/μg DNA, mix and incubate at room temperature (23-25° C.) for 5 min.
   b) Use Expifectamin™ 293 (Life Technologies) as transfection reagent at a concentration of 2.7 μl per μg DNA. Dilute the Expifectamin™ solution 18.5× in Opti-MEM media (Gibco), mix and incubate at room temperature (23-25° C.) for 5 min.
2) Mix DNA and Expifectamin™ 293 dilutions and leave to incubate at room temperature (23-25° C.) for 10 min.
3) Add the DNA-Expifectamin™ 293 mix directly to the EXPI293F cell culture.
   a) At the time of transfection the cell density of the EXPI293F culture should be 2.8-3.2×106 cells/ml.
4) Transfer the transfected cell culture to an orbital shaker incubator at 36.5° C., 8% CO₂ and 85-125 rpm.
5) 18 hrs post transfection, add 5 ul Expifectamin™ 293 Transfection Enhancer1/ml culture and 50 μl Expifectamin™ 293 Transfection Enhancer2/ml culture and return culture to an orbital shaker incubator at 36.5° C., 8% CO2 and 85-125 rpm.
6) 5 days post transfection, cell culture supernatants were harvested by centrifugation, followed by filtration through a 0.22 μm PES filter unit (Corning).

### Example 12

### Identification of Neutralizing Anti-Protein S Antibodies in Haemophilic Plasma by Thrombin Generation Assay

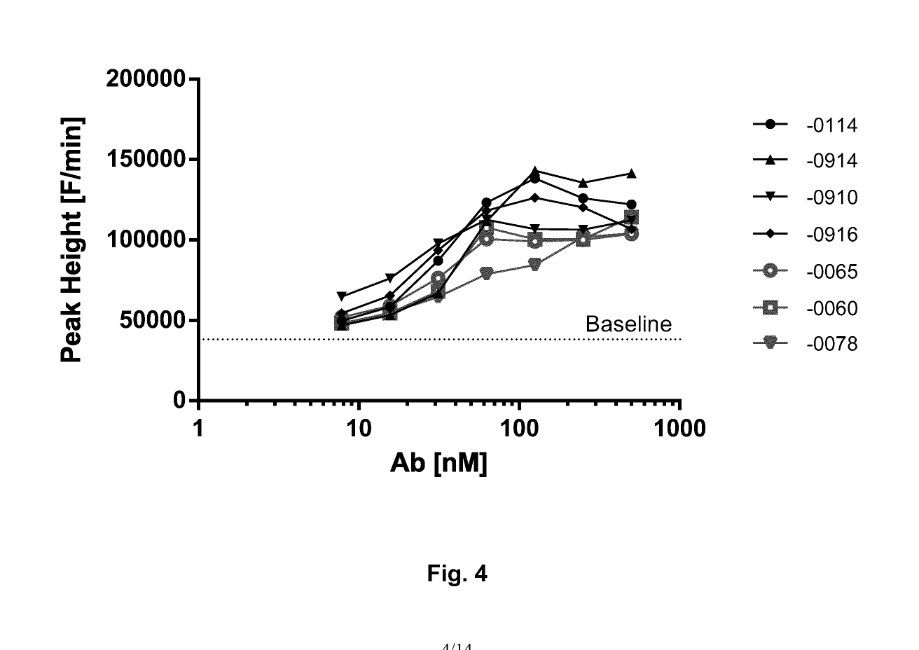

Anti-Protein S antibodies were identified as being capable of increasing thrombin generation in the presence of exogenously added APC in a plasma-based thrombin generation assay. The purified test antibodies were tested in the final assay at 0 nM-500 nM at room temperature. Briefly, human haemophilia A (HA) (FVIII deficient) plasma (Georg King Medical, #0800) stored at −80° C. was thawed in water at 37° C. for 5 min, and then stored at room temperature until use. 18 μL plasma was added to a 384-well microtiter plate (Perkin Elmer), and then 2 μL antibody solution (in 20 nM Tris, pH 7.4) was added, and the antigen binding proceeds for 20 min. Subsequently, 5 μL of a solution, where APC (Haematologic Technologies Inc, #HCAPC-0080) was spiked (100-fold dilution) into a prepared PPP-Reagent LOW reagent (Thrombinoscope, #TS31.00), was added to the assay, which ultimately resulted in APC at 2 nM, tissue factor at 1 pM and phospholipids at 4 μM in the final assay. Without incubation, 5 μL of a prepared FluCa reagent (Thrombinoscope, #TS50.00) was added, and a continuous reading of fluorescence was done every 30 sec for 2 hrs. The thrombogram was calculated as the first derivative of the integral fluorescence curve, and the ETP and peak thrombin parameters were calculated from the thrombogram, and used in the evaluation of thrombin generation. Certain commercial monoclonal antibodies (table 3) were compared to the performance of a subset of the following in-house anti-Protein S antibodies: 0322-0000-0114, 0322-0000-0914, 0322-0000-0910, 0322-0000-0916 and buffer-only (FIG. 4).

TABLE 3
Source of select commercial monoclonal Protein S antibodies

| NNC# | Vendor | Clon/lot no. |
|---|---|---|
| 0322-0000-0060 | Haematologic | AHPS-5091/Y0220-0.1MG |
| | Technologies Inc | |
| 0322-0000-0065 | US Biological (BioSite) | 8.H.9/L9073061 |
| 0322-0000-0078 | Abcam | Unknown/GR60196 |
| | | Product id: ab61364 |

### Example 13

### Effect of Antibodies in Thrombin Generation in Human, Cynomolgus Monkey and Rabbit Plasma Measured by Calibrated Automated Thrombography

### Thrombin Generation in Human Haemophilia A Plasma

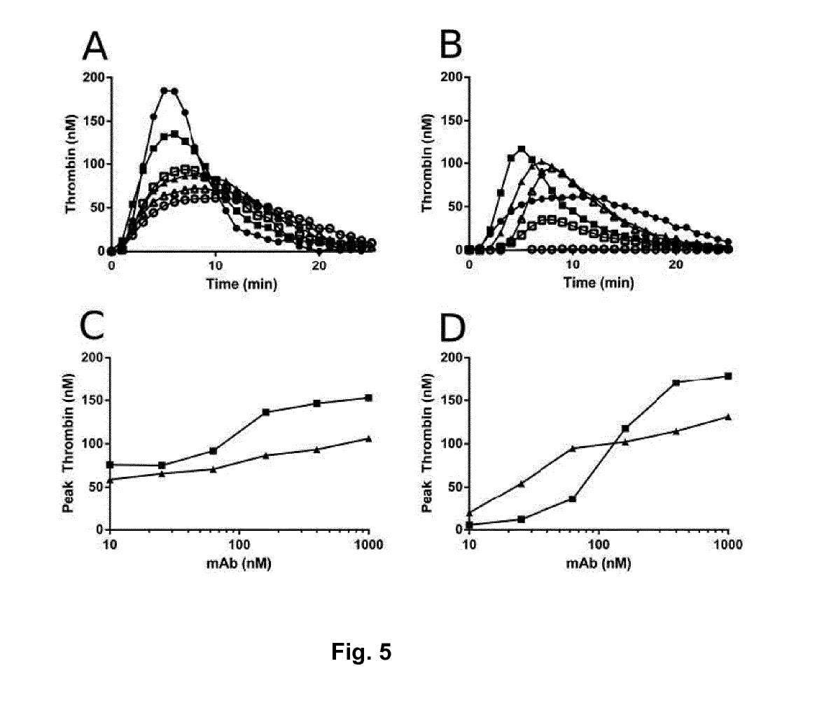

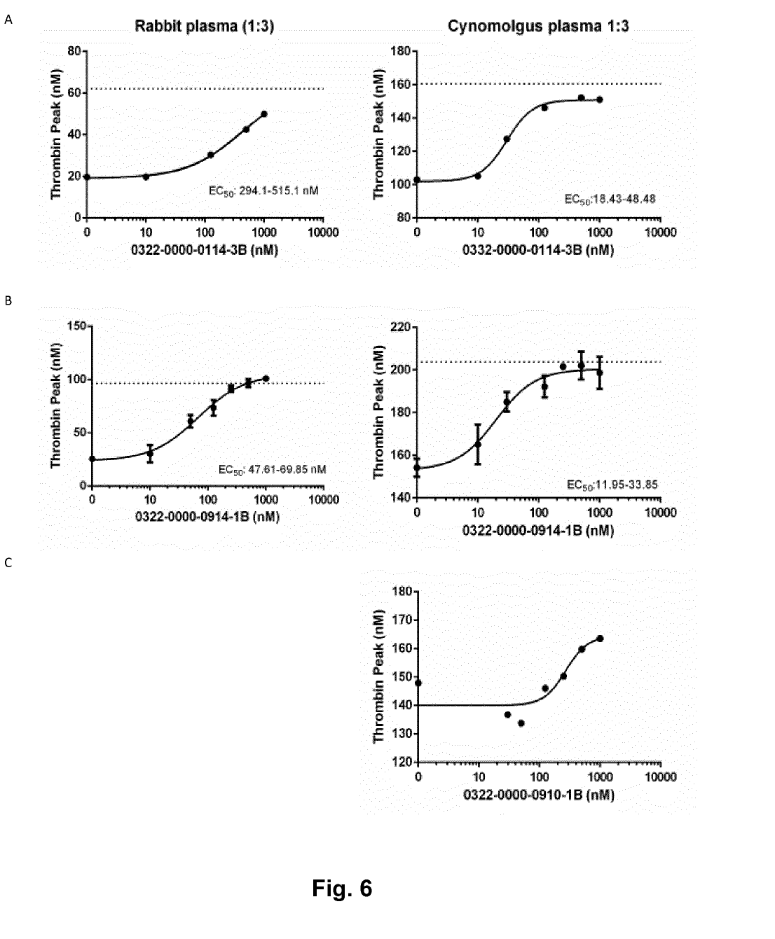

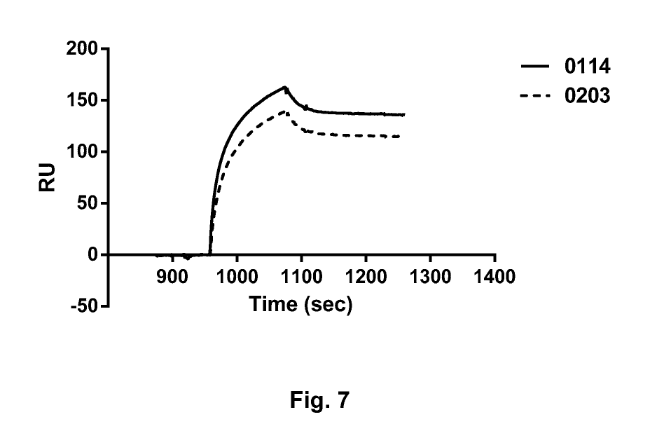

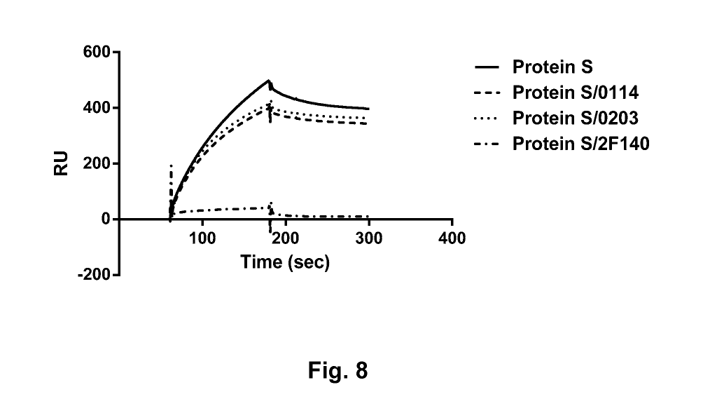

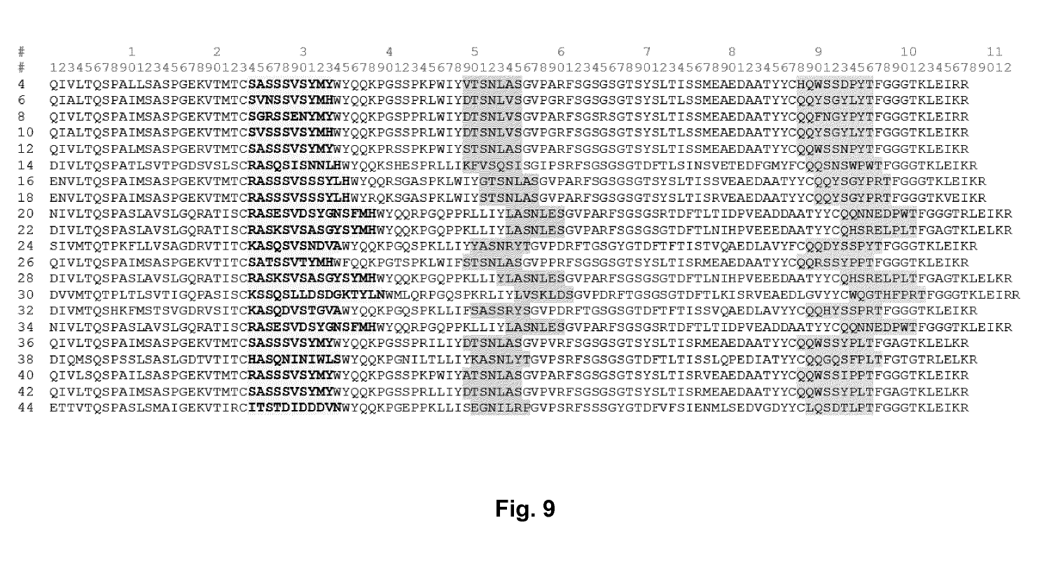

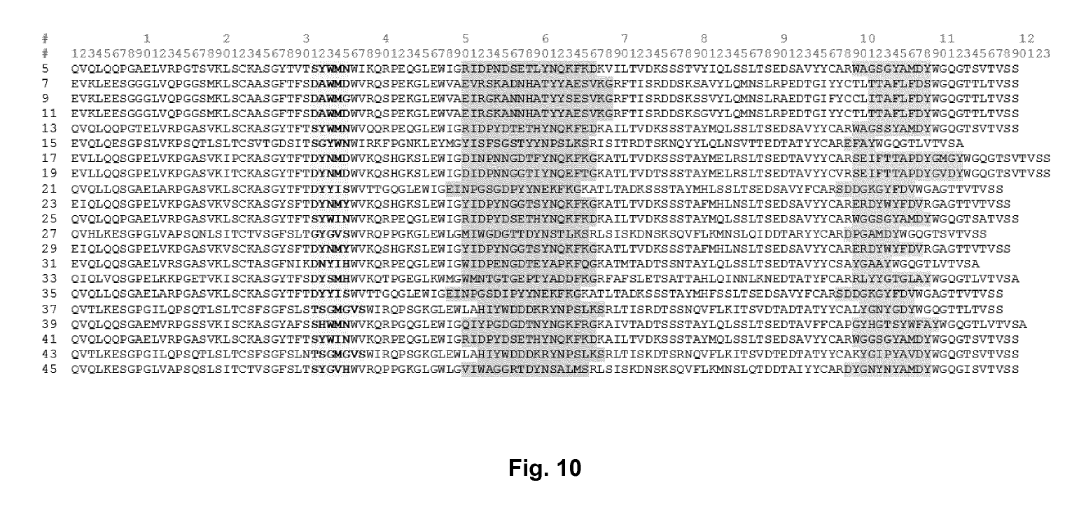

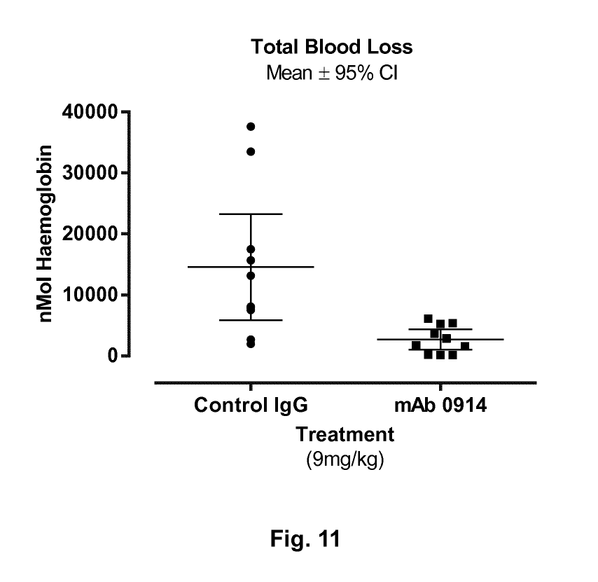

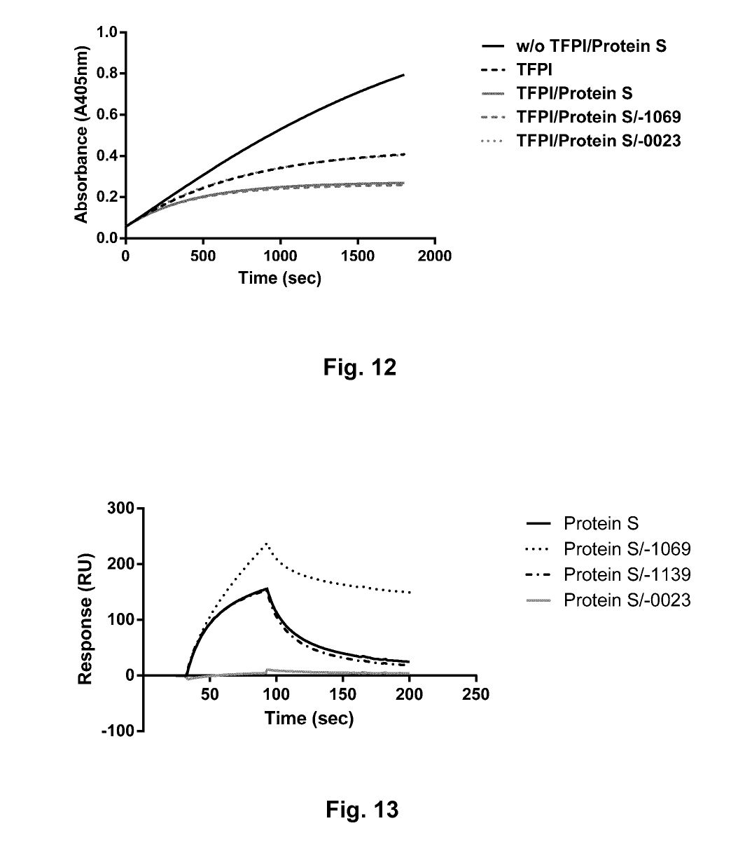

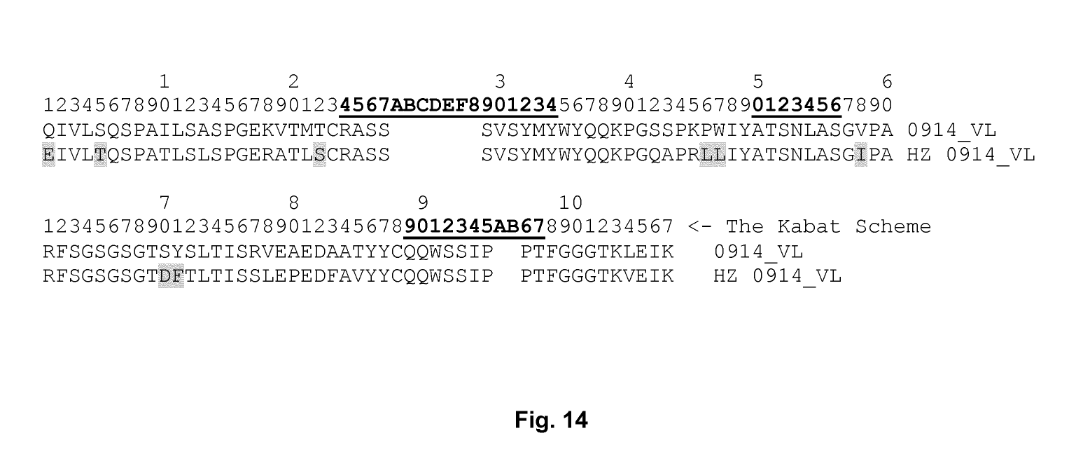

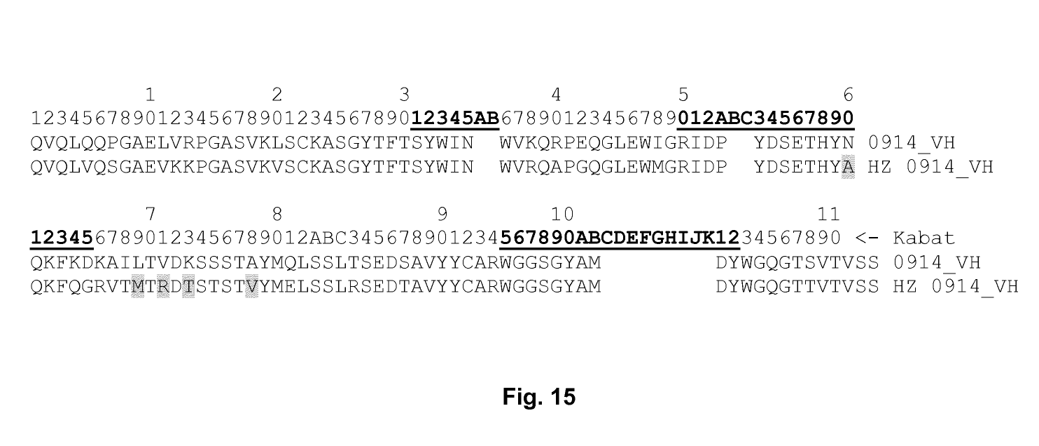

Antibodies increased the thrombin generation in platelet poor severe haemophilia A patient plasma both in the presence and absence of Activated Protein C (APC) in a concentration dependent fashion (FIG. 5). The amount of thrombin generated in plasma was measured by Calibrated Automated Thrombography (Hemker et al. “Calibrated Automated Thrombin Generation Measurement in Clotting Plasma,” Pathophysiol Haemost Thromb. 33:4-15 (2003); Hemker et al. “Thrombin Generation in Plasma: Its Assessment via the Endogenous Thrombin Potential,” Thromb Haemost. 74:134-138 (1995)). In a 96-well plate, 72 μL of Factor VIII deficient plasma pool (<1% residual activity, platelet-poor) from severe haemophilia A patients lacking Factor VIII inhibitor (George King Bio-Medical, Overland Park, Kans.) was incubated with 8 μL of antibody for 10 minutes at 37° C. and then mixed with 10 μL APC or HEPES-BSA buffer and 20 μL Thrombinoscope PPP Trigger (5 pM tissue-factor and 4 μM phospholipid), and reactions were immediately started by mixing with 20 μL fluorogenic substrate (Z-Gly-Gly-Arg-AMC) in HEPES-BSA buffer including 0.1 M CaCl₂. All reagents were pre-warmed to 37° C. The development of a fluorescent signal at 37° C. was monitored at 20 second intervals using a Fluoroskan Ascent reader (Thermo Labsystems OY, Helsinki, Finland). Fluorescent signals were corrected by the reference signal from the thrombin calibrator samples (Hemker et al. “Calibrated Automated Thrombin Generation Measurement in Clotting Plasma,” Pathophysiol Haemost Thromb. 33:4-15 (2003)) and actual thrombin generation in nM was calculated as previously described (Hemker et al. “Thrombin Generation in Plasma: Its Assessment via the Endogenous Thrombin Potential,” Thromb Haemost. 74:134-138 (1995)) (FIG. 5).

### Thrombin Generation in Diluted Rabbit and Cynomolgus Plasma

Antibodies increased the thrombin generation in platelet poor cynomolgus plasma in the presence of thrombomodulin (TM) in a concentration dependent fashion (FIG. 6).The amount of thrombin generated in rabbit and cynomolgus plasma (diluted 1:3 with HEPES-BSA buffer) was measured by Calibrated Automated Thrombography (Hemker et al. “Calibrated Automated Thrombin Generation Measurement in Clotting Plasma,” Pathophysiol Haemost Thromb. 33:4-15 (2003); Hemker et al. “Thrombin Generation in Plasma: Its Assessment via the Endogenous Thrombin Potential,” Thromb Haemost. 74:134-138 (1995)). In a 96-well plate, 72 μL of diluted plasma pool from rabbit or cynomolgus (in house) was incubated with 8 μL of antibody for 10 minutes at 37° C. and then mixed with 10 μL Thrombomodulin (end concentration in plasma 50 nM) (Haematologic Technologies, Inc, VT, USA, HTI Rabbit Thrombomodulin RABT-4202) or HEPES-BSA buffer and 20 μL Thrombinoscope PPP Trigger (5 pM tissue-factor and 4 μM phospholipid), and reactions were immediately started by mixing with 20 μL fluorogenic substrate (Z-Gly-Gly-Arg-AMC) in HEPES-BSA buffer including 0.1 M CaCl₂. All reagents were pre-warmed to 37° C. The development of a fluorescent signal at 37° C. was monitored at 20 second intervals using a Fluoroskan Ascent reader (Thermo Labsystems OY, Helsinki, Finland). Fluorescent signals were corrected by the reference signal from the thrombin calibrator samples (Hemker et al. “Calibrated Automated Thrombin Generation Measurement in Clotting Plasma,” Pathophysiol Haemost Thromb. 33:4-15 (2003)) and actual thrombin generation in nM was calculated as previously described (Hemker et al. “Thrombin Generation in Plasma: Its Assessment via the Endogenous Thrombin Potential,” Thromb Haemost. 74:134-138 (1995)) (FIG. 6).

### Example 14

### Epitope Mapping by HX-MS of Anti-Protein S (EGF1-4) Monoclonal Antibodies

### Introduction to HX-MS

The HX-MS technology utilizes that hydrogen exchange (HX) of a protein, followed by mass spectrometry (MS). By replacing the aqueous solvent containing hydrogen with aqueous solvent containing deuterium, incorporation of a deuterium atom at a given site in a protein will give rise to an increase in mass of 1 Da. This mass increase can be monitored as a function of time by mass spectrometry in quenched samples of the exchange reaction. The deuterium labelling information can be sub-localized to regions in the protein by pepsin digestion under quench conditions and following the mass increase of the resulting peptides.

One use of HX-MS is to probe for sites involved in molecular interactions by identifying regions of reduced hydrogen exchange upon protein-protein complex formation. Usually, binding interfaces will be revealed by marked reductions in hydrogen exchange due to steric exclusion of solvent. Protein-protein complex formation may be detected by HX-MS simply by measuring the total amount of deuterium incorporated in either protein members in the presence and absence of the respective binding partner as a function of time. The HX-MS technique uses the native components, i.e., protein and antibody or Fab fragment, and is performed in solution. Thus HX-MS provides the possibility for mimicking the in vivo conditions (for a review on the HX-MS technology, see e.g. Wales and Engen, Mass Spectrom. Rev. 25, 158 (2006)).

### Materials

### Proteins Used:

Human Protein S: The protein molecule containing all EGF1-4 domains of human Protein S (SEQ ID NO: 2) fused to a C-terminal HCP4 purification tag was used in the present study (EGF1-4).

### mAb Molecules:

0322-0000-0017
0322-0000-0114
0322-0000-0158
0322-0000-0203
0322-0000-1069 (murine-human IgG4 chimer)
All proteins were buffer exchanged into 25 mM MES pH 6.5, 5 mM CaCl₂, 150 mM NaCl before experiments.

### Methods: HX-MS Experiments

### Instrumentation and Data Recording

The HX experiments were performed on a nanoACQUITY UPLC System with HDX Technology (Waters Inc.) coupled to a Synapt G2 mass spectrometer (Waters Inc.). The Waters HDX system contained a Leap robot (H/D-x PAL; Waters Inc.) operated by the LeapShell software (Leap Technologies Inc/Waters Inc.), which performed initiation of the deuterium exchange reaction, reaction time control, quench reaction, injection onto the UPLC system and digestion time control. The Leap robot was equipped with two temperature controlled stacks maintained at 20° C. for buffer storage and HX reactions and maintained at 2° C. for storage of protein and quench solution, respectively. The Waters HDX system furthermore contained a temperature controlled chamber holding the pre- and analytical columns, and the LC tubing and switching valves at 0.5° C. A separately temperature controlled chamber holds the pepsin column at 25° C. For the inline pepsin digestion, 100 μL quenched sample containing 200 pmol EGF1-4 was incubated for 60 sec at 2° C. and then injected and passed over a Poroszyme® Immobilized Pepsin Cartridge (2.1×30 mm (Applied Biosystems)) placed at 25° C. using a isocratic flow rate of 100 μL/min (0.1% formic acid:CH₃CN 95:5). The resulting peptides were trapped and desalted on a VanGuard pre-column BEH C18 1.7 μm (2.1×5 mm (Waters Inc.)). Subsequently, the valves were switched to place the pre-column inline with the analytical column, UPLC-BEH C18 1.7 μm (1×100 mm (Waters Inc.)), and the peptides separated using a 9 min gradient of 10-50% B delivered at 40 μl/min from the nanoAQUITY UPLC system (Waters Inc.). The mobile phases consisted of A: 0.1% formic acid and B: 0.1% formic acid in CH₃CN. The ESI MS data, and the separate elevated energy (MS^{E}) experiments were acquired in positive ion mode using a Synapt G2 mass spectrometer with ion mobility (Waters Inc.). Leucine-enkephalin was used as the lock mass ([M+H]⁺ ion at m/z 556.2771) and data was collected in continuum mode (For further description, see Andersen and Faber, Int. J. Mass Spec., 302, 139-148 (2011)).

### Data Analysis

Peptic peptides were identified in separate experiments using standard MS^{E} methods where the peptides and fragments are further aligned utilizing the ion mobility properties of the Synapt G2 (Waters Inc.). MS^{E} data were processed using Protein Lynx Global Server version version 2.5 (Waters Inc.) and optional hydroxylation of Asn or Asp was included in the peptide searches since the EGF domains contains this post-translational modification. The HX-MS raw data files were processed in the DynamX 2.0 software (Waters Inc.). DynamX automatically performs the lock mass-correction and deuterium incorporation determination, i.e., centroid determination of deuterated peptides. Furthermore, all peptides were inspected manually to ensure correct peak and deuteration assignment by the software.

### Epitope Mapping Experiment

Amide hydrogen/deuterium exchange (HX) was initiated by a 10-fold dilution of EGF1-4 in the presence or absence of mAb 0322-0000-0017, 0322-0000-0114, 0322-0000-0158, 0322-0000-0203 or 0322-0000-1069 into the corresponding deuterated buffer (i.e., 25 mM MES, 5 mM CaCl₂, 150 mM NaCl prepared in D₂O from concentrated stocks, 94% D₂O final, pH 6.5 (uncorrected value)). All HX reactions were carried out at 20° C. and contained 4 μM EGF1-4 in the absence or presence of 2.4 μM mAb thus giving a 1.2 fold molar excess of mAb binding regions. At time intervals 0.25, 0.5, 1, 3, 10 and 30 minutes, 50 μl aliquots of the HX reaction were quenched by 50 μl ice-cold quenching buffer (1.35 M TCEP, 2 M Urea) resulting in a final pH of 2.5 (uncorrected value).

### Results and Discussion

### Human Protein S Protein

A protein molecule containing the EGF1-4 of human Protein S domains was used for the present study. The EGF domains contain hydroxylation of an Asn or Asp residue involved in Ca²⁺ binding (Stenberg et al. J. Biol. Chem. (1997) 272:23255-23260). Both unmodified sequence and hydroxylated sequence was detected in the MS-MS experiments and both versions of these peptic peptides were included in the data analysis (cf. table 4). All numbering in this example and table 4 refers to SEQ ID NO: 2.

### HX-MS Analysis

The HX time-course of 42 peptic peptides, covering 95% of the primary structure of EGF1-4 was monitored in the absence or presence of mAb 0322-0000-0017, 0322-0000-0114, 0322-0000-0158, 0322-0000-0203 or 0322-0000-1069. The observed exchange pattern in the early time-points (<10 min) in the presence or absence of mAb 0322-0000-0017, 0322-0000-0114, 0322-0000-0158, 0322-0000-0203 or 0322-0000-1069 can be divided into two different groups: One group of EGF1-4 peptic peptides display an exchange pattern that is unaffected by the binding of mAbs. In contrast, another group of peptides in EGF1-4 show protection from exchange upon mAb binding (cf. table 4). In the case of overlapping peptic peptides, the exchange protection information is attempted sub-localized to specific stretches within the peptide assuming full back-exchange of the peptide N-terminus and first peptide bond. Exchange protection in a peptide is indicative of this region being involved in mAb binding. Thus the epitope is partly or fully located within the region defined by the specific peptides. However, since the resolution of HX-MS is based on pepsin digestion of the deuterated protein, exchange protection within a given region does not imply that every residue within the region defined by the peptic peptides necessarily is involved in mAb binding.

### Epitope Mapping of mAb 0322-0000-0017, 0322-0000-0203 and 0322-0000-1069

The HX pattern of mAb 0322-0000-0017, mAb 0322-0000-0203 and mAb 0322-0000-1069 was similar and will therefore be described combined here. Epitope signal for mAb 0322-0000-0017, -0203 and -1069 were observed in the EGF1 domain up until residue Phe43 (cf. table 4). The exchange protection became stronger, the longer the peptides extended from the starting points (residues 1 or 4) thus indicating exchange protection in the more C-terminal region of the peptides. In contrast the peptides 1-15, 4-15 and 4-19 did not show exchange protection.

Therefore the epitope for 0322-0000-0017, 0322-0000-0203 and for 0322-0000-1069 arises from the SCKDGKASFTCTCKPGWQGEKCEF sequence within the EGF1 domain i.e., residues 20-43 of SEQ ID NO: 2.

### Epitope Mapping of mAb 0322-0000-0114 and 0322-0000-0158

The HX pattern of mAb 0322-0000-0114 and mAb 0322-0000-0158 was similar and will therefore be described combined here. Epitope signal for mAb 0322-0000-0114 and -0158 were observed in the EGF2 domain in peptides starting at residue Va178 (cf. table 4). The exchange protection continued into the EGF3 domain and in peptides up until residue Phe111. However, since no exchange protection is observed in peptides staring at residue 103 and higher (cf. table 4), residues 105 and higher can be excluded from the epitope region. Therefore the epitope for both 0322-0000-0114 and for 0322-0000-0158 arises from the VMLSNKKDCKDVDECSLKPSICGTAVCK sequence within the EGF2-3 domain i.e., residues 78-105 of SEQ ID NO: 2.

TABLE 4
HX-MS analysis of human Protein S EGF1-4
HX-MS analysis of human Protein S EGF1-4 yielding epitope information for antibody molecules
0322-0000-0017, 0322-0000-0114, 0322-0000-0158, 0322-0000-0203 and 0322-0000-1069. After
deuterium exchange reaction, EGF1-4 was digested with pepsin yielding the following peptic
peptide regions that were analyzed. Numbering of EGF1-4 residues in this table follows SEQ ID
NO: 2, however, the C-terminal HCP4 tag A174- K188 is not part of the natural human Protein
S sequence.

| Sequence | Modification | Domain | 0017 | 0114 | 0158 | 0203 | 1069 |
|---|---|---|---|---|---|---|---|
| V1-E15 | | EGF1 | N | na | N | N | N |
| V1-F28 | | EGF1 | EX | N | N | EX | EX |
| V1-F28 | hydroxylated | EGF1 | EX | N | N | EX | EX |
| V1-F43 | | EGF1 | EX | N | N | EX | EX |
| V1-F43 | hydroxylated | EGF1 | EX | N | N | EX | EX |
| 14-E15 | | EGF1 | N | N | N | N | N |
| 14-M19 | | EGF1 | N | N | na | N | N |
| 14-F28 | | EGF1 | EX | N | N | EX | EX |
| 14-F28 | hydroxylated | EGF1 | EX | N | N | EX | EX |
| 14-F43 | | EGF1 | EX | N | N | EX | EX |
| 14-F43 | hydroxylated | EGF1 | EX | N | N | EX | EX |
| C48-F77 | | EGF2 | N | N | N | N | N |
| V78-C99 | | EGF2/EGF3 | N | EX | EX | N | N |
| V78-T101 | | EGF2/EGF3 | N | EX | EX | N | na |
| V78-A102 | | EGF2/EGF3 | na | EX | EX | N | N |
| V78-F111 | | EGF2/EGF3 | N | EX | EX | N | N |
| V78-F111 | hydroxylated | EGF2/EGF3 | N | EX | EX | N | N |
| C92-T101 | | EGF3 | N | EX | EX | N | N |
| C92-A102 | | EGF3 | N | EX | EX | N | N |
| C92-C104 | | EGF3 | na | EX | EX | N | N |
| C92-F111 | | EGF3 | N | EX | EX | N | N |
| C92-F111 | hydroxylated | EGF3 | N | EX | EX | N | na |
| G100-F111 | | EGF3 | N | EX | EX | N | N |
| A102-F111 | | EGF3 | N | EX | EX | N | N |
| V103-F111 | | EGF3 | N | N | N | N | N |
| E112-L123 | | EGF3 | N | N | na | na | N |
| E112-E133 | | EGF3/EGF4 | N | N | N | N | N |
| C139-E170 | hydroxylated | EGF4 | N | N | N | N | N |
| L142-E170 | | EGF4 | na | N | N | N | na |
| L142-E170 | hydroxylated | EGF4 | N | N | N | N | N |
| C143-E170 | | EGF4 | N | N | N | N | N |
| C143-E170 | hydroxylated | EGF4 | N | N | N | N | N |
| C143-L175 | hydroxylated | EGF4 | N | N | N | N | N |
| N145-E170 | hydroxylated | EGF4 | N | N | N | N | N |
| Y153-E170 | | EGF4 | N | N | N | N | na |
| V171-L184 | | EGF4/HCP4 | N | N | N | N | N |
| V171-G187 | | EGF4/HCP4 | N | N | N | N | N |
| L175-L184 | | HCP4 | N | N | N | N | N |
| L175-G187 | | HCP4 | N | N | N | N | N |
| A176-L184 | | HCP4 | N | N | N | N | N |
| D178-L184 | | HCP4 | N | N | N | N | N |
| D178-G187 | | HCP4 | N | na | N | N | N |

EX: exchange protection upon antibody binding indicating epitope region (>0.3 Da on at least three time-points).
N: No exchange protection upon antibody binding (<0.3 Da).
na: Not analyzable in respective experiment.

### Example 15

### Residue Specific Epitope Mapping by HX-MS of Anti-Protein S (EGF1-4) Monoclonal Antibody

This example further describes the epitope of the antibody NNC 0322-0000-1069 mapped on the Protein S EGF1-4 domain (SEQ ID NO: 2). It is an extension of the epitope mapping experiments described in Example 14. The experiments described here are based on the same principle of hydrogen-deuterium exchange as the experiments in Example 14, but further utilizes fragmentation of the peptides to enable residue-specific determination of deuterium incorporation.

Epitope mapping with resolution down to the single residue level was conducted using Hydrogen-Deuterium Exchange (HX) mass spectrometry (MS) combined with electron transfer dissociation (ETD) fragmentation of hydrogen-deuterium exchanged peptides.

ETD causes fast fragmentation of the peptide while retaining the positions of hydrogen-deuterium exchanged protons on the backbone amide nitrogens. This way it is possible to map deuterium incorporation down to single residues in the protein backbone.

Fragmentation by ETD breaks the backbone of the peptide between the amide nitrogen and the C-alpha carbon. The fragment containing the N-terminal part of the peptide is denoted the C-fragment and the fragment containing the C-terminal part of the peptide is denoted the Z-fragment. The C1-fragment will consist of the first (N-terminal) residue of the peptide as well as the backbone amide of the second residue of the peptide. Likewise, the Z1 fragment consists of the last (C-terminal) residue of the peptide apart from the backbone amide group. The C-fragments and the corresponding residues from which backbone amide HX can be determined are listed in table 5 and the Z-fragments and the corresponding residues from which backbone amide HX can be determined are listed in table 6 in the results section below.

### Experiments

Solutions of Protein S EGF1-4 alone or in the presence of one of the antibodies antibody 0322-0000-1069 were diluted 25-fold in deuterated MES buffer (25 mM MES, 150 mM sodium chloride, 5 mM calcium chloride, pH 6.5). Non-deuterated controls were prepared by diluting into protiated MES buffer. The hydrogen exchange experiments were performed on a nanoAcquity UPLC system with HDX technology (Waters Corporation, Milford, Mass., USA) which includes the HD-x PAL auto sampler (LEAP Technologies Inc., Carrboro, N.C., USA) for automated sample preparation and an ultra-high performance liquid chromatography (UPLC) system. The UPLC tubing, pre- and analytical columns and switching valves were located in a chamber cooled to 0.3° C. The trypsin digestion column was stored at 25° C. Hydrogen exchange reactions were performed at 20° C. Mass analysis was performed online using a Waters SYNAPT G2 HDMS mass spectrometer.

A volume containing 300 pmol of Protein S EGF1-4 with or without 330 pmol of the antibody was diluted into deuterated MES buffer. At the time intervals 15 seconds, minute, 4 minutes, and 16 minutes. 50 μl of the sample was quenched in 50 μl 1.35 mM Tris (2-carboxyethyl) phosphine adjusted to pH 2.7 and held at 3° C. The quenched sample was incubated at 3° C. for 60 seconds and 99 μl of the quenched solution was then immediately injected and passed over a Porozyme immobilized pepsin column (2.1 mm×30 mm) (Applied Biosystems, Life Technologies Corporation, Carlsbad, Calif., USA) and trapped on a Waters VanGuard BEH C18 1.7 μm (2.1 mm×5 mm) column using a 5% methanol, 0.1% formic acid mobile phase and a 100 μl/min flow rate. The peptides were separated on a Waters UPLC BEH C18 1.7 μm (1.0 mm×100 mm) column using a 15 min 10-40% acetonitrile gradient containing 0.1% formic acid at a 40 μl/min flow-rate. The mobile phases were added 0.1% 3-nitrobenzyl alcohol for supercharging to enhance ETD fragmentation.

The mass spectrometer was run in positive ion mode with ETD fragmentation enabled. The instrument parameters used were 3.0 kV capillary, 18 V sample cone, and 4 V extraction cone offsets, 100 ml/min flow of desolvation gas and 25 ml/min cone gas flow. The source block was heated to 90° C. and the desolvation gas to 350° C. The trap and transfer regions were flushed with a 14 ml/min buffer gas flow to trap the ions. The trap wave height was lowered to 0.5 V for efficient ETD fragmentation. 1,4-di-cyano benzene was used as ETD reagent and ions were created using 25 ml/min MakeUp gas flow and 71 V discharge current. Based on the results of the epitope mapping described in Example 14, the peptide D16-F28 and T29-F43 were selected for residue-specific epitope mapping as these peptides covered the epitope of NNC 0322-0000-1069 on Protein S EGF1-4 were abundant and resulted in high

The data was analysed manually using an in-house macro for Microsoft Excel, which determines the mean mass of a specified interval by taking the average of the m/z values weighed by the intensity. Exchange protection was calculated by subtracting the mean mass of the fragment measured in presence of mAb 0322-0000-1069 from the mean mass of the fragment measured in the absence of antibody. The degree of protection was determined as the average of the four incubation times included in the experiment. 2 replicates were measured of each sample and the results were averaged.

### Results

The results from the peptide D16-F28 were inconclusive. The protection from hydrogen-deuterium exchange upon binding of the antibody 0322-0000-1069 to the Protein S EGF1-4 domains are shown in table 5 (C-ion fragment series) and table 6 (Z-ion fragment series), respectively.

TABLE 5
Peptide T29-F43 ETD C-ion fragments
and site specific hydrogen
exchange protection

| | | Average |
|---|---|---|
| Frag- | | exchange |
| ment | Sequence | protection |
| C1 | TC | N/A |
| C2 | TCT | N/A |
| C3 | TCTC | N/A |
| C4 | TCTCK | N/A |
| C5 | TCTCKP | N/A |
| C6 | TCTCKPG | 0.04 Da |
| C7 | TCTCKPGW | 0.11 Da |
| C8 | TCTCKPGWQ | 0.19 Da |
| C9 | TCTCKPGWQG | 0.13 Da |
| C10 | TCTCKPGWQGE | N/A |
| C11 | TCTCKPGWQGEK | 0.52 Da |
| C12 | TCTCKPGWQGEKC | N/A |
| C13 | TCTCKPGWQGEKCE | 0.71 Da |
| C14 | TCTCKPGWQGEKCEF | N/A |

Increase in exchange protection is considered
significant when above 0.09 Da

Deuterium incorporation for Fragments C1-05 could not be determined. An exchange protection of 0.04 Da was observed for the C6 fragment. This was not considered a significant difference as it is below 0.09 Da. A significant increase in exchange protection was observed for fragment C7 indicating that residue W36 is involved in antibody binding. No significant increase in exchange protection was observed for fragments C8 and C9. Deuterium incorporation for fragment C10 could not be determined. A significant increase in hydrogen exchange protection was observed for fragment C11 indicating that at least one of the residues E39 and K40 are involved in antibody binding. The large increase in hydrogen exchange protection (0.39 Da) could support that both residues are contributing to antibody binding. Deuterium incorporation for fragment C12 could not be determined. A significant increase in exchange protection was observed for fragment C13 indicating that one or both of the residues C41 and E42 are contributing to antibody binding.

TABLE 6
Peptide T29-F43 ETD Z-ion fragments
and site specific hydrogen
exchange protection

| | | Average |
|---|---|---|
| Frag- | | exchange |
| ment | Sequence | protection |
| Z1 | N/A | N/A |
| Z2 | F | N/A |
| Z3 | EF | N/A |
| Z4 | CEF | 0.09 |
| Z5 | KCEF | 0.18 |
| Z6 | EKCEF | N/A |
| Z7 | GEKCEF | 0.34 |
| Z8 | QGEKCEF | 0.34 |
| Z9 | WQGEKCEF | N/A |
| Z10 | GWQGEKCEF | 0.46 |
| Z11 | PGWQGEKCEF | 0.38 |
| Z12 | KPGWQGEKCEF | 0.47 |
| Z13 | CKPGWQGEKCEF | 0.49 |
| Z14 | TCKPGWQGEKCEF | N/A |

Increase in exchange protection is considered
significant when above 0.09 Da

Deuterium incorporation for fragments Z1-Z3 could not be determined. A significant hydrogen exchange protection was observed for fragment Z4 indicating that one or more of the residues C41, E42, and F43 are contributing to antibody binding. A significant increase in hydrogen exchange protection was observed for the Z5 fragment indicating that residue K40 is contributing to antibody binding. Deuterium incorporation for fragment Z6 could not be determined. An increase in hydrogen exchange protection was observed for the Z7 fragment indicating that one or both of the residues G38 and E39 are contributing to antibody binding. No significant increase in hydrogen exchange protection was observed for the Z8 fragment. Deuterium incorporation for the Z9 fragment could not be determined. A significant increase in hydrogen exchange protection was observed for the Z10 fragment indicating that one or both of the residues G35 and W36 are contributing to antibody binding. No significant increase in hydrogen exchange protection was observed for the fragments Z11, Z12, or Z13. Deuterium incorporation for fragment Z14 could not be determined.

The C-ion fragment series revealed that when binding the antibody NNC 0322-0000-1069 hydrogen deuterium exchange protection is observed for the residues W36, one or both of the residues E39 and K40, and one or both of the residues C41, and E42. In addition the Z-ion fragment series revealed that the one or both of the residues G35 and W36, one or both of the residues G38 and E39, residue K40, and one or more of the residues C41, E42, and F43 are protected from hydrogen deuterium exchange upon antibody binding.

Combining the information gained from the C-ion and Z-ion fragment series thus limits the residues protected from hydrogen-deuterium exchange upon antibody binding to the residues W36, E39, K40, and one or more of the residues C41, E42 and F43.

Thus, the residues protected from hydrogen-deuterium exchange upon binding of the antibody NNC 0322-0000-1069 to Protein S (EGF1-4) include the residues W36, E39, K40, and one or more of the residues C41, E42 and F43.

### Example 16

### Interaction of Protein S/Anti-Protein S Antibody Complexes with Lipid Surfaces

The binding of human Protein S/anti-Protein S complexes to phosphatidylserine-containing lipid vesicles was evaluated by surface plasmon resonance using the Biacore3000 instrument. Lipid vesicles were captured on a L1 sensor chip (GE healthcare cat# BR-1005-58) as described in Hodnik et al. Methods Mol Biol. (2010) 627: 201-11.

Phosphatidylserine-containing lipid vesicles (Avanti Polar Lipids, Inc.; cat#211635) were immobilized on the active flow cell and phosphocholine vesicles (Avanti Polar Lipids, Inc.; cat#211621) were immobilized on the reference cell.

Human Protein S (Haematologic Technologies Inc.; cat# HCPS-0090) (100 nM) was captured on the lipid surface (approximately 200 RU) and the binding of monoclonal antibodies (250 nM) was monitored. Bound proteins were removed from the sensor surfaces by EDTA-containing regeneration buffer.

FIG. 7 shows surface plasmon resonance (SPR) sensorgrams for binding of monoclonal antibodies 0322-0000-0114 (solid line) and 0322-0000-0203 (dotted line) to Protein S captured on phosphatidylserine-containing lipid vesicles. The antibodies are able to bind Protein S bound on a lipid surface.

In a similar experiment, serial dilution of Protein S (100 nM and 2-fold dilution series) was incubated with saturating concentration of monoclonal antibodies (500 nM) prior to injection over the chip surface. From the binding sensorgrams an estimated affinity for binding of Protein S/anti-Protein S complexes to the lipid surface was derived and compared to free Protein S. Affinities were as follows: Free Protein S: 2.5 nM, Protein S/0114: 4.0 nM, Protein S/0203: 4.0 nM.

FIG. 8 shows SPR sensorgrams for binding of free Protein S (100 nM) or Protein S (100 nM) incubated with a specified monoclonal antibody (500 nM) to phosphatidylserine-containing lipid vesicles. The antibody 2F140, an antibody binding the Gla-domain of Protein S, was included as a control. 2F140 prevents, as expected, the binding of Protein S to the lipid surface.

It can be concluded that the binding affinity of Protein S to the lipid surface was retained in the presence of 0322-0000-0114 and -0203 and hence that said monoclonal antibodies do not prevent Protein S binding to the lipid surface.

### Example 17

### The In Vivo Effect of Anti-Protein S mAb 0914

The in vivo effect of an anti-Protein S antibody on cuticle bleeding was examined in a rabbit model of induced haemophilia A as described by Hilden et al. Blood (2012) Jun 14; 119(24):5871-8. Briefly, anaesthetized rabbits were made transiently haemophilic by intravenous administration of a monoclonal anti human FVIII antibody with cross-reactivity to rabbit FVIII (2000 rabbit Bethesda units per kg). Eight minutes after anti FVIII administration, two groups of 10 rabbits were dosed with either anti-Protein S antibody 0322-0000-0914 (mAb 0914) or an isotype control antibody at 9 mg/kg using a dosing volume of 1.18 ml/kg. After another 12 minutes, bleeding was induced by cutting the tip of the nail of the third digit, including the apex of the cuticle, and blood was collected in 37° C. saline for 60 minutes thereafter. Bleeding was quantified by measurement of haemoglobin bled into the saline.

The anti-Protein S antibody caused a statistically significant (p=0.013) reduction in mean blood loss from 14,563 nmol of haemoglobin (95% CI: 5,845-23,281 nmol) to 2,712 nmol (95% CI: 1,060-4,363 nmol) as determined by two-tailed T-test with Welch's correction for different variances, see table 7 below and FIG. 11.

TABLE 7
Average blood loss in rabbits transiently induced with haemophilia
A and treated with anti-Protein S mAb 0914 or an isotype control

| | Isotype control antibody | mAb 0914 |
|---|---|---|
| Average blood loss | 14,563 | 2,712 |
| (nmol haemoglobin) | (95% CI: 5,845-23,281) | (95% CI: 1,060-4,363) |

### Example 18

### Interaction of Protein S/Protein S-mAb Complexes with C4b-Binding Protein

The binding of free human Protein S (from Enzyme Research Laboratories, Cat# HPS) and protein S in complex with mAb 0322-0000-1069 or the corresponding Fab fragment (0322-0000-1139) to C4b-Binding Protein (C4BP; from Hyphen BioMed, cat# PP015A) was evaluated by surface plasmon resonance using the Biacore T200 instrument.

In brief, a polyclonal anti-C4BP antibody targeting the alpha chain (ab83755 from abcam) was immobilized on a CM5 Biacore sensor chip by standard amine coupling chemistry. C4BP was captured followed by injection of serial dilutions of free Protein S or Protein S incubated with molar excess of mAb/Fab. The experiment was conducted in 10 mM Hepes, pH 7.4; 150 mM NaCl; 10 M CaCl₂ supplemented with 0.005% Tween20 and the chip was regenerated with glycine-HCl pH 1.7. The estimated affinity for binding of free protein S to C4BP was determined to 2 nM. As a control, binding of Protein S in complex with a LamG-specific antibody 0322-0000-0023 was investigated. The binding of C4BP and Protein S is mediated through the LamG domains of Protein S (He X. et al. Biochemistry, 1997; 36(12): 3745-54) and as anticipated 0322-0000-0023 completely blocks the interaction between Protein S and C4BP.

Neither the mAb nor the Fab prevents binding of Protein S to C4BP. To further support the conclusion that mAb and Fab do not interfere with Protein S binding to C4BP the affinity of the 0322-0000-1139 to Protein S captured on C4BP was determined. 0322-0000-1139 binds to protein S immobilized on C4BP with an affinity resembling the affinity of protein S binding to the full length antibody 0322-0000-1069 (K_{D} of 10 and 20 nM, respectively).

### Example 19

### Stimulation of Thrombelastography in Haemophilia A-Like Blood

The elastic properties of blood during thrombus formation were measured via thrombelastography using a TEG® hemostasis analyzer (U.S. Pat. No. 5,223,227, and Luddington, R J, “Thrombelastography/thromboelastometry” Clin Lab Haematol. 27:81-90 (2005)). The TEG® hemostasis analyzer monitors the elastic properties of blood as it is induced to clot under a low shear environment resembling sluggish venous blood flow. The patterns of changes in shear elasticity of the developing clot enable the determination of the kinetics of clot formation, as well as the strength and stability of the formed clot; in short, the mechanical properties of the developing clot. In a TEG® a total volume of 340 μL of pre-heated (37° C.) human whole blood is incubated with combinations of compound, neutralising polyclonal anti-FVIII antibody, activated protein C (APC) or thrombomodulin (TM) and tissue factor (TF). This blood is recalcified with 20 μL calcium chloride (0.2 M), initiating TEG analysis.

The R-value (s) is the clot time, defined as the time from initiation to when the amplitude reaches 2 mm. Maximum rate of thrombus generation (MTG; mm×100/s) is defined as the global maximum of the first derivative of amplitude in time.

### Results

Tables 8 through 11 provide the thrombelastography parameters R-value and MTG determined in normal and haemophilia A-like human blood (i.e., in the absence or presence of a neutralising anti-FVIII polyclonal antibody, 0.1 mg/mL). Tables 8 and 9 show these values in the presence of 1 nM APC and increasing concentrations of antibodies 0322-0000-0114, 0322-0000-0910 and 0322-0000-0914 (0 nM to 1633 nM) (table 8 for R-value and table 9 for MTG).

Tables 10 and 11 show these values in the presence of 5 nM TM and increasing concentrations of the same antibodies (table 10 for R-value and table 11 for MTG). Each antibody was tested in blood from two different donors. Thrombelastography was initiated using 40,000-fold diluted TF (Innovin, approximately 6 nM stock solution, 150 fM end-concentration). All three Protein S antibodies concentration-dependently reduce the R-value and increase MTG.

TABLE 8
R-value in normal or haemophilia A-like whole blood, with 0 nM to 1633 nM
Protein S antibody and 1 nM APC

| | | Protein S | R-value (s) | | | | | |
|---|---|---|---|---|---|---|---|---|
| | | antibody | 0322-0000-0114 | | 0322-0000-0910 | | 0322-0000-0914 | |
| FVIII | | conc. | Donor | Donor | Donor | Donor | Donor | Donor |
| antibody | APC | (nM) | 1 | 2 | 3 | 4 | 5 | 6 |
| No | No | 0 | 12.6 | 8.0 | 11.8 | 9.8 | 10.9 | 12.3 |
| Yes | No | 0 | 24.7 | 17.1 | 20.4 | 13.5 | 41.3 | 27.3 |
| Yes | Yes | 0 | 33.3 | 32.2 | 40.8 | 19.8 | 87.5 | 58.5 |
| Yes | Yes | 1 | 32.1 | 43.2 | 50.5 | 27.4 | 75.7 | 57.1 |
| Yes | Yes | 3 | 34.7 | 44.2 | 41.3 | 17.3 | 79.3 | 64.2 |
| Yes | Yes | 10 | 35.8 | 29.1 | 24.8 | 15.8 | 55.6 | 51.3 |
| Yes | Yes | 30 | 30.6 | 21.2 | 27.4 | 16.8 | 41.0 | 28.5 |
| Yes | Yes | 100 | 28.8 | 15.4 | 24.1 | 14.4 | 19.6 | 18.8 |
| Yes | Yes | 300 | 17.4 | 10.8 | 19.6 | 9.9 | 20.4 | 14.5 |
| Yes | Yes | 1000 | 13.6 | 13.2 | 11.6 | 12.8 | 14.3 | 13.5 |
| Yes | Yes | 1189 | N.D.* | N.D. | N.D. | N.D. | 20.8 | 11.8 |
| Yes | Yes | 1633 | N.D. | N.D. | 12.8 | 11.3 | N.D. | N.D. |

*N.D. indicates not determined

TABLE 9
MTG in normal or haemophilia A-like whole blood, with 0 nM to 1633 nM
Protein S antibody and 1 nM APC

| | | Protein S | MTG (mmx100/s) | | | | | |
|---|---|---|---|---|---|---|---|---|
| | | antibody | 0322-0000-0114 | | 0322-0000-0910 | | 0322-0000-0914 | |
| FVIII | | conc. | Donor | Donor | Donor | Donor | Donor | Donor |
| antibody | APC | (nM) | 1 | 2 | 3 | 4 | 5 | 6 |
| No | No | 0 | 5.2 | 7.9 | 9.1 | 9.1 | 7.3 | 7.0 |
| Yes | No | 0 | 2.6 | 3.4 | 2.8 | 4.7 | 1.8 | 2.5 |
| Yes | Yes | 0 | 1.5 | 3.5 | 1.3 | 4.1 | 0.9 | 1.6 |
| Yes | Yes | 1 | 1.2 | 1.6 | 0.0 | 3.0 | 1.6 | 9.8 |
| Yes | Yes | 3 | 1.4 | 2.3 | 0.0 | 3.0 | 4.2 | 7.3 |
| Yes | Yes | 10 | 1.2 | 2.3 | 2.1 | 4.2 | 2.1 | 1.2 |
| Yes | Yes | 30 | 2.1 | 2.6 | 2.6 | 3.8 | 1.9 | 2.1 |
| Yes | Yes | 100 | 1.6 | 3.5 | 1.6 | 5.4 | 2.3 | 3.3 |
| Yes | Yes | 300 | 2.3 | 5.6 | 1.6 | 6.3 | 3.0 | 4.7 |
| Yes | Yes | 1000 | 4.0 | 4.5 | 4.2 | 4.2 | 4.9 | 5.4 |
| Yes | Yes | 1189 | N.D. | N.D. | N.D. | N.D. | 2.8 | 9.6 |
| Yes | Yes | 1633 | N.D. | N.D. | 4.9 | 9.4 | N.D. | N.D. |

*N.D. indicates not determined

TABLE 10
R-value in normal or haemophilia A-like whole blood, with 0 nM to 1633 nM
Protein S antibody and 5 nM TM

| | | Protein S | R-value (s) | | | | | |
|---|---|---|---|---|---|---|---|---|
| | | antibody | 0322-0000-0114 | | 0322-0000-0910 | | 0322-0000-0914 | |
| FVIII | | conc. | Donor | Donor | Donor | Donor | Donor | Donor |
| antibody | TM | (nM) | 1 | 2 | 3 | 4 | 5 | 6 |
| No | No | 0 | 12.6 | 8.0 | 11.8 | 9.8 | 10.9 | 12.3 |
| Yes | No | 0 | 24.7 | 17.1 | 20.4 | 13.5 | 41.3 | 27.3 |
| Yes | Yes | 0 | 86.4 | 56.0 | 80.3 | 43.5 | 144.0 | 90.7 |
| Yes | Yes | 1 | 76.8 | 64.2 | 42.8 | 38.4 | 155.4 | 96.5 |
| Yes | Yes | 3 | 69.1 | 66.4 | 62.6 | 27.2 | 121.3 | 63.8 |
| Yes | Yes | 10 | 103.6 | 49.6 | 61.8 | 45.3 | 125.5 | 83.5 |
| Yes | Yes | 30 | 79.9 | 51.3 | 62.5 | 27.9 | 112.8 | 58.5 |
| Yes | Yes | 100 | 37.0 | 35.8 | 65.3 | 30.9 | 48.7 | 40.2 |
| Yes | Yes | 300 | 29.3 | 23.6 | 48.0 | 18.3 | 31.7 | 23.0 |
| Yes | Yes | 1000 | 22.9 | 0.0 | 23.8 | 19.3 | 26.1 | 17.6 |
| Yes | Yes | 1189 | N.D. | N.D. | N.D. | N.D. | 32.5 | 18.2 |
| Yes | Yes | 1633 | N.D. | N.D. | 22.5 | 17.0 | N.D. | N.D. |

*N.D. indicates not determined

TABLE 11
MTG in normal or haemophilia A-like whole blood, with 0 nM to 1633 nM
Protein S antibody and 5 nM TM

| | | Protein S | MTG (mmx100/s) | | | | | |
|---|---|---|---|---|---|---|---|---|
| | | antibody | 0322-0000-0114 | | 0322-0000-0910 | | 0322-0000-0914 | |
| FVIII | | conc. | Donor | Donor | Donor | Donor | Donor | Donor |
| antibody | TM | (nM) | 1 | 2 | 3 | 4 | 5 | 6 |
| No | No | 0 | 5.2 | 7.9 | 9.1 | 9.1 | 7.3 | 7.0 |
| Yes | No | 0 | 2.6 | 3.4 | 2.8 | 4.7 | 1.8 | 2.5 |
| Yes | Yes | 0 | 0.0 | 0.0 | 0.0 | 0.7 | 0.0 | 0.0 |
| Yes | Yes | 1 | 0.0 | 0.0 | 0.0 | 2.3 | 0.0 | 1.2 |
| Yes | Yes | 3 | 0.0 | 0.0 | 1.4 | 1.9 | 1.9 | 1.6 |
| Yes | Yes | 10 | 0.0 | 1.2 | 1.2 | 0.0 | 0.0 | 1.9 |
| Yes | Yes | 30 | 1.2 | 0.0 | 0.0 | 2.1 | 1.6 | 1.6 |
| Yes | Yes | 100 | 1.2 | 2.1 | 0.0 | 1.9 | 2.8 | 1.4 |
| Yes | Yes | 300 | 1.4 | 3.3 | 1.4 | 2.8 | 2.1 | 3.0 |
| Yes | Yes | 1000 | 1.9 | N.D. | 1.6 | 2.3 | 2.6 | 4.2 |
| Yes | Yes | 1189 | N.D. | N.D. | N.D. | N.D. | 1.6 | 4.0 |
| Yes | Yes | 1633 | N.D. | N.D. | 2.1 | 3.5 | N.D. | N.D. |

*N.D. indicates not determined

### Example 20

### Evaluation of the Effect of 0322-0000-1069/1139 on the Cofactor Function of Protein S on FXa Inhibition by TFPI

Protein S has been reported to function as a cofactor for TFPI by augmenting TFPI inhibition of FXa activity (Hackeng T. M. et al. PNAS (2006) 103(9):3106-11). To investigate the effect of mAb 0322-0000-1069 and the corresponding Fab fragment 0322-0000-1139 on the cofactor activity of Protein S an FXa activity assay was established. In brief, human Protein S (50 nM, from Enzyme Research Laboratories) was incubated with lipid vesicles (25 μM; 25:75 POPS:POPC from Avanti Polar Lipids) and mAb/Fab (500 nM) 10 minutes at 25° C. Human full-length TFPI (5 nM) and S-2765 (200 μM, from Chromogenix) were added and the reactions were started with human FXa (0.5 nM; from Haematologic technologies). The experiment was conducted in 50 mM Hepes, pH 7.4; 0.1 M NaCl; 10 mM CaCl₂ supplemented with 1 mg/ml bovine serum albumin and 1 mg/ml PEG8000. Hydrolysis of the FXa specific substrate was followed over time by measuring the absorbance at 405 nm using a SpectraMax M2 instrument.

As reported previously Protein S augments the inhibitory activity of TFPI and neither 0322-0000-1069 nor the fab fragment, 0322-0000-1139 (data not shown) affects the cofactor function of Protein S towards TFPI. However, a LamG domain-targeting antibody 0322-0000-0032 completely abolishes the effect of Protein S (FIG. 12).

### Example 21

### Evaluation of the Interaction of Free Protein S and Protein S in Complex with 0322-0000-1069/0322-0000-1139 with TFPI

The binding of free human Protein S (from Enzyme Research Laboratories) and Protein S in complex with mAb 0322-0000-1069 or the corresponding Fab fragment 0322-0000-1139 to full-length human TFPI was investigated by surface plasmon resonance using a Biacore T200 instrument.

In brief, TFPI was immobilized on a CM5 biacore sensor chip by standard amine coupling chemistry. Binding of free Protein S (200 nM) or Protein S (200 nM) in complex with 0322-0000-1069 (200 nM) or 0322-0000-1139 (400 nM) was evaluated. As a control 0322-0000-0023 targeting the LamG domains of Protein S was included. The experiment was conducted in 10 mM Hepes, pH 7.4; 150 mM NaCl; 10 M CaCl₂ supplemented with 0.005% Tween20 and the chip was regenerated with glycine-HCl pH 2.5. The binding of Protein S to TFPI was not prevented by 0322-0000-1069 or 0322-0000-1139 whereas the binding of Protein S to TFPI was completely inhibited by 0322-0000-0023 (FIG. 13).

These observations are in good agreement with the known epitopes of the antibodies. 0322-0000-1069/0322-0000-1139 binds the EGF-1 domain of Protein S which is located far from the LamG domains which are known to mediate the binding to TFPI (Reglilska-Matveyev N. et al. Blood (2014) 123(25):3979-3987). 0322-0000-0023 targets the LamG domains and therefore most likely has overlapping epitope with TFPI.

### Example 22

### Humanization of Anti-Protein S mAb 0322-0000-0914

A 3D model of the Fab fragment representing the murine anti-protein S antibody 0322-0000-0914 (VH/VL also represented in chimeric mAb 0322-0000-1069) was build using standard techniques in MOE (available from www.chemcomp.com) and all residues within 4.5 Å of the effective CDR regions (VH: 31-35B, 50-59, 95-102; VL: 24-34, 50-56, 89-97) are defined as Mask residues (numbering according to Kabat). Mask residues are all potentially important for sustaining the binding in the CDRs. The effective CDR regions are defined based on the majority of interaction patterns observed in antigen-antibody 3D structures available in the public domain.

The mask residues for 0322-0000-0914 include positions:
1-4, 23-37, 47, 50-59, 69-71, 73, 76, 78, 91-103 for the heavy chain and
1-5, 22-36, 46-60, 62, 63, 65, 69-71, 87-98 for the light chain (numbering according to Kabat).

Using germline homology searches (germline sequences can be found at http://www.imgtorg) and manual evaluation of sequence eligibility IGHV1-46*03 and J6*02 were identified as an appropriate human germline combination for the heavy chain and IGKV3-11*01 and JK4*02 were identified as the appropriate human germline combination for the light chain, although other germlines might also be eligible as humanization scaffolds.
The humanisation was then performed according to the following scheme and is outlined in FIG. 14 (light chain) and FIG. 15 (heavy chain):
Residues outside the mask are taken as human.
Residues inside the mask and inside the Kabat CDR are taken as murine.
Residues inside the mask and outside the Kabat CDR with mouse/human germline consensus are taken as the consensus sequence.
Residues inside the mask and outside the Kabat CDR with mouse/human germline difference are taken either as:
   human, i.e. not subjected to back mutation, or
   murine, i.e subject to potential back mutations

The humanized light and heavy chain variable regions derived from the described humanization scheme are listed below with and without potential back mutations. The CDR regions and the individual back mutations are also listed.

Following the humanization scheme described above, the initial humanized 0322-0000-0914 VH construct carries a VH CDR2 designed according to a minimal CDR grafting strategy, in which VH CDR2 is grafted in a shorter “effective CDR” version (residue 50-59) than the Kabat definition (residue 50-65). This results in the introduction of human germline sequence in the distal part of the heavy chain CDR2 corresponding to residues 60-65 (numbering according to Kabat). An alternative version of the humanized 0322-0000-0914 VH construct, designed according to the humanization scheme, but carrying a full murine CDR2 in the heavy chain was also generated (VH CDR2*) (see CDR sequences below).

### Humanized VL Regions

HZ 0914_VL
(SEQ ID NO: 49)
EIVLTQSPATLSLSPGERATLSCRASSSVSYMYVVYQQKPGQAPRLL
IYATSNLASGIPARFSGSGSGTDFTLTISSLEPEDFAVYYCQQWSSI
PPTFGGGTKVEIK

### CDR Region of the Humanized VL Region

CDR1:
(residues 24-33
SEQ ID NO: 49)
RASSSVSYMY
CDR2:
(residues 49-55 of
SEQ ID NO: 49)
ATSNLAS
CDR3:
(residues 88-96 of
SEQ ID NO: 49)
QQWSSIPPT

List of potential back mutations in the humanized VL region (numbering according to Kabat) as highlighted in grey in the sequence above.

### HZ 0914_VL E1Q

### HZ 0914_VL T5S

### HZ 0914_VL S22T

### HZ 0914_VL L46P

### HZ 0914_VL L47W

### HZ 0914_VL I58V

### HZ 0914_VL D70S

### HZ 0914_VL F71Y

### Humanized VH Regions

HZ 0914_VH
(SEQ ID NO: 50)
QVQLVQSGAEVKKPGASVKVSCKASGYTFTSYWINWVRQAPGQGLEW
MGRIDPYDSETHYAQKFQGRVTMTRDTSTSTVYMELSSLRSEDTAVY
YCARWGGSGYAMDYWGQGTTVTVSS

### CDR Region of the Humanized VH Region

CDR1:
(residues 31-35 of
SEQ ID NO: 50)
SYWIN
CDR2:
(residues 50-66 of
SEQ ID NO: 50)
RIDPYDSETHYAQKFQG
CDR3:
(residues 99-108 of
SEQ ID NO: 50)
WGGSGYAMDY
CDR2*:
(residues 50-66 of
SEQ ID NO: 41)
RIDPYDSETHYNQKFKD

List of potential back mutations in the humanized VH region (numbering according to Kabat) as highlighted in grey on sequence above.

### HZ 0914_VH A60N

### HZ 0914_VH M69L

### HZ 0914_VH R71V

### HZ 0914_VH T73K

### HZ 0914_VH V78A

### Example 23

### Generation of Expression Vectors for Transient Expression of Humanized Anti-Protein S Antibody Variants

DNA fragments representing the coding regions of the humanized 0322-0000-0914 VH and VL regions were synthesized (GENEART AG/Life Technologies) according to the humanization scheme described above.

The sequences for the humanized VL region were obtained with/without the 8 potential back mutations. A set of 8 variants carrying the individual 8 back mutations in the VL region was also synthesised. A large number of variants carrying combinations of 2, 3, 4, 5, 6, and 7 of the identified potential VL back mutations were generated by site-directed mutagenesis using mutagenic primers and the QuickChange® Lightning Site-Directed or QuickChange® Lightning Multi Site-Directed Mutagenesis kits from Agilent. The kits were used according to the manufacturer's protocol. The combinatorial mutations were either generated by using mutagenic primers design to add back mutations to variants carrying single back mutations or mutagenic primers designed to remove back mutations from the variant carrying all 8 back mutations.

The sequences for the humanized VH region were obtained with/without the 5 potential back mutations. A series of 5 variants carrying the individual 5 back mutations in the VH region and the combinatorial library of 25 variants carrying combinations of 2, 3 and 4 of the identified potential VH back mutations were also synthesised. In addition a humanized VH construct carrying the full murine VH CDR2 (listed as CDR2* above) but no additional back mutations was also synthesised.

For both VL and VH constructs the leader peptide sequence of human CD33 was include in lieu of the natural immunoglobulin signal peptide sequences and a Kozak sequence (5′-GCCGCCACC-3′) was introduced immediately upstream of the ATG start codon.

pTT-based expression vectors were generated for transient expression of the humanized anti-Protein S antibody as a human kappa/IgG4(S241P) isotype. The proline mutation at position 241 (numbering according to Kabat, corresponding to residue 228 per the EU numbering system (Edelman G. M. et al., Proc. Natl. Acad. USA 63, 78-85 (1969)) was included in the IgG4 hinge region to eliminate formation of monomeric antibody fragments, i.e. “half-antibodies” comprising one LC and one HC.

The VH fragments were excised from the GENEART cloning vectors using standard restriction based cloning (HindIII/NheI restriction enzyme digest) and cloned in-frame into a linearized pTT-based vector containing the sequence for a human IgG4(S241P) CH domain (HindIII/NheI restriction enzyme digest). The VL fragments were excised from the GENEART cloning vectors using standard restriction based cloning (HindIII/BsiWI restriction enzyme digest) and cloned in-frame into a linearized pTT-based vector containing the sequence for a human kappa CL domain (HindIII/BsiWI restriction enzyme digest). Assembled vectors were subsequently transformed into E. coli for selection. The sequences of the final constructs were verified by DNA sequencing. As mentioned above variants carrying combinations of VL back mutations were generated by site-directed mutagenesis using mutagenic primers and the QuickChange® Lightning Site-Directed or QuickChange® Lightning Multi Site-Directed Mutagenesis kits from Agilent.

Humanization variants were expressed transiently in EXPI293F cells (Life Technologies) by co-transfection of the different pTT-based LC/HC expression vectors as described in Example 11.

The humanization process is carried out as an iterative protein engineering process. The iterative steps of variant generation, production and testing are outlined below:

Step 1: CDR grafted humanization variant (0322-0000-1152) compared with the murine-human chimeric version (0322-0000-1069) of the original murine antibody (0322-0000-0914). The murine-human chimeric antibody is used as reference throughout the humanization process. The CDR grafted variant carrying all 5 potential VH and all 8 potential VL back mutations (0322-0000-1155) is also tested along with variants carrying either all 5 VH back mutations, but no back mutations in the CDR grafted light chain (0322-0000-1154) or all 8 VL back mutations, but no back mutations in the CDR grafted heavy chain (0322-0000-1153).

Step 2: Variants carrying the individual 8 potential VL or 5 potential VH back mutations (e.g. 0322-0000-1166) are compared with the murine-human chimeric mAb (0322-0000-1069) and the humanization variants carrying a full set of 5 VH or 8 VL back mutations (0322-0000-1154 or 0322-0000-1153, respectively).

Step 3: In multiple iterations, humanization variants with combinations of different VL back mutations (e.g. 0322-0000-1223) or VH back mutations as wells as humanization variants with combination of different VL and VH mutations (e.g. 0322-0000-1201) were tested and compared to previously identified variants and the murine-human chimeric mAb (0322-0000-1069) reference. Table 12 shows preferred humanization variants.

The humanization variants were tested in binding, functional assays and evaluated for biophysical/chemical properties and immunogenicity.

In order to avoid potential isoAsp sites in the sequence of the antibody of the invention D55 of SEQ ID NO: 50 (i.e. the humanized heavy chain) this residue may in one embodiment be substituted with a different amino acid residue which is not cysteine (C).

TABLE 12
Selected humanization variants
Back mutations are numbered according to Kabat or according to corresponding SEQ ID NO.

| | | | LC back | | | HC back | |
|---|---|---|---|---|---|---|---|
| | | | mutation | LC back | | mutation | HC back |
| | | | according | mutation | | according | mutation |
| mAb | mAb | LC SEQ | SEQ ID | according | HC SEQ | SEQ ID | according |
| ID | description | ID NO: | NO | to Kabat | ID NO: | NO | to Kabat |
| 0322- | HZ 0914_ LC | 56 | | | 57 | | |
| 0000- | hKappa/HZ | | | | | | |
| 1152 | 0914_HC | | | | | | |
| | hIgG4(5241P) | | | | | | |
| 0322- | HZ 0914_ LC | 58 | L45P | L46P | 57 | | |
| 0000- | L46P hKappa/HZ | | | | | | |
| 1166 | 0914_HC | | | | | | |
| | hIgG4(5241P) | | | | | | |
| 0322- | HZ 0914_ LC | 58 | L45P | L46P | 59 | M70L, | M69L, |
| 0000- | L46P hKappa/HZ | | | | | V79A | V78A |
| 1201 | 0914_HC | | | | | | |
| | M69L, V78A | | | | | | |
| | hIgG4(5241P) | | | | | | |
| 0322- | HZ 0914_ LC | 60 | L45P, | L46P, | 57 | | |
| 0000- | L46P, L47W | | L46W | L47W | | | |
| 1223 | hKappa/HZ | | | | | | |
| | 0914_HC | | | | | | |
| | hIgG4(S241P) | | | | | | |
| 0322- | HZ 0914_ LC | 58 | L45P | L46P | 61 | M70L, | M69L, |
| 0000- | L46P hKappa/HZ | | | | | R72V, | R71V, |
| 1238 | 0914_HC | | | | | V79A | V78A |
| | M69L, R71V, | | | | | | |
| | V78A | | | | | | |
| | hIgG4(S241P) | | | | | | |
| 0322- | HZ 0914_ LC | 58 | L45P | L46P | 62 | M70L, | M69L, |
| 0000- | L46P hKappa/HZ | | | | | T74K, | T73K, |
| 1239 | 0914_HC | | | | | V79A | V78A |
| | M69L, T73K, | | | | | | |
| | V78A | | | | | | |
| | hIgG4(S241P) | | | | | | |
| 0322- | HZ 0914_ LC | 60 | L45P, | L46P, | 59 | M70L, | M69L, |
| 0000- | L46P, L47W | | L46W | L47W | | V79A | V78A |
| 1246 | hKappa/HZ | | | | | | |
| | 0914_HC | | | | | | |
| | M69L, V78A | | | | | | |
| | hIgG4(S241P) | | | | | | |
| 0322- | HZ 0914_ LC | 60 | L45P, | L46P, | 61 | M70L, | M69L, |
| 0000- | L46P, L47W | | L46W | L47W | | R72V, | R71V, |
| 1248 | hKappa/HZ | | | | | V79A | V78A |
| | 0914_HC | | | | | | |
| | M69L, R71V, | | | | | | |
| | V78A | | | | | | |
| | hIgG4(S241P) | | | | | | |
| 0322- | HZ 0914_ LC | 60 | L45P, | L46P, | 62 | M70L, | M69L, |
| 0000- | L46P, L47W | | L46W | L47W | | T74K, | T73K, |
| 1249 | hKappa/HZ | | | | | V79A | V78A |
| | 0914_HC | | | | | | |
| | M69L, T73K, | | | | | | |
| | V78A | | | | | | |
| | hIgG4(S241P) | | | | | | |

### Example 24

### Evaluation of Efficacy of Anti-Protein S Humanization Variants in Haemophilic Plasma Using the Thrombin Generation Assay

The humanization variants of 0322-0000-1069 were evaluated for their ability to improve thrombin generation in haemophilic plasma compared to 0322-0000-1069 using the Calibrated Automated Thrombogram® (CAT) system.

In brief, humanization variants were mixed with human haemophilia A (HA) plasma (George King Bio-medical Inc) spiked with activated protein C (APC; Haematologic Technologies, Inc). The concentrations of antibodies in the plasma varied between 3 and 1000 nM, and the APC concentration in the plasma was 5 nM. Next, 80 μl of this plasma mixture was incubated with 20 μl PPP-reagent (Thrombinoscope) containing tissue factor and phospholipids at a final concentration of 5 pM and 4 μM, respectively, for 10 min at 37° C. in duplicate, in Immulon 2 HB-High Binding 96-well U-bottom plates (VWR). In control wells, 80 μl plasma (without antibody or APC) was mixed with 20 μl thrombin calibrator. The reaction was initiated with the addition of 20 μl pre-warmed (37° C.) FluCa reagent (Thrombinoscope) containing CaCl₂ and a fluorescent thrombin substrate. Fluorescence was monitored every 20 seconds for 60 min, and analysis was performed using Thrombinoscope Analysis Version 5.0. The software provides a thrombogram calculated from the first derivative of the integral fluorescence curve, as well as parameters associated with the thrombogram such as peak thrombin, measured in nM.

A humanized variant containing no back mutations (BM) (0322-0000-1152) was compared to the murine-human IgG4 chimer 0322-0000-1069 (cf. table 13).

TABLE 13
Thrombin generation results from the first round of humanization

| | Anti- | Peak thrombin* (nM) | | |
|---|---|---|---|---|
| | body | Plasma | | |
| Plasma | (nM) | controls | 0322-0000-1069 | 0322-0000-1152 |
| normal | 0 | 185.7 (28.9) | | |
| HA | 0 | 76.7 (20.7) | | |
| HA + APC | 0 | 45.9 (4.6) | | |
| HA + APC | 1000 | | 185.5 (1.0) | 129.2 (20.4) |
| HA + APC | 300 | | 184.4 (4.3) | 89.8 (18.3) |
| HA + APC | 100 | | 167.2 (8.6) | 60.2 (14.5) |
| HA + APC | 30 | | 65.1 (8.0) | 41.0 (7.7) |
| HA + APC | 10 | | 46.3 (3.3) | 32.0 (5.4) |
| HA + APC | 3 | | 39.2 (2.6) | 32.2 (8.1) |

*data are mean (standard deviation) with n = 3

Initially, a variant, namely 0322-0000-1166, was able to improve thrombin generation activity over a variant without any BM, which contained a L46P mutation in the LC.

As part of the further humanization, combinations of back mutations (BM) were introduced, all containing the L46P mutation in the LC. All combinations that were tested had the ability to improve thrombin generation, and most combinations generated levels of thrombin that were equivalent or superior to 0322-0000-1166.

Eight final combinations were compared directly using thrombin generation (cf. table 14). All 8 of these molecules had activity that was comparable to 0322-0000-1069.

TABLE 14
Thrombin generation results from the final set of humanization variants in HA plasma + 5 nM APC

| | 0322-0000- | Peak thrombin* (nM) | | | | | | | |
|---|---|---|---|---|---|---|---|---|---|
| | 1069 | 0322- | 0322- | 0322- | 0322- | 0322- | 0322- | 0322- | 0322- |
| Antibody | and Plasma | 0000- | 0000- | 0000- | 0000- | 0000- | 0000- | 0000- | 0000- |
| (nM) | controls | 1166 | 1223 | 1201 | 1246 | 1238 | 1249 | 1239 | 1248 |
| 1000 | 214.4 | 202.3 | 212.2 | 217.0 | 221.2 | 207.9 | 212.8 | 201.9 | N.D. |
| | (1.9) | (21.2) | (8.4) | (8.2) | (13.7) | (11.0) | (18.6) | (32.6) | |
| 625 | | N.D. | N.D. | N.D. | N.D. | N.D. | N.D. | N.D. | 243.5 |
| | | | | | | | | | (3.0) |
| 300 | 223.9 | 191.5 | 200.5 | 220.0 | 214.4 | 213.4 | 207.5 | 200.4 | 215.9 |
| | (17.6) | (24.8) | (15.8) | (15.3) | (26.5) | (24.4) | (29.1) | (31.3) | (27.3) |
| 100 | 155.2 | 150.4 | 125.5 | 166.0 | 139.9 | 161.3 | 135.0 | 186.2 | 135.7 |
| | (13.1) | (23.8) | (18.4) | (8.1) | (20.5) | (25.3) | (28.9) | (28.8) | (31.0) |
| 30 | 72.4 | 75.6 | 68.7 | 72.2 | 72.8 | 82.9 | 74.0 | 143.7 | 61.6 |
| | (21.3) | (19.2) | (13.3) | (3.9) | (24.0) | (18.7) | (14.3) | (29.6) | (5.4) |
| 10 | N.D. | N.D. | 47.7 | 69.2 | 39.8 | 39.9 | 40.5 | 67.2 | 50.3 |
| | | | (19.9) | (40.4) | (18.0) | (19.7) | (20.1) | (32.6) | (35.1) |
| 0 in HA | 82.0 | | | | | | | | |
| | (39.1) | | | | | | | | |
| 0 in HA + | 40.1 | | | | | | | | |
| APC | (16.1) | | | | | | | | |
| 0 in NHP | 156.4 | | | | | | | | |
| | (36.8) | | | | | | | | |

*data are mean (standard deviation); n = 3
N.D. = not determined

### Example 25

### Corresponding ID Numbers and Names

The ID numbers and names of recombinantly expressed and hybridoma derived antibodies are listed in table 15. The full ID numbers (e.g. 0322-0000-0914) as well as abbreviated IDs containing the last digits of the ID (e.g. mAb 0914 or mAb 914) are used throughout the document.

TABLE 15
Overview of corresponding ID numbers and names

| | Recombinantly | | | SEQ ID NO of |
|---|---|---|---|---|
| Recombinantly | expressed | | | light- and |
| expressed | murine-human | | | heavy chain |
| mouse IgG1 | chimeric | Hybridoma | Hybridoma | variable domains |
| antibodies ID | antibodies | ID | name | (VL, VH) |
| 0322-0000-0916 | 0322-0000-0114 | 0322-0000-0010 | 2F4A1 | 10, 11 |
| 0322-0000-0919 | | 0322-0000-0208 | 6F230A10 | 42, 43 |
| 0322-0000-0921 | | 0322-0000-0177 | 6F159A11 | 34, 35 |
| 0322-0000-0914 | 0322-0000-1069* | 0322-0000-0203 | 6F216A3 | 40, 41 |
| 0322-0000-0912 | 0322-0000-1068 | 0322-0000-0158 | 6F138A1/A3 | 26, 27 |
| 0322-0000-0909 | | 0322-0000-0092 | 3F2A1 | 14, 15 |
| 0322-0000-0918 | 0322-0000-0115 | 0322-0000-0017 | 2F82A1 | 12, 13 |
| 0322-0000-0920 | | 0322-0000-0123 | 6F101A1/A3 | 20, 21 |
| 0322-0000-0915 | | 0322-0000-0224 | 6F265A1 | 44, 45 |
| 0322-0000-0910 | 0322-0000-1067 | 0322-0000-0184 | 6F170A2 | 36, 37 |
| 0322-0000-0911 | | 0322-0000-0173 | 6F153A2 | 32, 33 |
| 0322-0000-0913 | | 0322-0000-0147 | 6F128A2 | 24, 25 |

*The recombinantly expressed murine-human chimeric Fab fragment of 0322-0000-1069 is identified as 0322-0000-1139. The identification of mAbs and Fab fragments may in general be abbreviated to mAb 1069 or Fab 1139 by using the last four numbers in the ID listed in the above table.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. An inhibitor capable of specifically binding in the EGF1-3 region of human Protein S for use in the treatment of coagulopathy in a human subject.

2. The inhibitor for use according to claim 1 wherein said inhibitor is capable of specifically binding in the EGF 1 region of human Protein S for use in the treatment of coagulopathy in a human subject.

3. The inhibitor for use according to claim 1 wherein the inhibitor is an antibody or antigen-binding fragment thereof

4. An antibody or antigen-binding fragment thereof capable of specifically binding in the EGF1 region of human Protein S wherein said binding region comprises one or more amino acid residues selected from the group consisting of W36, E39, K40, C41, E42 and F43 of SEQ ID NO: 2.

5. The antibody or antigen-binding fragment thereof according to claim 4 wherein said antibody or antigen-binding fragment thereof is capable of specifically binding amino acid residues W36, E39, K40, and one or more of amino acid residues C41, E42 and F43 of SEQ ID NO: 2.

6. An antibody or antigen-binding fragment thereof which is capable of specifically binding in the EGF1 region of human Protein S wherein the light chain of said antibody or antigen-binding fragment comprises a CDR3 sequence comprising amino acid residues 88-96 of SEQ ID NO: 49 (QQWSSIPPT), wherein one or two of said residues can be substituted with a different residue, and the heavy chain of said antibody or antigen-binding fragment comprises a CDR3 sequence comprising amino acid residues 99-108 of SEQ ID NO: 50 (WGGSGYAMDY), wherein one or two of said residues can be substituted with a different residue.

7. An antibody or antigen-binding fragment thereof according to claim 6, wherein the light chain of said antibody or antigen-binding fragment comprises:
a CDR1 sequence comprising amino acid residues 24-33 SEQ ID NO: 49 (RASSSVSYMY), wherein one or two of said residues can be substituted with a different residue, and/or
a CDR2 sequence comprising amino acid residues 49-55 of SEQ ID NO: 49 (ATSNLAS), wherein one or two of said residues can be substituted with a different residue, and/or
a CDR3 sequence comprising amino acid residues 88-96 of SEQ ID NO: 49 (QQWSSIPPT), wherein one or two of said residues can be substituted with a different residue; and the heavy chain of said antibody or antigen-binding fragment comprises:
a CDR1 sequence comprising amino acid residues 31-35 of SEQ ID NO: 50 (SYWIN), wherein one or two of said residues can be substituted with a different residue, and/or
a CDR2 sequence comprising amino acid residues 50-66 of SEQ ID NO: 50 (RIDPYDSETHYAQKFQG), wherein one or two of said residues can be substituted with a different residue, and/or
a CDR3 sequence comprising amino acid residues 99-108 of SEQ ID NO: 50 (WGGSGYAMDY) wherein one or two of said residues can be substituted with a different residue.

8. An antibody or antigen-binding fragment thereof according to claim 6, wherein the light chain variable domain (VL) of said antibody or antigen-binding fragment comprises SEQ ID NO: 49, wherein amino acid residue L45 is substituted with P, and optionally L46 is substituted with W; and the heavy chain variable domain (VH) of said antibody or antigen-binding fragment comprises SEQ ID NO: 50, optionally further comprising one or more of the substitutions selected from a group consisting of M70L, R72V, T74K and V79A.

9. The antibody or antigen-binding fragment thereof according to claim 6, wherein the light chain variable domain (VL) of said antibody comprises SEQ ID NO: 51 or 53, and the heavy chain heavy chain variable domain (VH) of said antibody comprises SEQ ID NO: 50, 52, 54 or 55.

10. The antibody or antigen-binding fragment thereof according to claim 9 wherein the light chain variable domain (VL) of said antibody comprises SEQ ID NO: 51 and the heavy chain variable domain (VH) of said antibody comprises SEQ ID NO: 50.

11. The antibody or antigen-binding fragment thereof according to claim 9 wherein the light chain variable domain (VL) of said antibody comprises SEQ ID NO: 51 and the heavy chain variable domain (VH) of said antibody comprises SEQ ID NO: 52.

12. The antibody or antigen-binding fragment thereof according to claim 9 wherein the light chain variable domain (VL) of said antibody comprises SEQ ID NO: 51, and the heavy chain variable domain (VH) of said antibody comprises SEQ ID NO: 54.

13. The antibody or antigen-binding fragment thereof according to claim 9 wherein the light chain variable domain (VL) of said antibody comprises SEQ ID NO: 51 and the heavy chain variable domain (VH) of said antibody comprises SEQ ID NO: 55.

14. The antibody or antigen-binding fragment thereof according to claim 9 wherein the light chain variable domain (VL) of said antibody comprises SEQ ID NO: 53 and the heavy chain variable domain (VH) of said antibody comprises SEQ ID NO: 50.

15. The antibody or antigen-binding fragment thereof according to claim 9 wherein the light chain variable domain (VL) of said antibody comprises SEQ ID NO: 53 and the heavy chain variable domain (VH) of said antibody comprises SEQ ID NO: 52.

16. The antibody or antigen-binding fragment thereof according to claim 9 wherein the light chain variable domain (VL) of said antibody comprises SEQ ID NO: 53 and the heavy chain variable domain (VH) of said antibody comprises SEQ ID NO: 54.

17. The antibody or antigen-binding fragment thereof according to claim 9 wherein the light chain variable domain (VL) of said antibody comprises SEQ ID NO: 53 and the heavy chain variable domain (VH) of said antibody comprises SEQ ID NO: 55.

18. The antibody or antigen-binding fragment thereof according to claim 7 wherein the heavy chain variable domain (VH) CDR2 amino acid residue D55 of SEQ ID NO: 50 optionally may be substituted with a different amino acid residue which is not C.

19. The antibody according to claim 3 wherein the antibody is a monoclonal antibody.

20. A polynucleotide which encodes the inhibitor according to claim 1.

21. A pharmaceutical composition comprising the inhibitor according to claim 1 and a pharmaceutically acceptable carrier or diluent.

22. The antibody or antigen-binding fragment thereof according to claim 4 for use in the treatment of coagulopathy in a human subject.

23. The antibody or antigen-binding fragment thereof according to claim 22 for use in the treatment of haemophilia in a human subject.

24. A eukaryotic cell which expresses the inhibitor according to claim 1.

25. An antibody, or an antigen-binding fragment thereof, which competes with a reference antibody in binding to human Protein S, wherein the reference antibody comprises a heavy chain variable region and a light chain variable region according to claim 8.

26. A polynucleotide which encodes the antibody or antigen-binding fragment thereof according to claim 4.

27. A pharmaceutical composition comprising the antibody or antigen-binding fragment thereof according to claim 4 and a pharmaceutically acceptable carrier or diluent.

28. A eukaryotic cell which expresses the antibody or antigen-binding fragment thereof according to claim 4.
